SEQUENCE LISTING (504 sequences)

SEQ ID NO: 1 - RNA, 22 nt, homo sapiens
ugagguaguagguuguauaguu

SEQ ID NO: 2 - RNA, 22 nt, homo sapiens
ugagguaguagguugugugguu

SEQ ID NO: 3 - RNA, 22 nt, homo sapiens
ugagguaguagguuguaugguu

SEQ ID NO: 4 - RNA, 22 nt, homo sapiens
agagguaguagguugcauaguu

SEQ ID NO: 5 - RNA, 22 nt, homo sapiens
ugagguaggagguuguauaguu

SEQ ID NO: 6 - RNA, 22 nt, homo sapiens
ugagguaguagauuguauaguu

SEQ ID NO: 7 - RNA, 22 nt, homo sapiens
ugagguaguaguuuguacaguu

SEQ ID NO: 8 - RNA, 22 nt, homo sapiens
ugagguaguaguuugugcuguu

SEQ ID NO: 9 - RNA, 22 nt, homo sapiens
uggaauguaaagaaguauguau

SEQ ID NO: 10 - RNA, 22 nt, homo sapiens
aacccguagauccgaacuugug

SEQ ID NO: 11 - RNA, 22 nt, homo sapiens
uacaguacugugauaacugaag

SEQ ID NO: 12 - RNA, 23 nt, homo sapiens
agcagcauuguacagggcuauga

SEQ ID NO: 13 - RNA, 23 nt, homo sapiens
ucaaaugcucagacuccuguggu

SEQ ID NO: 14 - RNA, 23 nt, homo sapiens
aaaagugcuuacagugcagguag

SEQ ID NO: 15 - RNA, 21 nt, homo sapiens
uaaagugcugacagugcagau

SEQ ID NO: 16 - RNA, 21 nt, homo sapiens
agcagcauuguacagggcuau

SEQ ID NO: 17 - RNA, 22 nt, homo sapiens
uacccuguagauccgaauuugu

SEQ ID NO: 18 - RNA, 22 nt, homo sapiens
uacccuguagaaccgaauuugu

SEQ ID NO: 19 - RNA, 22 nt, homo sapiens
aagcauucuuucauugguuggu

SEQ ID NO: 20 - RNA, 22 nt, homo sapiens
uuuccggcucgcgugggugugu

SEQ ID NO: 21 - RNA, 22 nt, homo sapiens
agaggauacccuuuguauguuc

SEQ ID NO: 22 - RNA, 23 nt, homo sapiens
gggaugguagaccggugacgugc

SEQ ID NO: 23 - RNA, 21 nt, homo sapiens
uaggacacauggucuacuucu

SEQ ID NO: 24 - RNA, 23 nt, homo sapiens
uggagugugacaaugguguuugu

SEQ ID NO: 25 - RNA, 21 nt, homo sapiens
uaaggcacgcggugaaugcca

SEQ ID NO: 26 - RNA, 21 nt, homo sapiens
aagugaucuaaaggccuacau

SEQ ID NO: 27 - RNA, 22 nt, homo sapiens
acccgucccguucguccccgga

SEQ ID NO: 28 - RNA, 22 nt, homo sapiens
acgcccuucccccccuucuuca

SEQ ID NO: 29 - RNA, 21 nt, homo sapiens
acggugcuggauguggccuuu

SEQ ID NO: 30 - RNA, 21 nt, homo sapiens
acucuagcugccaaaggcgcu

SEQ ID NO: 31 - RNA, 21 nt, homo sapiens
agaaggaaauugaauucauuu

SEQ ID NO: 32 - RNA, 23 nt, homo sapiens
aggaugagcaaagaaaguagauu

SEQ ID NO: 33 - RNA, 22 nt, homo sapiens
cggaugagcaaagaaagugguu

SEQ ID NO: 34 - RNA, 22 nt, homo sapiens
cuaaagagaagucaaugcauga

SEQ ID NO: 35 - RNA, 21 nt, homo sapiens
aguuaggauuaggucguggaa

SEQ ID NO: 36 - RNA, 22 nt, homo sapiens
ucccugagacccuuuaaccugu

SEQ ID NO: 37 - RNA, 22 nt, homo sapiens
ucccugagacccuaacuuguga

SEQ ID NO: 38 - RNA, 22 nt, homo sapiens
ucguaccgugaguaauaaugcg

SEQ ID NO: 39 - RNA, 22 nt, homo sapiens
augguacccuggcauacugagu

SEQ ID NO: 40 - RNA, 22 nt, homo sapiens
caagucuuauuugagcaccugu

SEQ ID NO: 41 - RNA, 23 nt, homo sapiens
ccucagggcuguagaacagggcu

SEQ ID NO: 42 - RNA, 22 nt, homo sapiens
cuggacugagccgugcuacugg

SEQ ID NO: 43 - RNA, 22 nt, homo sapiens
ucggauccgucugagcuuggcu

SEQ ID NO: 44 - RNA, 23 nt, homo sapiens
cuggagauauggaagagcugugu

SEQ ID NO: 45 - RNA, 22 nt, homo sapiens
cuuggcaccuagcaagcacuca

SEQ ID NO: 46 - RNA, 22 nt, homo sapiens
uacguagauauauauguauuuu

SEQ ID NO: 47 - RNA, 22 nt, homo sapiens
uaguacugugcauaucaucuau

SEQ ID NO: 48 - RNA, 21 nt, homo sapiens
ucacagugaaccggucucuuu

SEQ ID NO: 49 - RNA, 22 nt, homo sapiens
ucuacaaaggaaagcgcuuucu

SEQ ID NO: 50 - RNA, 22 nt, homo sapiens
gaaagcccauguuuguauugga

SEQ ID NO: 51 - RNA, 21 nt, homo sapiens
ugcaggaccaagaugagcccu

SEQ ID NO: 52 - RNA, 21 nt, homo sapiens
ugcuggaucagugguucgagu

SEQ ID NO: 53 - RNA, 22 nt, homo sapiens
uggaguccaggaaucugcauuu

SEQ ID NO: 54 - RNA, 22 nt, homo sapiens
aagcccuuaccccaaaaaguau

SEQ ID NO: 55 - RNA, 22 nt, homo sapiens
aagcccuuaccccaaaaagcau

SEQ ID NO: 56 - RNA, 22 nt, homo sapiens
ucuggguggucuggagauuugu

SEQ ID NO: 57 - RNA, 22 nt, homo sapiens
ugugagguuggcauuguugucu

SEQ ID NO: 58 - RNA, 21 nt, homo sapiens
uuaggccgcagaucuggguga

SEQ ID NO: 59 - RNA, 22 nt, homo sapiens
uuagggcccuggcuccaucucc

SEQ ID NO: 60 - RNA, 20 nt, homo sapiens
uucauucggcuguccagaug

SEQ ID NO: 61 - RNA, 24 nt, homo sapiens
uugcagcugccugggagugacuuc

SEQ ID NO: 62 - RNA, 23 nt, homo sapiens
uuuuagagacggggucuugcucu

SEQ ID NO: 63 - RNA, 22 nt, homo sapiens
cgguuugaggcuacagugagau

SEQ ID NO: 64 - RNA, 22 nt, homo sapiens
uuuucaacucuaaugggagaga

SEQ ID NO: 65 - RNA, 22 nt, homo sapiens
ccaccuccccugcaaacgucca

SEQ ID NO: 66 - RNA, 21 nt, homo sapiens
ucgaccggaccucgaccggcu

SEQ ID NO: 67 - RNA, 22 nt, homo sapiens
cagugcaauguuaaaagggcau

SEQ ID NO: 68 - RNA, 22 nt, homo sapiens
cagugcaaugaugaaagggcau

SEQ ID NO: 69 - RNA, 22 nt, homo sapiens
uaacagucuacagccauggucg

SEQ ID NO: 70 - RNA, 22 nt, homo sapiens
ucaaaacugaggggcauuuucu

SEQ ID NO: 71 - RNA, 23 nt, homo sapiens
uuugguccccuucaaccagcugu

SEQ ID NO: 72 - RNA, 21 nt, homo sapiens
uuugguccccuucaaccagcu

SEQ ID NO: 73 - RNA, 22 nt, homo sapiens
ugugacugguugaccagagggg

SEQ ID NO: 74 - RNA, 23 nt, homo sapiens
uauggcuuuuuauuccuauguga

SEQ ID NO: 75 - RNA, 23 nt, homo sapiens
uauggcuuuucauuccuauguga

SEQ ID NO: 76 - RNA, 23 nt, homo sapiens
acuccauuuguuuugaugaugga

SEQ ID NO: 77 - RNA, 23 nt, homo sapiens
uuauugcuuaagaauacgcguag

SEQ ID NO: 78 - RNA, 23 nt, homo sapiens
agcugguguugugaaucaggccg

SEQ ID NO: 79 - RNA, 23 nt, homo sapiens
ucuacagugcacgugucuccagu

SEQ ID NO: 80 - RNA, 22 nt, homo sapiens
accacaggguagaaccacggac

SEQ ID NO: 81 - RNA, 23 nt, homo sapiens
uaacacugucugguaaagauggc

SEQ ID NO: 82 - RNA, 23 nt, homo sapiens
uguaguguuuccuacuuuaugga

SEQ ID NO: 83 - RNA, 21 nt, homo sapiens
ugagaugaagcacuguagcuc

SEQ ID NO: 84 - RNA, 20 nt, homo sapiens
uacaguauagaugauguacu

SEQ ID NO: 85 - RNA, 23 nt, homo sapiens
guccaguuuucccaggaaucccu

SEQ ID NO: 86 - RNA, 22 nt, homo sapiens
cuccguuugccuguuucgcuga

SEQ ID NO: 87 - RNA, 22 nt, homo sapiens
ugagaacugaauuccauggguu

SEQ ID NO: 88 - RNA, 22 nt, homo sapiens
ugagaacugaauuccauaggcu

SEQ ID NO: 89 - RNA, 21 nt, homo sapiens
gugugcggaaaugcuucugcu

SEQ ID NO: 90 - RNA, 22 nt, homo sapiens
ucagugcacuacagaacuuugu

SEQ ID NO: 91 - RNA, 22 nt, homo sapiens
ucagugcaucacagaacuuugu

SEQ ID NO: 92 - RNA, 23 nt, homo sapiens
ucuggcuccgugucuucacuccc

SEQ ID NO: 93 - RNA, 22 nt, homo sapiens
ucucccaacccuuguaccagug

SEQ ID NO: 94 - RNA, 21 nt, homo sapiens
ucgaggagcucacagucuagu

SEQ ID NO: 95 - RNA, 21 nt, homo sapiens
ucagugcaugacagaacuugg

SEQ ID NO: 96 - RNA, 22 nt, homo sapiens
uugcauagucacaaaagugauc

SEQ ID NO: 97 - RNA, 22 nt, homo sapiens
aaaaccgucuaguuacaguugu

SEQ ID NO: 98 - RNA, 22 nt, homo sapiens
aaucauacacgguugaccuauu

SEQ ID NO: 99 - RNA, 23 nt, homo sapiens
uuaaugcuaaucgugauaggggu

SEQ ID NO: 100 - RNA, 21 nt, homo sapiens
uagcagcacauaaugguuugu

SEQ ID NO: 101 - RNA, 22 nt, homo sapiens
uagcagcacaucaugguuuaca

SEQ ID NO: 102 - RNA, 22 nt, homo sapiens
uagcagcacguaaauauuggcg

SEQ ID NO: 103 - RNA, 23 nt, homo sapiens
caaagugcuuacagugcagguag

SEQ ID NO: 104 - RNA, 23 nt, homo sapiens
aacauucaacgcugucggugagu

SEQ ID NO: 105 - RNA, 23 nt, homo sapiens
aacauucauugcugucggugggu

SEQ ID NO: 106 - RNA, 24 nt, homo sapiens
aacauucaaccugucggugaguuu

SEQ ID NO: 107 - RNA, 23 nt, homo sapiens
aacauucauuguugucggugggu

SEQ ID NO: 108 - RNA, 24 nt, homo sapiens
uuuggcaaugguagaacucacacu

SEQ ID NO: 109 - RNA, 22 nt, homo sapiens
uauggcacugguagaauucacu

SEQ ID NO: 110 - RNA, 22 nt, homo sapiens
uggacggagaacugauaagggu

SEQ ID NO: 111 - RNA, 22 nt, homo sapiens
uggagagaaaggcaguuccuga

SEQ ID NO: 112 - RNA, 22 nt, homo sapiens
caaagaauucuccuuuugggcu

SEQ ID NO: 113 - RNA, 22 nt, homo sapiens
ucgugucuuguguugcagccgg

SEQ ID NO: 114 - RNA, 22 nt, homo sapiens
caucccuugcaugguggagggu

SEQ ID NO: 115 - RNA, 23 nt, homo sapiens
uaaggugcaucuagugcagauag

SEQ ID NO: 116 - RNA, 21 nt, homo sapiens
uaaggugcaucuagugcaguu

SEQ ID NO: 117 - RNA, 21 nt, homo sapiens
cggcggggacggcgauugguc

SEQ ID NO: 118 - RNA, 23 nt, homo sapiens
ugauauguuugauauauuagguu

SEQ ID NO: 119 - RNA, 22 nt, homo sapiens
ugauauguuugauauuggguug

SEQ ID NO: 120 - RNA, 22 nt, homo sapiens
caacggaaucccaaaagcagcu

SEQ ID NO: 121 - RNA, 21 nt, homo sapiens
ccaguccugugccugccgccu

SEQ ID NO: 122 - RNA, 23 nt, homo sapiens
ugaguaccgccaugucuguuggg

SEQ ID NO: 123 - RNA, 21 nt, homo sapiens
ugcucauugcaugggcugugu

SEQ ID NO: 124 - RNA, 23 nt, homo sapiens
cccugugcccggcccacuucugc

SEQ ID NO: 125 - RNA, 21 nt, homo sapiens
cugaccuaugaauugacagcc

SEQ ID NO: 126 - RNA, 22 nt, homo sapiens
aacuggccuacaaagucccagu

SEQ ID NO: 127 - RNA, 22 nt, homo sapiens
aacuggcccucaaagucccgcu

SEQ ID NO: 128 - RNA, 22 nt, homo sapiens
uguaacagcaacuccaugugga

SEQ ID NO: 129 - RNA, 22 nt, homo sapiens
uagcagcacagaaauauuggca

SEQ ID NO: 130 - RNA, 22 nt, homo sapiens
uagguaguuucauguuguuggg

SEQ ID NO: 131 - RNA, 22 nt, homo sapiens
uagguaguuuccuguuguuggg

SEQ ID NO: 132 - RNA, 22 nt, homo sapiens
uucaccaccuucuccacccagc

SEQ ID NO: 133 - RNA, 21 nt, homo sapiens
acaguagucugcacauugguu

SEQ ID NO: 134 - RNA, 21 nt, homo sapiens
acaguagucugcacauugguu

SEQ ID NO: 135 - RNA, 23 nt, homo sapiens
ugugcaaaucuaugcaaaacuga

SEQ ID NO: 136 - RNA, 23 nt, homo sapiens
ugugcaaauccaugcaaaacuga

SEQ ID NO: 137 - RNA, 23 nt, homo sapiens
uaacacugucugguaacgauguu

SEQ ID NO: 138 - RNA, 22 nt, homo sapiens
uaauacugccugguaaugauga

SEQ ID NO: 139 - RNA, 23 nt, homo sapiens
uaauacugccggguaaugaugga

SEQ ID NO: 140 - RNA, 20 nt, homo sapiens
agagguauagggcaugggaa

SEQ ID NO: 141 - RNA, 22 nt, homo sapiens
gugaaauguuuaggaccacuag

SEQ ID NO: 142 - RNA, 22 nt, homo sapiens
uucccuuugucauccuaugccu

SEQ ID NO: 143 - RNA, 23 nt, homo sapiens
uccuucauuccaccggagucugu

SEQ ID NO: 144 - RNA, 22 nt, homo sapiens
uggaauguaaggaagugugugg

SEQ ID NO: 145 - RNA, 22 nt, homo sapiens
auaagacgagcaaaaagcuugu

SEQ ID NO: 146 - RNA, 22 nt, homo sapiens
auaagacgaacaaaagguuugu

SEQ ID NO: 147 - RNA, 23 nt, homo sapiens
uaaagugcuuauagugcagguag

SEQ ID NO: 148 - RNA, 23 nt, homo sapiens
caaagugcucauagugcagguag

SEQ ID NO: 149 - RNA, 23 nt, homo sapiens
uagcuuaucagacugauguugac

SEQ ID NO: 150 - RNA, 22 nt, homo sapiens
cugugcgugugacagcggcuga

SEQ ID NO: 151 - RNA, 22 nt, homo sapiens
uucccuuugucauccuucgccu

SEQ ID NO: 152 - RNA, 23 nt, homo sapiens
uuggggaaacggccgcugaguga

SEQ ID NO: 153 - RNA, 22 nt, homo sapiens
uagucccuuccuugaagcgguc

SEQ ID NO: 154 - RNA, 22 nt, homo sapiens
agcuuccaugacuccugaugga

SEQ ID NO: 155 - RNA, 21 nt, homo sapiens
uccucccaugccaagaacucc

SEQ ID NO: 156 - RNA, 23 nt, homo sapiens
accuuggcucuagacugcuuacu

SEQ ID NO: 157 - RNA, 22 nt, homo sapiens
ugccugucuacacuugcugugc

SEQ ID NO: 158 - RNA, 22 nt, homo sapiens
augaccuaugaauugacagaca

SEQ ID NO: 159 - RNA, 22 nt, homo sapiens
uaaucucagcuggcaacuguga

SEQ ID NO: 160 - RNA, 22 nt, homo sapiens
aaaucucugcaggcaaauguga

SEQ ID NO: 161 - RNA, 23 nt, homo sapiens
uacugcaucaggaacugauugga

SEQ ID NO: 162 - RNA, 21 nt, homo sapiens
uugugcuugaucuaaccaugu

SEQ ID NO: 163 - RNA, 21 nt, homo sapiens
ugauuguccaaacgcaauucu

SEQ ID NO: 164 - RNA, 22 nt, homo sapiens
agaauuguggcuggacaucugu

SEQ ID NO: 165 - RNA, 22 nt, homo sapiens
aagcugccaguugaagaacugu

SEQ ID NO: 166 - RNA, 22 nt, homo sapiens
agcuacauugucugcuggguuu

SEQ ID NO: 167 - RNA, 23 nt, homo sapiens
agcuacaucuggcuacugggucu

SEQ ID NO: 168 - RNA, 22 nt, homo sapiens
ugucaguuugucaaauacccca

SEQ ID NO: 169 - RNA, 22 nt, homo sapiens
caagucacuagugguuccguuu

SEQ ID NO: 170 - RNA, 22 nt, homo sapiens
gcccucugucaccuugcagacg

SEQ ID NO: 171 - RNA, 23 nt, homo sapiens
agcgcgggcugagcgcugccagu

SEQ ID NO: 172 - RNA, 22 nt, homo sapiens
gagagcaguguguguugccugg

SEQ ID NO: 173 - RNA, 22 nt, homo sapiens
auccccagauacaauggacaau

SEQ ID NO: 174 - RNA, 22 nt, homo sapiens
aucacauugccagggauuucca

SEQ ID NO: 175 - RNA, 21 nt, homo sapiens
aucacauugccagggauuacc

SEQ ID NO: 176 - RNA, 22 nt, homo sapiens
uggcucaguucagcaggaacag

SEQ ID NO: 177 - RNA, 22 nt, homo sapiens
cauugcacuugucucggucuga

SEQ ID NO: 178 - RNA, 22 nt, homo sapiens
uucaaguaauccaggauaggcu

SEQ ID NO: 179 - RNA, 22 nt, homo sapiens
uucaaguaauucaggauagguu

SEQ ID NO: 180 - RNA, 21 nt, homo sapiens
uucacaguggcuaaguuccgc

SEQ ID NO: 181 - RNA, 21 nt, homo sapiens
uucacaguggcuaaguucugc

SEQ ID NO: 182 - RNA, 22 nt, homo sapiens
aaggagcucacagucuauugag

SEQ ID NO: 183 - RNA, 22 nt, homo sapiens
gaggguuggguggaggcucucc

SEQ ID NO: 184 - RNA, 22 nt, homo sapiens
ugguuuaccgucccacauacau

SEQ ID NO: 185 - RNA, 22 nt, homo sapiens
uagcaccaucugaaaucgguua

SEQ ID NO: 186 - RNA, 23 nt, homo sapiens
uagcaccauuugaaaucaguguu

SEQ ID NO: 187 - RNA, 21 nt, homo sapiens
uagcaccauuugaaaucgguu

SEQ ID NO: 188 - RNA, 23 nt, homo sapiens
cagugcaauaguauugucaaagc

SEQ ID NO: 189 - RNA, 23 nt, homo sapiens
cagugcaaugauauugucaaagc

SEQ ID NO: 190 - RNA, 22 nt, homo sapiens
uaaacguggauguacuugcuuu

SEQ ID NO: 191 - RNA, 23 nt, homo sapiens
uaagugcuuccauguuuuaguag

SEQ ID NO: 192 - RNA, 22 nt, homo sapiens
aagugcuuccauguuucagugg

SEQ ID NO: 193 - RNA, 23 nt, homo sapiens
uaagugcuuccauguuugagugu

SEQ ID NO: 194 - RNA, 23 nt, homo sapiens
ucaacaaaaucacugaugcugga

SEQ ID NO: 195 - RNA, 22 nt, homo sapiens
guuccugcugaacugagccagu

SEQ ID NO: 196 - RNA, 24 nt, homo sapiens
uguaaacauccucgacuggaagcu

SEQ ID NO: 197 - RNA, 22 nt, homo sapiens
uguaaacauccuacacucagcu

SEQ ID NO: 198 - RNA, 24 nt, homo sapiens
uguaaacauccuacacucucagcu

SEQ ID NO: 199 - RNA, 24 nt, homo sapiens
uguaaacauccccgacuggaagcu

SEQ ID NO: 200 - RNA, 24 nt, homo sapiens
uguaaacauccuugacuggaagcu

SEQ ID NO: 201 - RNA, 23 nt, homo sapiens
ugcuaugccaacauauugccauc

SEQ ID NO: 202 - RNA, 20 nt, homo sapiens
auauggguuuacuaguuggu

SEQ ID NO: 203 - RNA, 22 nt, homo sapiens
auaggacucauauagugccagg

SEQ ID NO: 204 - RNA, 21 nt, homo sapiens
cacagcaaguguagacaggca

SEQ ID NO: 205 - RNA, 21 nt, homo sapiens
uucgcgggcgaaggcaaaguc

SEQ ID NO: 206 - RNA, 22 nt, homo sapiens
ugagggacagaugccagaagca

SEQ ID NO: 207 - RNA, 23 nt, homo sapiens
aucagggcuuguggaaugggaag

SEQ ID NO: 208 - RNA, 22 nt, homo sapiens
aaacuaaucucuacacugcugc

SEQ ID NO: 209 - RNA, 21 nt, homo sapiens
gcugcaccggagacuggguaa

SEQ ID NO: 210 - RNA, 22 nt, homo sapiens
cugacugaauagguagggucau

SEQ ID NO: 211 - RNA, 21 nt, homo sapiens
acagugagguagagggagugc

SEQ ID NO: 212 - RNA, 23 nt, homo sapiens
uaggagcucaacagaugccuguu

SEQ ID NO: 213 - RNA, 21 nt, homo sapiens
agcuuuugggaauucagguag

SEQ ID NO: 214 - RNA, 22 nt, homo sapiens
auaacauuguaaagcgcuucuu

SEQ ID NO: 215 - RNA, 21 nt, homo sapiens
auauaccuguucggucucuuu

SEQ ID NO: 216 - RNA, 22 nt, homo sapiens
aacuccaaacacucaaaacuca

SEQ ID NO: 217 - RNA, 23 nt, homo sapiens
caugcuaggauagaaagaauggg

SEQ ID NO: 218 - RNA, 23 nt, homo sapiens
uuuguauggauauguguguguau

SEQ ID NO: 219 - RNA, 22 nt, homo sapiens
caaccucgacgaucuccucagc

SEQ ID NO: 220 - RNA, 21 nt, homo sapiens
gguggggcaaugggaucaggu

SEQ ID NO: 221 - RNA, 22 nt, homo sapiens
auugccucuguucuaacacaag

SEQ ID NO: 222 - RNA, 22 nt, homo sapiens
ccucccacugcagagccugggg

SEQ ID NO: 223 - RNA, 22 nt, homo sapiens
uucagccaggcuagugcagucu

SEQ ID NO: 224 - RNA, 22 nt, homo sapiens
aagggcuuccucucugcaggac

SEQ ID NO: 225 - RNA, 22 nt, homo sapiens
cugggguucugagacagacagu

SEQ ID NO: 226 - RNA, 22 nt, homo sapiens
uauauagauuccauaaaucuau

SEQ ID NO: 227 - RNA, 22 nt, homo sapiens
ugcccugccuguuuucuccuuu

SEQ ID NO: 228 - RNA, 22 nt, homo sapiens
uagugaguuagagaugcagagc

SEQ ID NO: 229 - RNA, 22 nt, homo sapiens
cggggagagaacgcagugacgu

SEQ ID NO: 230 - RNA, 21 nt, homo sapiens
acuggccugggacuaccgggg

SEQ ID NO: 231 - RNA, 21 nt, homo sapiens
ugcacggcacuggggacacgu

SEQ ID NO: 232 - RNA, 22 nt, homo sapiens
gccucucucggagucgcucgga

SEQ ID NO: 233 - RNA, 20 nt, homo sapiens
uuggccauggggcugcgcgg

SEQ ID NO: 234 - RNA, 22 nt, homo sapiens
cccuugggucugaugggguagc

SEQ ID NO: 235 - RNA, 22 nt, homo sapiens
uccugcguaggaucugaggagu

SEQ ID NO: 236 - RNA, 22 nt, homo sapiens
ggccagccaccaggagggcugc

SEQ ID NO: 237 - RNA, 22 nt, homo sapiens
guggaguccuggggaauggaga

SEQ ID NO: 238 - RNA, 21 nt, homo sapiens
uauugcacauuacuaaguugc

SEQ ID NO: 239 - RNA, 22 nt, homo sapiens
aaaagcuggguugagagggcga

SEQ ID NO: 240 - RNA, 23 nt, homo sapiens
caccuugcgcuacucaggucugc

SEQ ID NO: 241 - RNA, 22 nt, homo sapiens
gcacauuacacggucgaccucu

SEQ ID NO: 242 - RNA, 21 nt, homo sapiens
cccaauacacggucgaccucu

SEQ ID NO: 243 - RNA, 23 nt, homo sapiens
cgcauccccuagggcauuggugu

SEQ ID NO: 244 - RNA, 22 nt, homo sapiens
uuuauugaggaccuccuaucaa

SEQ ID NO: 245 - RNA, 20 nt, homo sapiens
ccucugggcccuuccuccag

SEQ ID NO: 246 - RNA, 22 nt, homo sapiens
cuggcccucucugcccuuccgu

SEQ ID NO: 247 - RNA, 22 nt, homo sapiens
aacacaccugguuaaccucuuu

SEQ ID NO: 248 - RNA, 23 nt, homo sapiens
gcaaagcacacggccugcagaga

SEQ ID NO: 249 - RNA, 20 nt, homo sapiens
gccccugggccuauccuaga

SEQ ID NO: 250 - RNA, 22 nt, homo sapiens
ucaagagcaauaacgaaaaaug

SEQ ID NO: 251 - RNA, 21 nt, homo sapiens
uccuauaugaugccuuucuuc

SEQ ID NO: 252 - RNA, 21 nt, homo sapiens
uccagcaucagugauuuuguu

SEQ ID NO: 253 - RNA, 23 nt, homo sapiens
ucccuguccuccaggagcucacg

SEQ ID NO: 254 - RNA, 20 nt, homo sapiens
gugcauuguaguugcauugc

SEQ ID NO: 255 - RNA, 20 nt, homo sapiens
gugcauugcuguugcauugc

SEQ ID NO: 256 - RNA, 22 nt, homo sapiens
uuauaaagcaaugagacugauu

SEQ ID NO: 257 - RNA, 23 nt, homo sapiens
ucucacacagaaaucgcacccgu

SEQ ID NO: 258 - RNA, 21 nt, homo sapiens
gcugacuccuaguccagggcu

SEQ ID NO: 259 - RNA, 23 nt, homo sapiens
ugucugcccgcaugccugccucu

SEQ ID NO: 260 - RNA, 22 nt, homo sapiens
uggcagugucuuagcugguugu

SEQ ID NO: 261 - RNA, 23 nt, homo sapiens
aggcagugucauuagcugauugu

SEQ ID NO: 262 - RNA, 23 nt, homo sapiens
aggcaguguaguuagcugauugc

SEQ ID NO: 263 - RNA, 21 nt, homo sapiens
ccuccguguuaccuguccucu

SEQ ID NO: 264 - RNA, 22 nt, homo sapiens
uuaucagaaucuccagggguac

SEQ ID NO: 265 - RNA, 20 nt, homo sapiens
uugugaagaaagaaauucuu

SEQ ID NO: 266 - RNA, 22 nt, homo sapiens
aggaggcaucuugagaaaugga

SEQ ID NO: 267 - RNA, 22 nt, homo sapiens
uguuguacuuuuuuuuuuguuc

SEQ ID NO: 268 - RNA, 22 nt, homo sapiens
uagccuucagaucuugguguuu

SEQ ID NO: 269 - RNA, 22 nt, homo sapiens
aaagacauaguugcaagauggg

SEQ ID NO: 270 - RNA, 21 nt, homo sapiens
ucagcaggcaggcuggugcag

SEQ ID NO: 271 - RNA, 24 nt, homo sapiens
aauccuuggaaccuaggugugagu

SEQ ID NO: 272 - RNA, 22 nt, homo sapiens
caggcacgggagcucaggugag

SEQ ID NO: 273 - RNA, 22 nt, homo sapiens
aauugcacgguauccaucugua

SEQ ID NO: 274 - RNA, 22 nt, homo sapiens
uaaugccccuaaaaauccuuau

SEQ ID NO: 275 - RNA, 22 nt, homo sapiens
uugugucccauuauuggugauu

SEQ ID NO: 276 - RNA, 22 nt, homo sapiens
ugaguguugucuacgagggcau

SEQ ID NO: 277 - RNA, 22 nt, homo sapiens
aacucugucuucacucaugagu

SEQ ID NO: 278 - RNA, 22 nt, homo sapiens
accuuccucuccaugggucuuu

SEQ ID NO: 279 - RNA, 22 nt, homo sapiens
aauugcacuuuagcaaugguga

SEQ ID NO: 280 - RNA, 22 nt, homo sapiens
cucgugggcucuggccacggcc

SEQ ID NO: 281 - RNA, 19 nt, homo sapiens
ugaggauauggcagggaag

SEQ ID NO: 282 - RNA, 21 nt, homo sapiens
acucacucacaggauugugca

SEQ ID NO: 283 - RNA, 22 nt, homo sapiens
uaguggaugaugcacucugugc

SEQ ID NO: 284 - RNA, 21 nt, homo sapiens
ugaugauacagguggagguag

SEQ ID NO: 285 - RNA, 22 nt, homo sapiens
uauggaaagacuuugccacucu

SEQ ID NO: 286 - RNA, 21 nt, homo sapiens
aauaauacaugguugaucuuu

SEQ ID NO: 287 - RNA, 22 nt, homo sapiens
uaguggaugauggagacucggu

SEQ ID NO: 288 - RNA, 22 nt, homo sapiens
gccugcugggguggaaccuggu

SEQ ID NO: 289 - RNA, 21 nt, homo sapiens
acucaaacugugggggcacuu

SEQ ID NO: 290 - RNA, 23 nt, homo sapiens
aaagugcugcgacauuugagcgu

SEQ ID NO: 291 - RNA, 23 nt, homo sapiens
gaagugcuucgauuuuggggugu

SEQ ID NO: 292 - RNA, 22 nt, homo sapiens
uuauaauacaaccugauaagug

SEQ ID NO: 293 - RNA, 22 nt, homo sapiens
auauaauacaaccugcuaagug

SEQ ID NO: 294 - RNA, 22 nt, homo sapiens
uuuguucguucggcucgcguga

SEQ ID NO: 295 - RNA, 21 nt, homo sapiens
aucauagaggaaaauccacgu

SEQ ID NO: 296 - RNA, 21 nt, homo sapiens
aucauagaggaaaauccaugu

SEQ ID NO: 297 - RNA, 21 nt, homo sapiens
aacauagaggaaauuccacgu

SEQ ID NO: 298 - RNA, 22 nt, homo sapiens
aucacacaaaggcaacuuuugu

SEQ ID NO: 299 - RNA, 22 nt, homo sapiens
acuggacuuggagucagaaggc

SEQ ID NO: 300 - RNA, 21 nt, homo sapiens
ugguagacuauggaacguagg

SEQ ID NO: 301 - RNA, 21 nt, homo sapiens
uauguaauaugguccacaucu

SEQ ID NO: 302 - RNA, 22 nt, homo sapiens
uauacaagggcaagcucucugu

SEQ ID NO: 303 - RNA, 22 nt, homo sapiens
gaaguuguucgugguggauucg

SEQ ID NO: 304 - RNA, 22 nt, homo sapiens
agaucagaaggugauuguggcu

SEQ ID NO: 305 - RNA, 22 nt, homo sapiens
auuccuagaaauuguucauaau

SEQ ID NO: 306 - RNA, 22 nt, homo sapiens
uguccucuagggccugcagucu

SEQ ID NO: 307 - RNA, 21 nt, homo sapiens
aaaaggcauaaaaccaagaca

SEQ ID NO: 308 - RNA, 21 nt, homo sapiens
uaacgcauaauauggacaugu

SEQ ID NO: 309 - RNA, 22 nt, homo sapiens
uacugaguccuuuguucucuac

SEQ ID NO: 310 - RNA, 22 nt, homo sapiens
ugugggacuucuggccuugacu

SEQ ID NO: 311 - RNA, 22 nt, homo sapiens
ggaggaaccuuggagcuucggc

SEQ ID NO: 312 - RNA, 22 nt, homo sapiens
ucagguguggaaacugaggcag

SEQ ID NO: 313 - RNA, 22 nt, homo sapiens
aauucccuuguagauaacccgg

SEQ ID NO: 314 - RNA, 22 nt, homo sapiens
uacgcgcagaccacaggauguc

SEQ ID NO: 315 - RNA, 21 nt, homo sapiens
cagcccggaucccagcccacu

SEQ ID NO: 316 - RNA, 21 nt, homo sapiens
agcaauacuguuaccugaaau

SEQ ID NO: 317 - RNA, 21 nt, homo sapiens
ugugcagcaggccaaccgaga

SEQ ID NO: 318 - RNA, 22 nt, homo sapiens
gaauguugcucggugaaccccu

SEQ ID NO: 319 - RNA, 21 nt, homo sapiens
aauauaacacagauggccugu

SEQ ID NO: 320 - RNA, 22 nt, homo sapiens
auaguagaccguauagcguacg

SEQ ID NO: 321 - RNA, 20 nt, homo sapiens
uucaccugguccacuagccg

SEQ ID NO: 322 - RNA, 23 nt, homo sapiens
aucaacagacauuaauugggcgc

SEQ ID NO: 323 - RNA, 23 nt, homo sapiens
agcucggucugaggccccucagu

SEQ ID NO: 324 - RNA, 21 nt, homo sapiens
cagcagcaauucauguuuuga

SEQ ID NO: 325 - RNA, 25 nt, homo sapiens
aaugacacgaucacucccguugagu

SEQ ID NO: 326 - RNA, 22 nt, homo sapiens
uaauacugucugguaaaaccgu

SEQ ID NO: 327 - RNA, 21 nt, homo sapiens
ugucuugcaggccgucaugca

SEQ ID NO: 328 - RNA, 23 nt, homo sapiens
ucuuggaguaggucauugggugg

SEQ ID NO: 329 - RNA, 23 nt, homo sapiens
uguuccucugucucccagacucu

SEQ ID NO: 330 - RNA, 22 nt, homo sapiens
aucaugaugggcuccucggugu

SEQ ID NO: 331 - RNA, 21 nt, homo sapiens
uugcauauguaggauguccca

SEQ ID NO: 332 - RNA, 22 nt, homo sapiens
uggcaguguauuguuagcuggu

SEQ ID NO: 333 - RNA, 23 nt, homo sapiens
aggcaguguauuguuagcuggcu

SEQ ID NO: 334 - RNA, 22 nt, homo sapiens
caguugcuaguugcacuccucu

SEQ ID NO: 335 - RNA, 22 nt, homo sapiens
uuuugcgauguguuccuaauau

SEQ ID NO: 336 - RNA, 22 nt, homo sapiens
uuuugcaauauguuccugaaua

SEQ ID NO: 337 - RNA, 19 nt, homo sapiens
aaaccguuaccauuacuga

SEQ ID NO: 338 - RNA, 22 nt, homo sapiens
aacuguuugcagaggaaacuga

SEQ ID NO: 339 - RNA, 23 nt, homo sapiens
uagugcaauauugcuuauagggu

SEQ ID NO: 340 - RNA, 22 nt, homo sapiens
uaugugccuuuggacuacaucg

SEQ ID NO: 341 - RNA, 22 nt, homo sapiens
uguguugcauguguguauaugu

SEQ ID NO: 342 - RNA, 22 nt, homo sapiens
cacuccucuccucccgucuucu

SEQ ID NO: 343 - RNA, 22 nt, homo sapiens
ccggggggggcggggccucgcg

SEQ ID NO: 344 - RNA, 22 nt, homo sapiens
gucauacacggcucuccucucu

SEQ ID NO: 345 - RNA, 22 nt, homo sapiens
uccuguacugagcugccccgag

SEQ ID NO: 346 - RNA, 22 nt, homo sapiens
aaucauacagggacauccaguu

SEQ ID NO: 347 - RNA, 22 nt, homo sapiens
aaucguacagggucauccacuu

SEQ ID NO: 348 - RNA, 21 nt, homo sapiens
uugaaaggcuauuucuugguc

SEQ ID NO: 349 - RNA, 22 nt, homo sapiens
gugacaucacauauacggcagc

SEQ ID NO: 350 - RNA, 20 nt, homo sapiens
ccauggaucuccaggugggu

SEQ ID NO: 351 - RNA, 23 nt, homo sapiens
aguggggaacccuuccaugagga

SEQ ID NO: 352 - RNA, 22 nt, homo sapiens
uuguacaugguaggcuuucauu

SEQ ID NO: 353 - RNA, 23 nt, homo sapiens
ugaaacauacacgggaaaccucu

SEQ ID NO: 354 - RNA, 22 nt, homo sapiens
aaacaaacauggugcacuucuu

SEQ ID NO: 355 - RNA, 22 nt, homo sapiens
gguuguccaugguguguucauu

SEQ ID NO: 356 - RNA, 21 nt, homo sapiens
cagcagcacacugugguuugu

SEQ ID NO: 357 - RNA, 23 nt, homo sapiens
uuucaagccagggggcguuuuuc

SEQ ID NO: 358 - RNA, 21 nt, homo sapiens
uuaagacuugcagugauguuu

SEQ ID NO: 359 - RNA, 23 nt, homo sapiens
augcaccugggcaaggauucuga

SEQ ID NO: 360 - RNA, 23 nt, homo sapiens
uaauccuugcuaccugggugaga

SEQ ID NO: 361 - RNA, 22 nt, homo sapiens
aaugcacccgggcaaggauucu

SEQ ID NO: 362 - RNA, 22 nt, homo sapiens
aaugcaccugggcaaggauuca

SEQ ID NO: 363 - RNA, 23 nt, homo sapiens
uagcagcgggaacaguucugcag

SEQ ID NO: 364 - RNA, 21 nt, homo sapiens
gacccuggucugcacucuauc

SEQ ID NO: 365 - RNA, 22 nt, homo sapiens
cgucaacacuugcugguuuccu

SEQ ID NO: 366 - RNA, 22 nt, homo sapiens
guaaggcacccuucugaguaga

SEQ ID NO: 367 - RNA, 23 nt, homo sapiens
ugauuguagccuuuuggaguaga

SEQ ID NO: 368 - RNA, 23 nt, homo sapiens
ugauugguacgucuguggguaga

SEQ ID NO: 369 - RNA, 23 nt, homo sapiens
ugauugaaaccucuaagagugga

SEQ ID NO: 370 - RNA, 21 nt, homo sapiens
gugucuuuugcucugcaguca

SEQ ID NO: 371 - RNA, 22 nt, homo sapiens
aagugcugucauagcugagguc

SEQ ID NO: 372 - RNA, 23 nt, homo sapiens
uaaauuucaccuuucugagaagg

SEQ ID NO: 373 - RNA, 22 nt, homo sapiens
uucacaaggaggugucauuuau

SEQ ID NO: 374 - RNA, 22 nt, homo sapiens
uucucaaggaggugucguuuau

SEQ ID NO: 375 - RNA, 21 nt, homo sapiens
auugacacuucugugaguaga

SEQ ID NO: 376 - RNA, 22 nt, homo sapiens
uucucaagagggaggcaaucau

SEQ ID NO: 377 - RNA, 22 nt, homo sapiens
gagugccuucuuuuggagcguu

SEQ ID NO: 378 - RNA, 22 nt, homo sapiens
uucucgaggaaagaagcacuuu

SEQ ID NO: 379 - RNA, 24 nt, homo sapiens
aucuggagguaagaagcacuuucu

SEQ ID NO: 380 - RNA, 22 nt, homo sapiens
aucuggagguaagaagcacuuu

SEQ ID NO: 381 - RNA, 22 nt, homo sapiens
aucgugcaucccuuuagagugu

SEQ ID NO: 382 - RNA, 22 nt, homo sapiens
aucgugcauccuuuuagagugu

SEQ ID NO: 383 - RNA, 22 nt, homo sapiens
gaaagcgcuucccuuugcugga

SEQ ID NO: 384 - RNA, 22 nt, homo sapiens
caaagcgcuccccuuuagaggu

SEQ ID NO: 385 - RNA, 23 nt, homo sapiens
caaagcgcuucucuuuagagugu

SEQ ID NO: 386 - RNA, 22 nt, homo sapiens
caaagcgcuucccuuuggagcg

SEQ ID NO: 387 - RNA, 22 nt, homo sapiens
aaagcgcuucccuucagagugu

SEQ ID NO: 388 - RNA, 22 nt, homo sapiens
gaaagcgcuucucuuuagagga

SEQ ID NO: 389 - RNA, 22 nt, homo sapiens
aaagugcauccuuuuagagugu

SEQ ID NO: 390 - RNA, 22 nt, homo sapiens
aaagugcauccuuuuagagguu

SEQ ID NO: 391 - RNA, 22 nt, homo sapiens
aaagugcaucuuuuuagaggau

SEQ ID NO: 392 - RNA, 23 nt, homo sapiens
caaagugccucccuuuagagugu

SEQ ID NO: 393 - RNA, 22 nt, homo sapiens
uucuccaaaagggagcacuuuc

SEQ ID NO: 394 - RNA, 21 nt, homo sapiens
cuccagagggaaguacuuucu

SEQ ID NO: 395 - RNA, 22 nt, homo sapiens
aaagugcuuccuuuuagagggu

SEQ ID NO: 396 - RNA, 22 nt, homo sapiens
aaagugcuuccuuuuagagggu

SEQ ID NO: 397 - RNA, 22 nt, homo sapiens
aaagugcuucucuuuggugggu

SEQ ID NO: 398 - RNA, 22 nt, homo sapiens
aaagugcuuccuuuuugagggu

SEQ ID NO: 399 - RNA, 22 nt, homo sapiens
caagugcuuccuuuuagagggu

SEQ ID NO: 400 - RNA, 24 nt, homo sapiens
acaaagugcuucccuuuagagugu

SEQ ID NO: 401 - RNA, 22 nt, homo sapiens
aaagugcuucccuuuagaguua

SEQ ID NO: 402 - RNA, 22 nt, homo sapiens
aacgcacuucccuuuagagugu

SEQ ID NO: 403 - RNA, 22 nt, homo sapiens
aaaaugguucccuuuagagugu

SEQ ID NO: 404 - RNA, 22 nt, homo sapiens
aacgcgcuucccuauagagggu

SEQ ID NO: 405 - RNA, 22 nt, homo sapiens
cuacaaagggaagcacuuucuc

SEQ ID NO: 406 - RNA, 22 nt, homo sapiens
cuccagagggaugcacuuucuc

SEQ ID NO: 407 - RNA, 22 nt, homo sapiens
gaaagcgcuuccuuuuagagga

SEQ ID NO: 408 - RNA, 22 nt, homo sapiens
gaacaugcauccuuucagaggg

SEQ ID NO: 409 - RNA, 23 nt, homo sapiens
cucuugagggaagcacuuucugu

SEQ ID NO: 410 - RNA, 21 nt, homo sapiens
cugcaaagggaagcccuuucu

SEQ ID NO: 411 - RNA, 22 nt, homo sapiens
caugccuugaguguaggaccgu

SEQ ID NO: 412 - RNA, 22 nt, homo sapiens
aucauacaaggacaauuucuuu

SEQ ID NO: 413 - RNA, 22 nt, homo sapiens
uggugggcacagaaucuggacu

SEQ ID NO: 414 - RNA, 22 nt, homo sapiens
ugugacagauugauaacugaaa

SEQ ID NO: 415 - RNA, 22 nt, homo sapiens
aaacauucgcggugcacuucuu

SEQ ID NO: 416 - RNA, 21 nt, homo sapiens
ucuuguuaaaaagcagauucu

SEQ ID NO: 417 - RNA, 22 nt, homo sapiens
ucaguaaauguuuauuagauga

SEQ ID NO: 418 - RNA, 22 nt, homo sapiens
agcucauccauaguugucacug

SEQ ID NO: 419 - RNA, 22 nt, homo sapiens
ugucuuacucccucaggcacau

SEQ ID NO: 420 - RNA, 20 nt, homo sapiens
gcgacccacucuugguuucc

SEQ ID NO: 421 - RNA, 21 nt, homo sapiens
gcgacccauacuugguuucag

SEQ ID NO: 422 - RNA, 21 nt, homo sapiens
aacaggugacugguuagacaa

SEQ ID NO: 423 - RNA, 21 nt, homo sapiens
gaugagcucauuguaauauga

SEQ ID NO: 424 - RNA, 22 nt, homo sapiens
uuuggugcauauuuacuuuagg

SEQ ID NO: 425 - RNA, 22 nt, homo sapiens
aucaaggaucuuaaacuuugcc

SEQ ID NO: 426 - RNA, 22 nt, homo sapiens
cgaaaacagcaauuaccuuugc

SEQ ID NO: 427 - RNA, 22 nt, homo sapiens
cacgcucaugcacacacccaca

SEQ ID NO: 428 - RNA, 22 nt, homo sapiens
auucuaauuucuccacgucuuu

SEQ ID NO: 429 - RNA, 22 nt, homo sapiens
guagauaaaauauugguaccug

SEQ ID NO: 430 - RNA, 23 nt, homo sapiens
uucauuugguauaaaccgcgauu

SEQ ID NO: 431 - RNA, 22 nt, homo sapiens
uugagaaugaugaaucauuagg

SEQ ID NO: 432 - RNA, 21 nt, homo sapiens
ucuuguguucucuagaucagu

SEQ ID NO: 433 - RNA, 23 nt, homo sapiens
uuacaguuguucaaccaguuacu

SEQ ID NO: 434 - RNA, 21 nt, homo sapiens
uuaugguuugccugggacuga

SEQ ID NO: 435 - RNA, 22 nt, homo sapiens
ugggcguaucuguaugcuaggg

SEQ ID NO: 436 - RNA, 21 nt, homo sapiens
uuggccacaauggguuagaac

SEQ ID NO: 437 - RNA, 21 nt, homo sapiens
ugagaaccacgucugcucuga

SEQ ID NO: 438 - RNA, 22 nt, homo sapiens
gagcuuauucauaaaagugcag

SEQ ID NO: 439 - RNA, 22 nt, homo sapiens
uugugucaauaugcgaugaugu

SEQ ID NO: 440 - RNA, 22 nt, homo sapiens
ugugucacucgaugaccacugu

SEQ ID NO: 441 - RNA, 22 nt, homo sapiens
uacgucaucguugucaucguca

SEQ ID NO: 442 - RNA, 22 nt, homo sapiens
uuugauaagcugacaugggaca

SEQ ID NO: 443 - RNA, 22 nt, homo sapiens
agaaggcacuaugagauuuaga

SEQ ID NO: 444 - RNA, 21 nt, homo sapiens
ugagcuaaaugugugcuggga

SEQ ID NO: 445 - RNA, 21 nt, homo sapiens
uccgagccugggucucccucu

SEQ ID NO: 446 - RNA, 22 nt, homo sapiens
acucaaaacccuucagugacuu

SEQ ID NO: 447 - RNA, 23 nt, homo sapiens
aaacucuacuuguccuucugagu

SEQ ID NO: 448 - RNA, 21 nt, homo sapiens
uaguaccaguaccuuguguuc

SEQ ID NO: 449 - RNA, 22 nt, homo sapiens
gacuauagaacuuucccccuca

SEQ ID NO: 450 - RNA, 22 nt, homo sapiens
gugagucucuaagaaaagagga

SEQ ID NO: 451 - RNA, 22 nt, homo sapiens
augcugacauauuuacuagagg

SEQ ID NO: 452 - RNA, 22 nt, homo sapiens
uggguuuacguugggagaacuu

SEQ ID NO: 453 - RNA, 23 nt, homo sapiens
aaagacauaggauagagucaccu

SEQ ID NO: 454 - RNA, 21 nt, homo sapiens
agacacauuuggagagggaac

SEQ ID NO: 455 - RNA, 22 nt, homo sapiens
acuuguaugcuagcucagguag

SEQ ID NO: 456 - RNA, 22 nt, homo sapiens
uuuaggauaagcuugacuuuug

SEQ ID NO: 457 - RNA, 21 nt, homo sapiens
aauggcgccacuaggguugug

SEQ ID NO: 458 - RNA, 21 nt, homo sapiens
uucacuggaguuuguuucaau

SEQ ID NO: 459 - RNA, 20 nt, homo sapiens
uaugucugcugaccaucacc

SEQ ID NO: 460 - RNA, 22 nt, homo sapiens
auaauacaugguuaaccucuuu

SEQ ID NO: 461 - RNA, 21 nt, homo sapiens
aauauuauacagucaaccucu

SEQ ID NO: 462 - RNA, 22 nt, homo sapiens
aggaccuucccugaaccaagga

SEQ ID NO: 463 - RNA, 23 nt, homo sapiens
uacccauugcauaucggaguugu

SEQ ID NO: 464 - RNA, 22 nt, homo sapiens
accaggaggcugaggccccuca

SEQ ID NO: 465 - RNA, 21 nt, homo sapiens
ugucacucggcucggcccacu

SEQ ID NO: 466 - RNA, 23 nt, homo sapiens
uuuccucauauucauucaggagu

SEQ ID NO: 467 - RNA, 24 nt, homo sapiens
aggaagcccuggaggggcuggagg

SEQ ID NO: 468 - RNA, 22 nt, homo sapiens
cuguaugcccucaccgcucagc

SEQ ID NO: 469 - RNA, 21 nt, homo sapiens
cuguccuaagguuguugaguu

SEQ ID NO: 470 - RNA, 24 nt, homo sapiens
uggaagacuagugauuuuguuguu

SEQ ID NO: 471 - RNA, 22 nt, homo sapiens
aaggagcuuacaaucuagcugg

SEQ ID NO: 472 - RNA, 22 nt, homo sapiens
ugcggggcuagggcuaacagca

SEQ ID NO: 473 - RNA, 21 nt, homo sapiens
uuugugaccugguccacuaac

SEQ ID NO: 474 - RNA, 20 nt, homo sapiens
cggcucugggucugugggga

SEQ ID NO: 475 - RNA, 22 nt, homo sapiens
acuccagccccacagccucagc

SEQ ID NO: 476 - RNA, 24 nt, homo sapiens
ugcaccaugguugucugagcaugc

SEQ ID NO: 477 - RNA, 22 nt, homo sapiens
ugagaccucuggguucugagcu

SEQ ID NO: 478 - RNA, 21 nt, homo sapiens
uccaguaccacgugucagggc

SEQ ID NO: 479 - RNA, 21 nt, homo sapiens
gcaggaacuugugagucuccu

SEQ ID NO: 480 - RNA, 22 nt, homo sapiens
cugcccuggcccgagggaccga

SEQ ID NO: 481 - RNA, 21 nt, homo sapiens
ccuggaaacacugagguugug

SEQ ID NO: 482 - RNA, 21 nt, homo sapiens
uggauuucuuugugaaucacc

SEQ ID NO: 483 - RNA, 22 nt, homo sapiens
uccauuacacuacccugccucu

SEQ ID NO: 484 - RNA, 24 nt, homo sapiens
gugaacgggcgccaucccgaggcu

SEQ ID NO: 485 - RNA, 21 nt, homo sapiens
uacucaaaaagcugucaguca

SEQ ID NO: 486 - RNA, 21 nt, homo sapiens
uuaauaucggacaaccauugu

SEQ ID NO: 487 - RNA, 21 nt, homo sapiens
uacuuggaaaggcaucaguug

SEQ ID NO: 488 - RNA, 22 nt, homo sapiens
ugcaacgaaccugagccacuga

SEQ ID NO: 489 - RNA, 22 nt, homo sapiens
ugcaacuuaccugagucauuga

SEQ ID NO: 490 - RNA, 22 nt, homo sapiens
cacuguguccuuucugcguaga

SEQ ID NO: 491 - RNA, 22 nt, homo sapiens
cacuggcuccuuucuggguaga

SEQ ID NO: 492 - RNA, 23 nt, homo sapiens
ucuuugguuaucuagcuguauga

SEQ ID NO: 493 - RNA, 22 nt, homo sapiens
uauugcacuugucccggccugu

SEQ ID NO: 494 - RNA, 22 nt, homo sapiens
uauugcacucgucccggccucc

SEQ ID NO: 495 - RNA, 23 nt, homo sapiens
caaagugcuguucgugcagguag

SEQ ID NO: 496 - RNA, 22 nt, homo sapiens
ugucuacuacuggagacacugg

SEQ ID NO: 497 - RNA, 21 nt, homo sapiens
auccgcgcucugacucucugc

SEQ ID NO: 498 - RNA, 21 nt, homo sapiens
uucucuguuuuggccaugugu

SEQ ID NO: 499 - RNA, 22 nt, homo sapiens
aaauuauuguacaucggaugag

SEQ ID NO: 500 - RNA, 21 nt, homo sapiens
uucaacggguauuuauugagc

SEQ ID NO: 501 - RNA, 23 nt, homo sapiens
uuuggcacuagcacauuuuugcu

SEQ ID NO: 502 - RNA, 22 nt, homo sapiens
ugagguaguaaguuguauuguu

SEQ ID NO: 503 - RNA, 21 nt, homo sapiens
aacccguagauccgaucuugu

SEQ ID NO: 504 - RNA, 22 nt, homo sapiens
cacccguagaaccgaccuugcg

(19) United States
(12) Patent Application Publication
(10) Pub. No.: US 2016/0251720 A1
(43) Pub. Date: Sep. 1, 2016

(54) MicroRNA PROFILES IN HEART FAILURE: METHODS AND SYSTEMS FOR DETECTION AND USE

(71) Applicants: THE TRUSTEES OF COLUMBIA UNIVERSITY IN THE CITY NEW YORK, New York, NY (US); ROCKEFELLER UNIVERSITY, New York, NY (US)

(72) Inventors: Paul Christian Schulze, New York, NY (US); Thomas Tuschl, New York, NY (US); Kemal Akat, New York, NY (US)

(21) Appl. No.: 15/033,481

(22) PCT Filed: Nov. 3, 2014
(86) PCT No.: PCT/US14/63690

§ 371 Date: Apr. 29, 2016

Related U.S. Application Data
(60) Provisional application No. 61/898,588, filed on Nov. 1, 2013.
(60) Provisional application No. 62/000,977, filed on May 20, 2014.

(51) Int. Cl. C12Q 1/68; C12N 15/113

(52) U.S. Cl. CPC C12Q 1/6883; C12N 15/113; C12Q 2600/178; C12Q 2600/106; C12Q 2600/118; C12Q 2600/16; C12N 2320/31; C12N 2310/11; A61M 1/122

(57) ABSTRACT

The level of miRNAs in a sample from a patient is assayed and used as an indicator of the efficacy of a therapeutic intervention for a cardiovascular disease, such as heart failure. The levels of a plurality of miRNAs, such as myomirs, may be measured. Based on the measured level of the miRNAs, the therapeutic intervention may be modified, adjusted, continued or discontinued. The miRNA level may also be used to assess the severity or disease progression of a cardiovascular disease.

### CROSS REFERENCE TO RELATED APPLICATION

This application claims priority to U.S. Provisional Application No. 61/898,588 (filed on Nov. 1, 2013) and 62/000,977 (filed on May 20, 2014), which are incorporated herein by reference in their entirety.

### STATEMENT REGARDING FEDERALLY SPONSORED RESEARCH OR DEVELOPMENT

This invention was made with government support under the NIH Common Fund, NIH Grant Nos. K23HL095742, P30HL101272, UL1RR024156, HL073029, HD068546, and 8UL1TR000043 awarded by the National Institutes of Health. The government may have certain rights in the invention.

### FIELD OF THE INVENTION

The present invention relates to the detection of microRNAs for evaluating or monitoring the efficacy of a therapeutic intervention for cardiovascular diseases, or for assessing disease progression of heart failure in a patient.

### BACKGROUND OF THE INVENTION

Heart failure (HF) is associated with high morbidity as well as significant mortality. There has been an increased incidence of the disease worldwide. The clinical syndrome of heart failure is the result of heterogeneous myocardial or vascular diseases, and is defined by insufficiency to maintain blood circulation throughout the body. Despite significant advances in the clinical management of HF, conventional therapies are ultimately ineffective in many patients who progress to advanced HF. In these cases, implantation of left ventricular assist devices (LVAD) and/or heart transplantation can be the only viable options.

It is difficult to determine the precise etiology of heart failure, a factor impeding the development of more specific therapies. Furthermore, there is a general lack of diagnostic techniques at the molecular level. While protein biomarkers have been established for diagnostic and prognostic evaluation of patients with HF, there is currently no systematic assessment of RNA biomarkers that allow for rapid diagnosis and potential treatment.

MicroRNAs (miRNAs or miRs) are a class of regulatory RNAs that post-transcriptionally regulate gene expression. MiRNAs are evolutionarily conserved, small non-coding RNA molecules of approximately 18 to 25 nucleotides in length. Weiland et al. (2012) RNA Biol 9(6):850-859. Bartel D P (2009) Cell 136(2):215-233. Each miRNA is able to downregulate hundreds of target mRNAs comprising partially complementary sequences to the miRNAs. MiRNAs act as repressors of target mRNAs by promoting their degradation, or by inhibiting translation Braun et al. (2013) Adv Exp Med Biol 768:147-163.

MicroRNAs are promising targets for drug and biomarker development Weiland et al. (2012) RNA Biol 9(6):850-859. Target recognition requires base pairing of the miRNA 5′ end nucleotides (seed sequence) to complementary target mRNA regions located typically within the 3′UTR. Bartel D P (2009) Cell 136(2):215-233. Additionally, the recent detection of miRNPs (ribonucleoproteins), which contain associated miRNAs, in body fluids points towards their potential value as biomarkers for tissue injury Laterza et al. (2009) Clin Chem 55:1977-1983; Ai et al. (2010) Biochem Biophys Res Commun 391:73-77. Additionally, it is also possible that miRNPs can act as paracrine and endocrine regulators of gene expression Valadi et al. (2007) Nat Cell Biol 9:654-659; Williams et al. (2013) Proc Natl Acad Sci USA 110:4255-4260.

The function of miRNAs has been widely studied in animal models of HF. The muscle-specific miR-1/206 and 133a/b, and the heart-specific 208a/b, and 499, also referred to as myomirs, were shown to contribute to muscle- or myocardial function van Rooij et al. (2007) Science 316:575-579; van Rooij et al. (2009) Dev Cell 17:662-673. MiRNAs have been profiled in failing human myocardium, and a selected subset were also investigated as circulating biomarkers in HF (Yang et al. (2007) Nat Med 13:486-491; Thum et al. (2007) Circulation 116:258-267; Ikeda et al. (2007) Physiol Genomics 31:367-373; Sucharov et al. (2008) J Mol Cell Cardiol 45:185-192; Matkovich et al. (2009) Circulation 119:1263-1271; Naga et al. (2009) J Biol Chem 284:27487-27499; Yang et al. (2014) Circulation 129:1009-1021; Leptidis et al. (2013) PLoS One 8:e57800); Tijsen et al. (2010) Circ Res 106:1035-1039; Goren et al. (2012) Eur J Heart Fail 14:147-154; Dickinson et al. (2013) Eur J Heart Fail 15:650-659; Corsten et al. (2010) Circ Cardiovasc Genet 3:499-506; Tutarel et al. (2013) Int J Cardiol 167:63-66; Fukushima et al. (2011) Circ J 75:336-340).

There is still a need for additional diagnostic markers to assist in evaluating the severity of cardiovascular diseases, such as heart failure, and to define the prognosis and the response to treatment.

### SUMMARY

The present invention provides for a method for identifying a subject in need of treatment for a cardiovascular disease. The method may comprise the steps of: (a) obtaining a sample from the subject (e.g., a plasma or serum sample, or any other samples as discussed herein); (b) assaying the levels of a plurality of miRNAs in the sample, wherein the plurality of miRNAs comprises 3 or more (or 4 or more, 5 or more, 6 or more, 7 or more, 8 or more, 9 or more, 10 or more, 15 or more, 20 or more, 25 or more, 30 or more, 35 or more, 3-504, 5-504, 10-504, 15-504, 20-504, 30-504, 50-100, 100-200, 200-300, or 300-400) miRNAs listed in Table 1 (SEQ ID NOs: 1-504), or in any of Tables 3-7; (c) comparing the levels obtained in step (b) with the levels of the plurality of miRNAs in a control sample; and (d) treating the subject for a cardiovascular disease, if the levels of at least 2 (or at least 3, at least 4, at least 5, at least 6, at least 7, at least 8, at least 9, at least 10, at least 20, at least 30, at least 40, at least 50, between 5 and 30, between 5 and 10, between 10 and 20, between 30 and 50, between 10 and 200, or between 50 and 100) miRNAs obtained in step (b) are at least 2 fold (or at least 5 fold, at least 10 fold, at least 15 fold, at least 20 fold, at least 50 fold, or at least 100 fold, etc.) of their levels in the control sample.

Also encompassed by the present invention is a method for assessing the efficacy of a therapy for a cardiovascular disease in a patient. The method may comprise the steps of: (a) obtaining a first sample (e.g., a plasma or serum sample, or any other samples as discussed herein) from the patient before initiation of the therapy; (b) assaying the levels of a plurality of miRNAs in the first sample, wherein the plurality of miRNAs comprise 3 or more (or 4 or more, 5 or more, 6 or more, 7 or more, 8 or more, 9 or more, 10 or more, 15 or more, 20 or more, 25 or more, 30 or more, 35 or more, 3-504, 5-504, 10-504, 15-504, 20-504, 30-504, 50-100, 100-200, 200-300, or 300-400) miRNAs listed in Table 1 (SEQ ID NOs: 1-504), or in any of Tables 3-7; (c) obtaining a second sample (e.g., a plasma or serum sample, or any other samples as discussed herein) from the patient after initiation of the therapy; (d) assaying the levels of the plurality of miRNAs in the second sample; and (e) comparing the levels of step (b) with the levels of step (d). If the levels of at least 2 (or at least 3, at least 4, at least 5, at least 6, at least 7, at least 8, at least 9, at least 10, at least 20, at least 30, at least 40, at least 50, between 5 and 30, between 5 and 10, between 10 and 20, between 30 and 50, between 10 and 200, or between 50 and 100) miRNAs obtained in step (d) are less than about 80% (or less than about 70%, less than about 60%, less than about 50%, less than about 40%, less than about 30%, less than about 20%, less than about 10%, less than about 5%, less than about 2%, or less than about 1%) of their levels obtained in step (b), the therapy is effective. The therapy may be continued if the levels of at least two miRNAs obtained in step (d) are less than about 10% (or less than about 80% (or less than about 70%, less than about 60%, less than about 50%, less than about 40%, less than about 30%, or less than about 20%) of their levels obtained in step (b).

Also encompassed by the present invention is a method for evaluating a cardiovascular disease or monitoring progression of a cardiovascular disease in a patient, the method comprising the steps of: (a) obtaining a sample (e.g., a plasma or serum sample, or any other samples as discussed herein) from the patient; (b) testing the sample for levels of a plurality of miRNAs, wherein the plurality of miRNAs comprises three or more miRNAs listed in Table 1 (SEQ ID NOs: 1-504), or in any of Tables 3-7; and (c) comparing the levels of step (b) with the levels of the plurality of miRNAs in a control sample.

The miRNAs with the level changes can be any combination of two or more miRNAs selected from the group consisting of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-203 and miR-126. For example, the miRNAs can be selected from the group consisting of miR-208a, miR-208b, miR-499 or mixtures thereof. The miRNAs with the level changes can also be any combination of two or more miRNAs selected from the group consisting of miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320. The miRNAs may comprise two or more myomirs.

The present invention provides for a method for assessing efficacy of a therapy for a cardiovascular disease in a patient, the method comprising the steps of: (a) obtaining a first sample (e.g., a plasma or serum sample, or any other samples as discussed herein) from the patient before initiation of the therapy; (b) assaying the levels of a plurality of miRNAs in the first sample, wherein the plurality of miRNAs comprises three or more miRNAs selected from the group consisting of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126, miR-203, miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320; (c) obtaining a second sample (e.g., a plasma or serum sample, or any other samples as discussed herein) from the patient after initiation of therapy; (d) testing the second sample for levels of the plurality of microRNAs; and (e) comparing the levels of step (b) with the levels of step (d).

The present invention also provides for a method for evaluating a cardiovascular disease or monitoring progression of a cardiovascular disease in a patient, the method comprising the steps of: (a) obtaining a sample from the patient; (b) assaying the levels of a plurality of miRNAs in the sample, wherein the plurality of miRNAs comprises three or more miRNAs selected from the group consisting of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126, miR-203, miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320; and (c) comparing the levels of step (b) with the levels of the plurality of miRNAs in a control sample.

The cardiovascular disease may be heart failure, such as advanced or stable heart failure.

The subject may be treated with (the therapy may be) a pharmacologic composition, a medical device, surgery, or any combination thereof. For example, the medical device can be a left ventricular assist device (LVAD), treated for, e.g., at least 3 months, at least about 6 months, about 3 months, about 6 months, about 2 months to about 3 years, about 3 months to about 2 years, about 6 months to about 1 year, about 1 month to about 5 years, or longer.

The subject can be treated with (the therapy may be) antisense oligonucleotides targeting at least one miRNA selected from the group consisting of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-203 and miR-126. For example, the antisense oligonucleotide may target one or more miRNA selected from the group consisting of miR-208a, miR-208b, miR-499 or mixtures thereof. The subject can be treated with antisense oligonucleotides targeting at least one miRNA selected from the group consisting of miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320. The miRNAs may comprise two or more myomirs.

The control sample may be from a healthy subject or a plurality of healthy subjects.

The levels of the plurality of microRNA may be determined by RNA sequencing, microarray profiling or real-time PCR.

The present invention also provides for a kit comprising miRNA-specific primers for reverse transcribing or amplifying 3 or more (or 4 or more, 5 or more, 6 or more, 7 or more, 8 or more, 9 or more, 10 or more, 15 or more, 20 or more, 25 or more, 30 or more, 35 or more, 3-504, 5-504, 10-504, 15-504, 20-504, 30-504, 50-100, 100-200, 200-300, or 300-400) miRNAs selected from Table 1, or selected from any of Tables 3-7, in a plasma or serum sample from a patient receiving treatment for a cardiovascular disease; and instructions for measuring the 3 or more miRNAs for evaluating or monitoring the efficacy of a therapeutic intervention for treating a cardiovascular disease in the patient.

The present invention provides for a kit comprising miRNA-specific primers for reverse transcribing or amplifying 3 or more (or 4 or more, 5 or more, 6 or more, 7 or more, 8 or more, 9 or more, 10 or more, 15 or more, 20 or more, 25 or more, 30 or more, 35 or more, 3-504, 5-504, 10-504, 15-504, 20-504, 30-504, 50-100, 100-200, 200-300, or 300-400) miRNAs selected from Table 1, or selected from any of Tables 3-7, in a plasma or serum sample from a subject who may be in need of treatment for a cardiovascular disease; and instructions for measuring the 3 or more miRNAs for evaluating or identifying a need to treat a cardiovascular disease in the subject.

The miRNA-specific primers may be for miRNAs selected from miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126 and miR-203. For example, the miRNA-specific primers may be for miRNAs selected from the group consisting of miR-208a, miR-208b, miR-499 or mixtures thereof. The miRNA-specific primers may be for miRNAs selected from miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320.

The kit may additionally contain a labeled-nucleic acid probe specific for each miRNA of the kit.

### BRIEF DESCRIPTION OF THE FIGURES

FIG. 1 shows, for Example 1, the number of individuals in each group and tissue with the number of samples shown in parentheses.

FIG. 2 shows grouping of miRNA deep-sequencing reads based on the principles of genomic organization and sequence homology by myomir example.

FIG. 3 shows circulating miRNA dynamics in heart failure.

### DETAILED DESCRIPTION

The methods of the present invention assay the levels of miRNAs in a plasma or serum sample taken from a patient having a cardiovascular disease or from a subject suspected of having a cardiovascular disease. The levels of miRNAs in the sample can be used as an indicator of the efficacy of a therapeutic intervention for treating a cardiovascular disease, or for assessing the severity or disease progression of a cardiovascular disease, such as heart failure. A plurality of miRNAs, such as myomirs, may be measured. Based on the levels of the miRNAs, a subject may be diagnosed with a cardiovascular disease, and then treated with a therapy for the disease. For patients under any therapy, based on the miRNA levels, the therapeutic intervention may be continued when it is effective, or altered, if ineffective.

The present methods can identify a subject in need of treatment for a cardiovascular disease. The method may contain the following steps: (a) obtaining a sample (e.g., a plasma or serum sample, or other samples as discussed herein) from the subject; (b) assaying the levels of a plurality of miRNAs in the sample; (c) comparing the levels obtained in step (b) with the levels of the plurality of miRNAs in a control sample; and (d) treating the subject for a cardiovascular disease, if the levels of at least 2 (or at least 3, at least 4, at least 5, at least 6, at least 7, at least 8, at least 9, at least 10, at least 20, at least 30, at least 40, at least 50, between 5 and 30, between 5 and 10, between 10 and 20, between 30 and 50, or between 50 and 100) miRNAs obtained in step (b) are at least 1.1 fold, at least 1.2 fold, at least 1.3 fold, at least 1.4 fold, at least 1.5 fold, at least 1.6 fold, at least 1.8 fold, at least 2 fold, at least 5 fold, at least 10 fold, at least 15 fold, at least 20 fold, at least 50 fold, at least 100 fold, at least 120 fold, from about 2 fold to about 500 fold, from about 1.1 fold to about 10 fold, from about 1.1 fold to about 5 fold, from about 1.5 fold to about 5 fold, from about 2 fold to about 5 fold, from about 5 fold to about 10 fold, from about 5 fold to about 200 fold, from about 10 fold to about 150 fold, from about 10 fold to about 20 fold, from about 20 fold to about 150 fold, from about 20 fold to about 50 fold, from about 30 fold to about 150 fold, from about 50 fold to about 100 fold, from about 70 fold to about 150 fold, from about 100 fold to about 150 fold, from about 10 fold to about 100 fold, from about 100 fold to about 200 fold, about 10% (alternatively referred to as about 10 fold decrease, or “−10” fold change as the format shown in Tables 3-7) to about 90% (i.e., about 1.1 fold decrease, or “−1.1” fold change), about 12.5% (i.e., about 8 fold decrease, or “−8” fold change) to about 80% (i.e., about 1.25 fold decrease, or “−1.25” fold change), about 20% (i.e., about 5 fold decrease, or “−5” fold change) to about 70% (i.e., about 1.5 fold decrease, or “−1.5” fold change), about 25% (i.e., about 4 fold decrease, or “−4” fold change) to about 60% (i.e., about 1.7 fold decrease, or “−1.7” fold change), or about 25% (i.e., about 4 fold decrease, or “−4” fold change) to about 50% (i.e., about 2 fold decrease, or “−2” fold change) of their levels in the control sample. The control sample may be from a healthy subject or a plurality of healthy subjects. In certain embodiments, the plurality of miRNAs comprises 2 or more, 3 or more, 4 or more, 5 or more, 6 or more, 7 or more, 8 or more, 9 or more, 10 or more, 15 or more, 20 or more, 25 or more, 30 or more, 35 or more, 3-504, 5-504, 10-504, 15-504, 20-504, 30-504, 50-100, 100-200, 200-300, or 300-400 miRNAs listed in Table 1 (SEQ ID NOs: 1-504). In another embodiment, the plurality of miRNAs comprises 2 or more, 3 or more, 4 or more, 5 or more, 6 or more, 7 or more, 8 or more, 9 or more, 10 or more, 15 or more, 20 or more, 25 or more, 30 or more, 35 or more, 3-504, 5-504, 10-504, 15-504, 20-504, 30-504, 50-100, 100-200, 200-300, or 300-400 miRNAs listed in any of Tables 3-7. The two or more miRNAs with increased or decreased levels in the sample compared to a control sample can be any combination of two or more miRNAs selected from miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126, and miR-203. The two or more miRNAs with increased or decreased levels in the sample compared to a control sample can be any combination of two or more miRNAs selected from miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320.

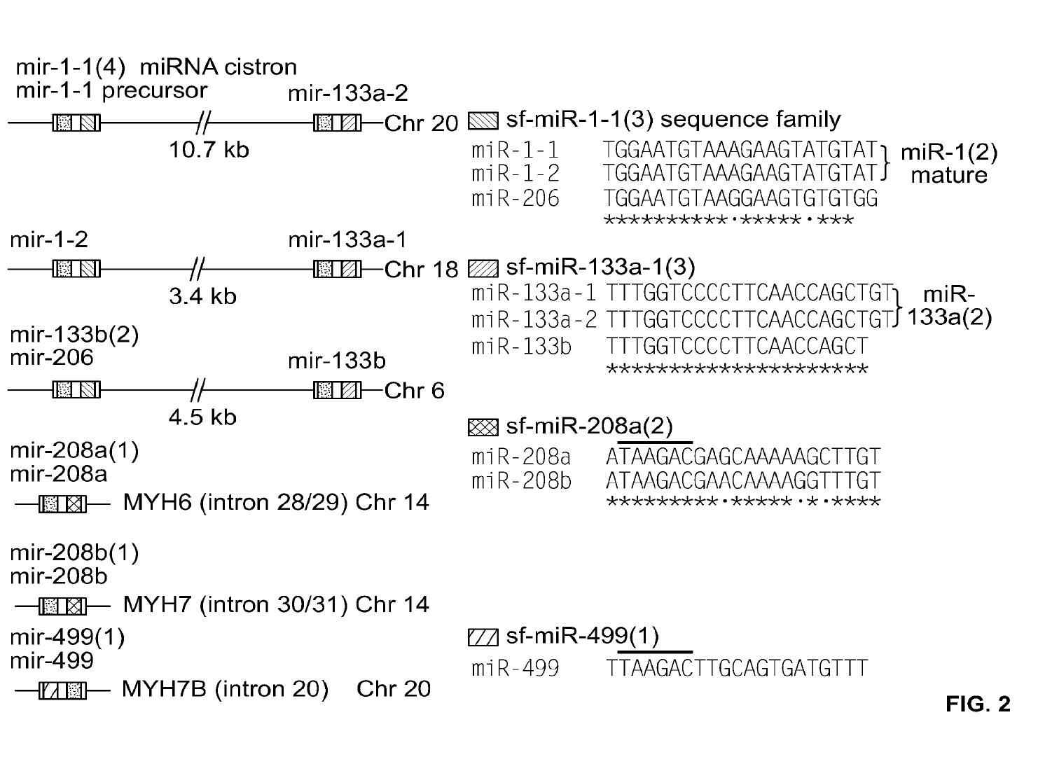

FIG. 2 illustrates grouping of miRNA deep-sequencing reads based on the principles of genomic organization and sequence homology by myomir example. The outlined boxes show miRNA stem-loop precursors with patterned rectangles representing guide strands for the different miRNA sequence family (sf); rectangles with dotted patterns depict star strands. The miR-1-1(3) and miR-133a-1(3) family members are organized in two-member cistrons that are expressed under the control of their own promoters. The mir-208a/b(1) and mir-499(1) cistrons are located in the introns of the myosin genes MYH6, MYH7, and MYH7B, respectively, and are excised from the pre-mRNA. sRNAseq (small RNA sequencing) reads may be reported either by (i) matching mature sequences (e.g., miR-1(2)) with the number in parentheses indicating the number of genes encoding identical mature sequences, by (ii) reads matching miRNAs belonging to a cistron (e.g., mir-1-1(4)), the number in parentheses indicating mature sequences encoded in that cistron; or by (iii) reads matching miRNA sf members (e.g., sf-miR-1-1(3)), with the number in parentheses indicating mature reads with identical bases in positions 2-7 and at maximum 50% mismatch in the remaining sequence. Asterisks and dots indicate similarities and differences in the alignments, respectively. The black solid bar marks the identical seed sequence of the families sf-miR-208a(2) and sf-miR-499(1).

When diagnosed, the subject may be treated with a pharmacologic composition, a medical device, e.g., a left ventricular assist device (LVAD), and/or surgery.

Also encompassed by the present invention is a method for assessing efficacy of a therapy for a cardiovascular disease in a patient. The method may contain the following steps: (a) obtaining a first sample from the patient before initiation of the therapy; (b) assaying the levels of a plurality of miRNAs in the first sample; (c) obtaining a second sample from the patient after initiation of the therapy; (d) assaying the levels of the plurality of miRNAs in the second sample; (e) comparing the levels of step (b) with the levels of step (d). If the levels of at least 2 (at least 3, at least 4, at least 5, at least 6, at least 7, at least 8, at least 9, at least 10, at least 20, at least 30, at least 40, at least 50, between 5 and 30, between 5 and 10, between 10 and 20, between 30 and 50, or between 50 and 100) miRNAs obtained in step (d) are less than about 70% (alternatively referred to as about 1.4 fold decrease, or “−1.4” fold change as the format shown in Tables 3-7), less than about 60% (alternatively referred to as about 1.7 fold decrease, or “−1.7” fold change), less than about 50% (alternatively referred to as about 2 fold decrease, or “−2” fold change), less than about 40% (alternatively referred to as about 2.5 fold decrease, or “−2.5” fold change), less than about 30% (alternatively referred to as about 3.3 fold decrease, or “−3.3” fold change), less than about 20% (alternatively referred to as about 5 fold decrease, or “−5” fold change), less than about 10% (alternatively referred to as about 10 fold decrease, or “−10” fold change), less than about 5% (alternatively referred to as about 20 fold decrease, or “−20” fold change), less than about 2% (alternatively referred to as about 50 fold decrease, or “−50” fold change), less than about 1% (alternatively referred to as about 100 fold decrease, or “−100” fold change), less than about 0.5% (alternatively referred to as about 200 fold decrease, or “−200” fold change), about 1% to about 70%, about 5% to about 60%, about 10% to about 50%, about 15% to about 40%, about 5% to about 20%, about 1% to about 20%, about 10% to about 30%, at least 1.1 fold, at least 1.2 fold, at least 1.3 fold, at least 1.4 fold, at least 1.5 fold, at least 1.6 fold, at least 1.8 fold, at least 2 fold, at least 5 fold, at least 10 fold, at least 15 fold, at least 20 fold, at least 50 fold, at least 100 fold, at least 120 fold, from about 2 fold to about 500 fold, from about 1.1 fold to about 10 fold, from about 1.1 fold to about 5 fold, from about 1.5 fold to about 5 fold, from about 2 fold to about 5 fold, from about 5 fold to about 10 fold, from about 5 fold to about 200 fold, from about 10 fold to about 150 fold, from about 10 fold to about 20 fold, from about 20 fold to about 150 fold, from about 20 fold to about 50 fold, from about 30 fold to about 150 fold, from about 50 fold to about 100 fold, from about 70 fold to about 150 fold, from about 100 fold to about 150 fold, from about 10 fold to about 100 fold, from about 100 fold to about 200 fold, of their levels obtained in step (b), the therapy is considered to be effective. An effective therapy may be continued, or discontinued if the patient's condition has improved and is no longer in need of treatment. An ineffective treatment may be altered or modified, or replaced with other treatment. In certain embodiments, the plurality of miRNAs comprises 2 or more, 3 or more, 4 or more, 5 or more, 6 or more, 7 or more, 8 or more, 9 or more, 10 or more, 15 or more, 20 or more, 25 or more, 30 or more, 35 or more, 3-504, 5-504, 10-504, 15-504, 20-504, 30-504, 50-100, 100-200, 200-300, or 300-400 miRNAs listed in Table 1 (SEQ ID NOs: 1-504).). In another embodiment, the plurality of miRNAs comprises 2 or more, 3 or more, 4 or more, 5 or more, 6 or more, 7 or more, 8 or more, 9 or more, 10 or more, 15 or more, 20 or more, 25 or more, 30 or more, 35 or more, 3-504, 5-504, 10-504, 15-504, 20-504, 30-504, 50-100, 100-200, 200-300, or 300-400 miRNAs listed in any of Tables 3-7. The two or more miRNAs with decreased or increased levels in the second sample compared to the first sample can be any combination of two or more miRNAs selected from miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-203, and miR-126. The two or more miRNAs with decreased or increased levels in the sample compared to a control sample can be any combination of two or more miRNAs selected from miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320.

The present methods can include the steps of measuring the level of at least one miRNA in a sample from a patient receiving a therapeutic intervention, and comparing the measured level to a reference level or the level of at least one miRNA in a control sample. The measured level of the at least one miRNA is indicative of the therapeutic efficacy of the therapeutic intervention.

The therapeutic interventions may be a pharmacologic intervention, devices, surgical intervention, or any combination thereof. For example, implantation of an LVAD, antisense oligonucleotides targeting miR-208a or miR-208b or other miRNA species, and/or a conventional therapy, such as angiotensin-converting enzyme (ACE) inhibitor, may be used to treat the cardiovascular disease. Based on the measured miRNAs levels, therapy may be continued or altered, e.g., by change of dose or dosing frequency, or by addition of other active agents, or change of therapeutic regimen altogether. In certain embodiments, the treatment is implantation of an LVAD, and the level of a combination of markers listed in Table 1 is monitored during treatment.

The present invention also encompasses a method of predicting or assessing the level of severity of heart failure or heart failure progression in a patient. The methods of the present invention may also be used to detect the specific stage of heart failure. In one embodiment, the method comprises measuring the level of at least one miRNA selected from Table 1, or selected from any of Tables 3-7, in a biological sample from a patient; and comparing the measured level to a reference level or the level of said at least one miRNA in a control sample, wherein the measured level of said at least one miRNA is indicative of the level of severity of heart failure or heart failure progression in the patient. In other embodiments, an increase or decrease in the level of the miRNA is indicative of the level of severity of heart failure or heart failure progression in the patient.

In other embodiments, an increase in the measured level of the miRNA relative to the level of the miRNA in the control sample or pre-determined reference value is indicative of the level of severity of heart failure or heart failure progression in the patient. For instance, in such embodiments, when the levels of 2 or more, 3 or more, 4 or more, 5 or more, 6 or more, 7 or more, 8 or more, 9 or more, 10 or more, about 2 to about 10, about 3 to about 9, or about 4 to about 8 miRNAs selected from the group comprising miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126 and miR-203 are increased (or decreased) when compared to the levels in a control sample or pre-determined reference value, the increase (or decrease) is indicative of the level of severity of heart failure or heart failure progression in the patient. In another embodiment, when the levels of 2 or more, 3 or more, 4 or more, 5 or more, 6 or more, 7 or more, 8 or more, 9 or more, 10 or more, about 2 to about 10, about 3 to about 9, or about 4 to about 8 miRNAs selected from the group comprising miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320 are increased or decreased when compared to the levels in a control sample or pre-determined reference value, the increase or decrease is indicative of the level of severity of heart failure or heart failure progression in the patient.

In other embodiments, a reduction or decrease in the measured level of the miRNA relative to the level of the miRNA in the control sample (e.g., a sample obtained from a healthy, age-matched subject, a sample obtained from a subject not suffering from or diagnosed with heart failure, or a sample obtained from the same subject a period of time ago when he/she was free of any cardiovascular disease) or pre-determined reference value is indicative of the level of severity of heart failure or heart failure progression in the patient. For instance, in such embodiments, when the level of two or more miRNAs selected from the group comprising miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126 and miR-203 is decreased (or increased) when compared to the level in a control sample or pre-determined reference value, the decrease (or increase) is indicative of the level of severity of heart failure or heart failure progression in the patient. In another embodiment, when the levels of two or more miRNAs selected from the group comprising miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320 are decreased (or increased) when compared to the levels in a control sample or pre-determined reference value, the decrease (or increase) is indicative of the level of severity of heart failure or heart failure progression in the patient.

The methods and systems of the present invention may be used to identify patients at risk for cardiovascular disease such as heart failure, stage patients for heart failure, e.g., Class I-IV, determine types of therapeutic intervention, e.g., pharmacological, mechanical or surgical, or identify compounds that could treat cardiovascular disease by modulating microRNA levels either in vitro or in vivo.

The expression profile of the miRNAs in patients having various stages of heart failure may be determined. The expression profile of the patients with heart failure may be compared with a reference value, where the reference value is based on a set of miRNA expression profiles in unaffected individuals or with the patients before, after and during therapy. The changes in miRNA expression may be used to alter or direct therapy, including, but not limited to, initiating, altering or stopping therapy.

Another aspect of the invention is a kit containing a reagent for measuring at least one miRNA in a biological sample, instructions for measuring at least one miRNA and instructions for evaluating or monitoring the efficacy of a therapeutic intervention for treating a cardiovascular disease in a patient based on the level of the at least one miRNA. In some embodiments, the kit contains reagents for measuring from 2 to about 20 human miRNAs, including 1, 2, 3, 4, 5, 6, 7, 8, 9, 10, 11, 12, 13, 14, 15, 16, 17, 18, 19, 20 up to n from Table 1, or from any of Tables 3-7. Also encompassed by the invention are kits for assessing or predicting the severity or progression of a cardiovascular disease, e.g., heart failure, in a subject may comprise a reagent for measuring at least one miRNA in a biological sample and instructions for assessing cardiovascular disease severity or progression based on the level of the at least one miRNA.

TABLE 1
miRNA Sequences
The term “hsa” leading each miRNA name
indicates that the miRNA is a human sequence.

| SEQ | | |
|---|---|---|
| ID | | Mature miRNA Sequence |
| NO: | miRNA | (5′ to 3′) |
| 1 | hsa-let-7a | UGAGGUAGUAGGUUGUAUAGUU |
| 2 | hsa-let-7b | UGAGGUAGUAGGUUGUGUGGUU |
| 3 | hsa-let-7c | UGAGGUAGUAGGUUGUAUGGUU |
| 4 | hsa-let-7d | AGAGGUAGUAGGUUGCAUAGUU |
| 5 | hsa-let-7e | UGAGGUAGGAGGUUGUAUAGUU |
| 6 | hsa-let-7f | UGAGGUAGUAGGUUGUAUGGUU |
| 7 | hsa-let-7g | UGAGGUAGUAGUUUGUACAGUU |
| 8 | hsa-let-7i | UGAGGUAGUAGUUUGUGCUGUU |
| 9 | hsa-miR-1 | UGGAAUGUAAAGAAGUAUGUAU |
| 10 | hsa-miR-100 | AACCCGUAGAUCCGAACUUGUG |
| 11 | hsa-miR-101 | UACAGUACUGUGAUAACUGAAG |
| 12 | hsa-miR-103 | AGCAGCAUUGUACAGGGCUAUGA |
| 13 | hsa-miR-105 | UCAAAUGCUCAGACUCCUGUGGU |
| 14 | hsa-miR-106a | AAAAGUGCUUACAGUGCAGGUAG |
| 15 | hsa-miR-106b | UAAAGUGCUGACAGUGCAGAU |
| 16 | hsa-miR-107 | AGCAGCAUUGUACAGGGCUAU |
| 17 | hsa-miR-10a | UACCCUGUAGAUCCGAAUUUGU |
| 18 | hsa-miR-10b | UACCCUGUAGAACCGAAUUUGU |
| 19 | hsa-miR-1179 | AAGCAUUCUUUCAUUGGUUGGU |
| 20 | hsa-miR-1180 | UUUCCGGCUCGCGUGGGUGUGU |
| 21 | hsa-miR-1185-5p | AGAGGAUACCCUUUGUAUGUUC |
| 22 | hsa-miR-1193-5p | GGGAUGGUAGACCGGUGACGUGC |
| 23 | hsa-miR-1197 | UAGGACACAUGGUCUACUUCU |
| 24 | hsa-miR-122 | UGGAGUGUGACAAUGGUGUUUGU |
| 25 | hsa-miR-124 | UAAGGCACGCGGUGAAUGCCA |
| 26 | hsa-miR-1245-3p | AAGUGAUCUAAAGGCCUACAU |
| 27 | hsa-miR-1247 | ACCCGUCCCGUUCGUCCCCGGA |
| 28 | hsa-miR-1249 | ACGCCCUUCCCCCCCUUCUUCA |
| 29 | hsa-miR-1250 | ACGGUGCUGGAUGUGGCCUUU |
| 30 | hsa-miR-1251 | ACUCUAGCUGCCAAAGGCGCU |
| 31 | hsa-miR-1252 | AGAAGGAAAUUGAAUUCAUUU |
| 32 | hsa-miR-1255a-5p | AGGAUGAGCAAAGAAAGUAGAUU |
| 33 | hsa-miR-1255b | CGGAUGAGCAAAGAAAGUGGUU |
| 34 | hsa-miR-1256-3p | CUAAAGAGAAGUCAAUGCAUGA |
| 35 | hsa-miR-1258-3p | AGUUAGGAUUAGGUCGUGGAA |
| 36 | hsa-miR-125a | UCCCUGAGACCCUUUAACCUGU |
| 37 | hsa-miR-125b | UCCCUGAGACCCUAACUUGUGA |
| 38 | hsa-miR-126 | UCGUACCGUGAGUAAUAAUGCG |
| 39 | hsa-miR-1263-5p | AUGGUACCCUGGCAUACUGAGU |
| 40 | hsa-miR-1264 | CAAGUCUUAUUUGAGCACCUGU |
| 41 | hsa-miR-1266 | CCUCAGGGCUGUAGAACAGGGCU |
| 42 | hsa-miR-1269 | CUGGACUGAGCCGUGCUACUGG |
| 43 | hsa-miR-127-3p | UCGGAUCCGUCUGAGCUUGGCU |
| 44 | hsa-miR-1270 | CUGGAGAUAUGGAAGAGCUGUGU |
| 45 | hsa-miR-1271 | CUUGGCACCUAGCAAGCACUCA |
| 46 | hsa-miR-1277-3p | UACGUAGAUAUAUAUGUAUUUU |
| 47 | hsa-miR-1278 | UAGUACUGUGCAUAUCAUCUAU |
| 48 | hsa-miR-128 | UCACAGUGAACCGGUCUCUUU |
| 49 | hsa-miR-1283-5p | UCUACAAAGGAAAGCGCUUUCU |
| 50 | hsa-miR-1284-3p | GAAAGCCCAUGUUUGUAUUGGA |
| 51 | hsa-miR-1286 | UGCAGGACCAAGAUGAGCCCU |
| 52 | hsa-miR-1287 | UGCUGGAUCAGUGGUUCGAGU |
| 53 | hsa-miR-1289-1-3p | UGGAGUCCAGGAAUCUGCAUUU |
| 54 | hsa-miR-129-1-3p | AAGCCCUUACCCCAAAAAGUAU |
| 55 | hsa-miR-129-2-3p | AAGCCCUUACCCCAAAAAGCAU |
| 56 | hsa-miR-1293-5p | UCUGGGUGGUCUGGAGAUUUGU |
| 57 | hsa-miR-1294-5p | UGUGAGGUUGGCAUUGUUGUCU |
| 58 | hsa-miR-1295 | UUAGGCCGCAGAUCUGGGUGA |
| 59 | hsa-miR-1296 | UUAGGGCCCUGGCUCCAUCUCC |
| 60 | hsa-miR-1298-5p | UUCAUUCGGCUGUCCAGAUG |
| 61 | hsa-miR-1301 | UUGCAGCUGCCUGGGAGUGACUUC |
| 62 | hsa-miR-1303-3p | UUUUAGAGACGGGGUCUUGCUCU |
| 63 | hsa-miR-1304-5p | CGGUUUGAGGCUACAGUGAGAU |
| 64 | hsa-miR-1305 | UUUUCAACUCUAAUGGGAGAGA |
| 65 | hsa-miR-1306-5p | CCACCUCCCCUGCAAACGUCCA |
| 66 | hsa-miR-1307 | UCGACCGGACCUCGACCGGCU |
| 67 | hsa-miR-130a | CAGUGCAAUGUUAAAAGGGCAU |
| 68 | hsa-miR-130b-3p | CAGUGCAAUGAUGAAAGGGCAU |
| 69 | hsa-miR-132-3p | UAACAGUCUACAGCCAUGGUCG |
| 70 | hsa-miR-1323 | UCAAAACUGAGGGGCAUUUUCU |
| 71 | hsa-miR-133a | UUUGGUCCCCUUCAACCAGCUGU |
| 72 | hsa-miR-133b | UUUGGUCCCCUUCAACCAGCU |
| 73 | hsa-miR-134 | UGUGACUGGUUGACCAGAGGGG |
| 74 | hsa-miR-135a | UAUGGCUUUUUAUUCCUAUGUGA |
| 75 | hsa-miR-135b | UAUGGCUUUUCAUUCCUAUGUGA |
| 76 | hsa-miR-136-5p | ACUCCAUUUGUUUUGAUGAUGGA |
| 77 | hsa-miR-137 | UUAUUGCUUAAGAAUACGCGUAG |
| 78 | hsa-miR-138 | AGCUGGUGUUGUGAAUCAGGCCG |
| 79 | hsa-miR-139 | UCUACAGUGCACGUGUCUCCAGU |
| 80 | hsa-miR-140-3p | ACCACAGGGUAGAACCACGGAC |
| 81 | hsa-miR-141 | UAACACUGUCUGGUAAAGAUGGC |
| 82 | hsa-miR-142-3p | UGUAGUGUUUCCUACUUUAUGGA |
| 83 | hsa-miR-143 | UGAGAUGAAGCACUGUAGCUC |
| 84 | hsa-miR-144 | UACAGUAUAGAUGAUGUACU |
| 85 | hsa-miR-145 | GUCCAGUUUUCCCAGGAAUCCCU |
| 86 | hsa-miR-1468 | CUCCGUUUGCCUGUUUCGCUGA |
| 87 | hsa-miR-146a | UGAGAACUGAAUUCCAUGGGUU |
| 88 | hsa-miR-146b | UGAGAACUGAAUUCCAUAGGCU |
| 89 | hsa-miR-147 | GUGUGCGGAAAUGCUUCUGCU |
| 90 | hsa-miR-148a | UCAGUGCACUACAGAACUUUGU |
| 91 | hsa-miR-148b | UCAGUGCAUCACAGAACUUUGU |
| 92 | hsa-miR-149 | UCUGGCUCCGUGUCUUCACUCCC |
| 93 | hsa-miR-150 | UCUCCCAACCCUUGUACCAGUG |
| 94 | hsa-miR-151-5p | UCGAGGAGCUCACAGUCUAGU |
| 95 | hsa-miR-152 | UCAGUGCAUGACAGAACUUGG |
| 96 | hsa-miR-153 | UUGCAUAGUCACAAAAGUGAUC |
| 97 | hsa-miR-1537 | AAAACCGUCUAGUUACAGUUGU |
| 98 | hsa-miR-154-3p | AAUCAUACACGGUUGACCUAUU |
| 99 | hsa-miR-155 | UUAAUGCUAAUCGUGAUAGGGGU |
| 100 | hsa-miR-15a | UAGCAGCACAUAAUGGUUUGU |
| 101 | hsa-miR-15b | UAGCAGCACAUCAUGGUUUACA |
| 102 | hsa-miR-16 | UAGCAGCACGUAAAUAUUGGCG |
| 103 | hsa-miR-17 | CAAAGUGCUUACAGUGCAGGUAG |
| 104 | hsa-miR-181a | AACAUUCAACGCUGUCGGUGAGU |
| 105 | hsa-miR-181b | AACAUUCAUUGCUGUCGGUGGGU |
| 106 | hsa-miR-181c | AACAUUCAACCUGUCGGUGAGUUU |
| 107 | hsa-miR-181d | AACAUUCAUUGUUGUCGGUGGGU |
| 108 | hsa-miR-182 | UUUGGCAAUGGUAGAACUCACACU |
| 109 | hsa-miR-183 | UAUGGCACUGGUAGAAUUCACU |
| 110 | hsa-miR-184 | UGGACGGAGAACUGAUAAGGGU |
| 111 | hsa-miR-185 | UGGAGAGAAAGGCAGUUCCUGA |
| 112 | hsa-miR-186 | CAAAGAAUUCUCCUUUUGGGCU |
| 113 | hsa-miR-187 | UCGUGUCUUGUGUUGCAGCCGG |
| 114 | hsa-miR-188 | CAUCCCUUGCAUGGUGGAGGGU |
| 115 | hsa-miR-18a | UAAGGUGCAUCUAGUGCAGAUAG |
| 116 | hsa-miR-18b | UAAGGUGCAUCUAGUGCAGUU |
| 117 | hsa-miR-1908 | CGGCGGGGACGGCGAUUGGUC |
| 118 | hsa-miR-190a | UGAUAUGUUUGAUAUAUUAGGUU |
| 119 | hsa-miR-190b | UGAUAUGUUUGAUAUUGGGUUG |
| 120 | hsa-miR-191 | CAACGGAAUCCCAAAAGCAGCU |
| 121 | hsa-miR-1910 | CCAGUCCUGUGCCUGCCGCCU |
| 122 | hsa-miR-1911 | UGAGUACCGCCAUGUCUGUUGGG |
| 123 | hsa-miR-1912-5p | UGCUCAUUGCAUGGGCUGUGU |
| 124 | hsa-miR-1914-5p | CCCUGUGCCCGGCCCACUUCUGC |
| 125 | hsa-miR-192 | CUGACCUAUGAAUUGACAGCC |
| 126 | hsa-miR-193a-3p | AACUGGCCUACAAAGUCCCAGU |
| 127 | hsa-miR-193b | AACUGGCCCUCAAAGUCCCGCU |
| 128 | hsa-miR-194 | UGUAACAGCAACUCCAUGUGGA |
| 129 | hsa-miR-195 | UAGCAGCACAGAAAUAUUGGCA |
| 130 | hsa-miR-196a | UAGGUAGUUUCAUGUUGUUGGG |
| 131 | hsa-miR-196b | UAGGUAGUUUCCUGUUGUUGGG |
| 132 | hsa-miR-197 | UUCACCACCUUCUCCACCCAGC |
| 133 | hsa-miR-199a-3p | ACAGUAGUCUGCACAUUGGUU |
| 134 | hsa-miR-199b-3p | ACAGUAGUCUGCACAUUGGUU |
| 135 | hsa-miR-19a | UGUGCAAAUCUAUGCAAAACUGA |
| 136 | hsa-miR-19b | UGUGCAAAUCCAUGCAAAACUGA |
| 137 | hsa-miR-200a | UAACACUGUCUGGUAACGAUGUU |
| 138 | hsa-miR-200b | UAAUACUGCCUGGUAAUGAUGA |
| 139 | hsa-miR-200c | UAAUACUGCCGGGUAAUGAUGGA |
| 140 | hsa-miR-202-3p | AGAGGUAUAGGGCAUGGGAA |
| 141 | hsa-miR-203 | GUGAAAUGUUUAGGACCACUAG |
| 142 | hsa-miR-204 | UUCCCUUUGUCAUCCUAUGCCU |
| 143 | hsa-miR-205 | UCCUUCAUUCCACCGGAGUCUGU |
| 144 | hsa-miR-206 | UGGAAUGUAAGGAAGUGUGUGG |
| 145 | hsa-miR-208a | AUAAGACGAGCAAAAAGCUUGU |
| 146 | hsa-miR-208b | AUAAGACGAACAAAAGGUUUGU |
| 147 | hsa-miR-20a | UAAAGUGCUUAUAGUGCAGGUAG |
| 148 | hsa-miR-20b | CAAAGUGCUCAUAGUGCAGGUAG |
| 149 | hsa-miR-21 | UAGCUUAUCAGACUGAUGUUGAC |
| 150 | hsa-miR-210 | CUGUGCGUGUGACAGCGGCUGA |
| 151 | hsa-miR-211 | UUCCCUUUGUCAUCCUUCGCCU |
| 152 | hsa-miR-2110 | UUGGGGAAACGGCCGCUGAGUGA |
| 153 | hsa-miR-2114 | UAGUCCCUUCCUUGAAGCGGUC |
| 154 | hsa-miR-2115 | AGCUUCCAUGACUCCUGAUGGA |
| 155 | hsa-miR-2116-3p | UCCUCCCAUGCCAAGAACUCC |
| 156 | hsa-miR-212-5p | ACCUUGGCUCUAGACUGCUUACU |
| 157 | hsa-miR-214-5p | UGCCUGUCUACACUUGCUGUGC |
| 158 | hsa-miR-215 | AUGACCUAUGAAUUGACAGACA |
| 159 | hsa-miR-216a | UAAUCUCAGCUGGCAACUGUGA |
| 160 | hsa-miR-216b | AAAUCUCUGCAGGCAAAUGUGA |
| 161 | hsa-miR-217 | UACUGCAUCAGGAACUGAUUGGA |
| 162 | hsa-miR-218 | UUGUGCUUGAUCUAACCAUGU |
| 163 | hsa-miR-219-1-5p | UGAUUGUCCAAACGCAAUUCU |
| 164 | hsa-miR-219-2-3p | AGAAUUGUGGCUGGACAUCUGU |
| 165 | hsa-miR-22 | AAGCUGCCAGUUGAAGAACUGU |
| 166 | hsa-miR-221 | AGCUACAUUGUCUGCUGGGUUU |
| 167 | hsa-miR-222 | AGCUACAUCUGGCUACUGGGUCU |
| 168 | hsa-miR-223 | UGUCAGUUUGUCAAAUACCCCA |
| 169 | hsa-miR-224 | CAAGUCACUAGUGGUUCCGUUU |
| 170 | hsa-miR-2276-5p | GCCCUCUGUCACCUUGCAGACG |
| 171 | hsa-miR-2277 | AGCGCGGGCUGAGCGCUGCCAGU |
| 172 | hsa-miR-2278 | GAGAGCAGUGUGUGUUGCCUGG |
| 173 | hsa-miR-2355-5p | AUCCCCAGAUACAAUGGACAAU |
| 174 | hsa-miR-23a | AUCACAUUGCCAGGGAUUUCCA |
| 175 | hsa-miR-23b | AUCACAUUGCCAGGGAUUACC |
| 176 | hsa-miR-24 | UGGCUCAGUUCAGCAGGAACAG |
| 177 | hsa-miR-25 | CAUUGCACUUGUCUCGGUCUGA |
| 178 | hsa-miR-26a | UUCAAGUAAUCCAGGAUAGGCU |
| 179 | hsa-miR-26b | UUCAAGUAAUUCAGGAUAGGUU |
| 180 | hsa-miR-27a | UUCACAGUGGCUAAGUUCCGC |
| 181 | hsa-miR-27b | UUCACAGUGGCUAAGUUCUGC |
| 182 | hsa-miR-28-5p | AAGGAGCUCACAGUCUAUUGAG |
| 183 | hsa-miR-296-3p | GAGGGUUGGGUGGAGGCUCUCC |
| 184 | hsa-miR-299-5p | UGGUUUACCGUCCCACAUACAU |
| 185 | hsa-miR-29a | UAGCACCAUCUGAAAUCGGUUA |
| 186 | hsa-miR-29b | UAGCACCAUUUGAAAUCAGUGUU |
| 187 | hsa-miR-29c | UAGCACCAUUUGAAAUCGGUU |
| 188 | hsa-miR-301a | CAGUGCAAUAGUAUUGUCAAAGC |
| 189 | hsa-miR-301b | CAGUGCAAUGAUAUUGUCAAAGC |
| 190 | hsa-miR-302a-5p | UAAACGUGGAUGUACUUGCUUU |
| 191 | hsa-miR-302b | UAAGUGCUUCCAUGUUUUAGUAG |
| 192 | hsa-miR-302c | AAGUGCUUCCAUGUUUCAGUGG |
| 193 | hsa-miR-302d | UAAGUGCUUCCAUGUUUGAGUGU |
| 194 | hsa-miR-3065-5p | UCAACAAAAUCACUGAUGCUGGA |
| 195 | hsa-miR-3074-5p | GUUCCUGCUGAACUGAGCCAGU |
| 196 | hsa-miR-30a | UGUAAACAUCCUCGACUGGAAGCU |
| 197 | hsa-miR-30b | UGUAAACAUCCUACACUCAGCU |
| 198 | hsa-miR-30c | UGUAAACAUCCUACACUCUCAGCU |
| 199 | hsa-miR-30d | UGUAAACAUCCCCGACUGGAAGCU |
| 200 | hsa-miR-30e | UGUAAACAUCCUUGACUGGAAGCU |
| 201 | hsa-miR-31-3p | UGCUAUGCCAACAUAUUGCCAUC |
| 202 | hsa-miR-3115 | AUAUGGGUUUACUAGUUGGU |
| 203 | hsa-miR-3117 | AUAGGACUCAUAUAGUGCCAGG |
| 204 | hsa-miR-3120 | CACAGCAAGUGUAGACAGGCA |
| 205 | hsa-miR-3124 | UUCGCGGGCGAAGGCAAAGUC |
| 206 | hsa-miR-3126-5p | UGAGGGACAGAUGCCAGAAGCA |
| 207 | hsa-miR-3127-5p | AUCAGGGCUUGUGGAAUGGGAAG |
| 208 | hsa-miR-3129-3p | AAACUAAUCUCUACACUGCUGC |
| 209 | hsa-miR-3130-3p | GCUGCACCGGAGACUGGGUAA |
| 210 | hsa-miR-3136 | CUGACUGAAUAGGUAGGGUCAU |
| 211 | hsa-miR-3138-3p | ACAGUGAGGUAGAGGGAGUGC |
| 212 | hsa-miR-3139 | UAGGAGCUCAACAGAUGCCUGUU |
| 213 | hsa-miR-3140-3p | AGCUUUUGGGAAUUCAGGUAG |
| 214 | hsa-miR-3143-5p | AUAACAUUGUAAAGCGCUUCUU |
| 215 | hsa-miR-3144 | AUAUACCUGUUCGGUCUCUUU |
| 216 | hsa-miR-3145-5p | AACUCCAAACACUCAAAACUCA |
| 217 | hsa-miR-3146-3p | CAUGCUAGGAUAGAAAGAAUGGG |
| 218 | hsa-miR-3149 | UUUGUAUGGAUAUGUGUGUGUAU |
| 219 | hsa-miR-3150-5p | CAACCUCGACGAUCUCCUCAGC |
| 220 | hsa-miR-3151 | GGUGGGGCAAUGGGAUCAGGU |
| 221 | hsa-miR-3152 | AUUGCCUCUGUUCUAACACAAG |
| 222 | hsa-miR-3155-5p | CCUCCCACUGCAGAGCCUGGGG |
| 223 | hsa-miR-3157-5p | UUCAGCCAGGCUAGUGCAGUCU |
| 224 | hsa-miR-3158 | AAGGGCUUCCUCUCUGCAGGAC |
| 225 | hsa-miR-3170 | CUGGGGUUCUGAGACAGACAGU |
| 226 | hsa-miR-3171-3p | UAUAUAGAUUCCAUAAAUCUAU |
| 227 | hsa-miR-3173 | UGCCCUGCCUGUUUUCUCCUUU |
| 228 | hsa-miR-3174 | UAGUGAGUUAGAGAUGCAGAGC |
| 229 | hsa-miR-3175 | CGGGGAGAGAACGCAGUGACGU |
| 230 | hsa-miR-3176 | ACUGGCCUGGGACUACCGGGG |
| 231 | hsa-miR-3177 | UGCACGGCACUGGGGACACGU |
| 232 | hsa-miR-3183 | GCCUCUCUCGGAGUCGCUCGGA |
| 233 | hsa-miR-3187 | UUGGCCAUGGGGCUGCGCGG |
| 234 | hsa-miR-3189 | CCCUUGGGUCUGAUGGGGUAGC |
| 235 | hsa-miR-3193 | UCCUGCGUAGGAUCUGAGGAGU |
| 236 | hsa-miR-3194-5p | GGCCAGCCACCAGGAGGGCUGC |
| 237 | hsa-miR-3198 | GUGGAGUCCUGGGGAAUGGAGA |
| 238 | hsa-miR-32 | UAUUGCACAUUACUAAGUUGC |
| 239 | hsa-miR-320-RNASEN | AAAAGCUGGGUUGAGAGGGCGA |
| 240 | hsa-miR-3200 | CACCUUGCGCUACUCAGGUCUGC |
| 241 | hsa-miR-323a | GCACAUUACACGGUCGACCUCU |
| 242 | hsa-miR-323b | CCCAAUACACGGUCGACCUCU |
| 243 | hsa-miR-324 | CGCAUCCCCUAGGGCAUUGGUGU |
| 244 | hsa-miR-325 | UUUAUUGAGGACCUCCUAUCAA |
| 245 | hsa-miR-326 | CCUCUGGGCCCUUCCUCCAG |
| 246 | hsa-miR-328 | CUGGCCCUCUCUGCCCUUCCGU |
| 247 | hsa-miR-329 | AACACACCUGGUUAACCUCUUU |
| 248 | hsa-miR-330-3p | GCAAAGCACACGGCCUGCAGAGA |
| 249 | hsa-miR-331 | GCCCCUGGGCCUAUCCUAGA |
| 250 | hsa-miR-335-5p | UCAAGAGCAAUAACGAAAAAUG |
| 251 | hsa-miR-337 | UCCUAUAUGAUGCCUUUCUUC |
| 252 | hsa-miR-338-3p | UCCAGCAUCAGUGAUUUUGUU |
| 253 | hsa-miR-339-5p | UCCCUGUCCUCCAGGAGCUCACG |
| 254 | hsa-miR-33a | GUGCAUUGUAGUUGCAUUGC |
| 255 | hsa-miR-33b | GUGCAUUGCUGUUGCAUUGC |
| 256 | hsa-miR-340 | UUAUAAAGCAAUGAGACUGAUU |
| 257 | hsa-miR-342 | UCUCACACAGAAAUCGCACCCGU |
| 258 | hsa-miR-345 | GCUGACUCCUAGUCCAGGGCU |
| 259 | hsa-miR-346 | UGUCUGCCCGCAUGCCUGCCUCU |
| 260 | hsa-miR-34a | UGGCAGUGUCUUAGCUGGUUGU |
| 261 | hsa-miR-34b | AGGCAGUGUCAUUAGCUGAUUGU |
| 262 | hsa-miR-34c | AGGCAGUGUAGUUAGCUGAUUGC |
| 263 | hsa-miR-3605-3p | CCUCCGUGUUACCUGUCCUCU |
| 264 | hsa-miR-361-5p | UUAUCAGAAUCUCCAGGGGUAC |
| 265 | hsa-miR-3611 | UUGUGAAGAAAGAAAUUCUU |
| 266 | hsa-miR-3612 | AGGAGGCAUCUUGAGAAAUGGA |
| 267 | hsa-miR-3613 | UGUUGUACUUGUUC |
| 268 | hsa-miR-3614-3p | UAGCCUUCAGAUCUUGGUGUUU |
| 269 | hsa-miR-3617 | AAAGACAUAGUUGCAAGAUGGG |
| 270 | hsa-miR-3619-5p | UCAGCAGGCAGGCUGGUGCAG |
| 271 | hsa-miR-362-5p | AAUCCUUGGAACCUAGGUGUGAGU |
| 272 | hsa-miR-3622-5p | CAGGCACGGGAGCUCAGGUGAG |
| 273 | hsa-miR-363 | AAUUGCACGGUAUCCAUCUGUA |
| 274 | hsa-miR-365 | UAAUGCCCCUAAAAAUCCUUAU |
| 275 | hsa-miR-3657 | UUGUGUCCCAUUAUUGGUGAUU |
| 276 | hsa-miR-3659 | UGAGUGUUGUCUACGAGGGCAU |
| 277 | hsa-miR-3664-5p | AACUCUGUCUUCACUCAUGAGU |
| 278 | hsa-miR-3667-3p | ACCUUCCUCUCCAUGGGUCUUU |
| 279 | hsa-miR-367 | AAUUGCACUUUAGCAAUGGUGA |
| 280 | hsa-miR-3677-3p | CUCGUGGGCUCUGGCCACGGCC |
| 281 | hsa-miR-3679-5p | UGAGGAUAUGGCAGGGAAG |
| 282 | hsa-miR-3680 | ACUCACUCACAGGAUUGUGCA |
| 283 | hsa-miR-3681 | UAGUGGAUGAUGCACUCUGUGC |
| 284 | hsa-miR-3682-3p | UGAUGAUACAGGUGGAGGUAG |
| 285 | hsa-miR-3688 | UAUGGAAAGACUUUGCCACUCU |
| 286 | hsa-miR-369 | AAUAAUACAUGGUUGAUCUUU |
| 287 | hsa-miR-3691 | UAGUGGAUGAUGGAGACUCGGU |
| 288 | hsa-miR-370 | GCCUGCUGGGGUGGAACCUGGU |
| 289 | hsa-miR-371 | ACUCAAACUGUGGGGGCACUU |
| 290 | hsa-miR-372 | AAAGUGCUGCGACAUUUGAGCGU |
| 291 | hsa-miR-373 | GAAGUGCUUCGAUUUUGGGGUGU |
| 292 | hsa-miR-374a | UUAUAAUACAACCUGAUAAGUG |
| 293 | hsa-miR-374b | AUAUAAUACAACCUGCUAAGUG |
| 294 | hsa-miR-375 | UUUGUUCGUUCGGCUCGCGUGA |
| 295 | hsa-miR-376a-3p | AUCAUAGAGGAAAAUCCACGU |
| 296 | hsa-miR-376b | AUCAUAGAGGAAAAUCCAUGU |
| 297 | hsa-miR-376c | AACAUAGAGGAAAUUCCACGU |
| 298 | hsa-miR-377 | AUCACACAAAGGCAACUUUUGU |
| 299 | hsa-miR-378 | ACUGGACUUGGAGUCAGAAGGC |
| 300 | hsa-miR-379 | UGGUAGACUAUGGAACGUAGG |
| 301 | hsa-miR-380-3p | UAUGUAAUAUGGUCCACAUCU |
| 302 | hsa-miR-381 | UAUACAAGGGCAAGCUCUCUGU |
| 303 | hsa-miR-382-5p | GAAGUUGUUCGUGGUGGAUUCG |
| 304 | hsa-miR-383 | AGAUCAGAAGGUGAUUGUGGCU |
| 305 | hsa-miR-384-3p | AUUCCUAGAAAUUGUUCAUAAU |
| 306 | hsa-miR-3909 | UGUCCUCUAGGGCCUGCAGUCU |
| 307 | hsa-miR-3910 | AAAAGGCAUAAAACCAAGACA |
| 308 | hsa-miR-3912 | UAACGCAUAAUAUGGACAUGU |
| 309 | hsa-miR-3919-5p | UACUGAGUCCUUUGUUCUCUAC |
| 310 | hsa-miR-3922 | UGUGGGACUUCUGGCCUUGACU |
| 311 | hsa-miR-3928 | GGAGGAACCUUGGAGCUUCGGC |
| 312 | hsa-miR-3934 | UCAGGUGUGGAAACUGAGGCAG |
| 313 | hsa-miR-3938 | AAUUCCCUUGUAGAUAACCCGG |
| 314 | hsa-miR-3939-3p | UACGCGCAGACCACAGGAUGUC |
| 315 | hsa-miR-3940 | CAGCCCGGAUCCCAGCCCACU |
| 316 | hsa-miR-3942-5p | AGCAAUACUGUUACCUGAAAU |
| 317 | hsa-miR-3944-5p | UGUGCAGCAGGCCAACCGAGA |
| 318 | hsa-miR-409-3p | GAAUGUUGCUCGGUGAACCCCU |
| 319 | hsa-miR-410 | AAUAUAACACAGAUGGCCUGU |
| 320 | hsa-miR-411 | AUAGUAGACCGUAUAGCGUACG |
| 321 | hsa-miR-412-3p | UUCACCUGGUCCACUAGCCG |
| 322 | hsa-miR-421 | AUCAACAGACAUUAAUUGGGCGC |
| 323 | hsa-miR-423-3p | AGCUCGGUCUGAGGCCCCUCAGU |
| 324 | hsa-miR-424 | CAGCAGCAAUUCAUGUUUUGA |
| 325 | hsa-miR-425 | AAUGACACGAUCACUCCCGUUGAGU |
| 326 | hsa-miR-429 | UAAUACUGUCUGGUAAAACCGU |
| 327 | hsa-miR-431-5p | UGUCUUGCAGGCCGUCAUGCA |
| 328 | hsa-miR-432 | UCUUGGAGUAGGUCAUUGGGUGG |
| 329 | hsa-miR-4326 | UGUUCCUCUGUCUCCCAGACUCU |
| 330 | hsa-miR-433 | AUCAUGAUGGGCUCCUCGGUGU |
| 331 | hsa-miR-448 | UUGCAUAUGUAGGAUGUCCCA |
| 332 | hsa-miR-449a | UGGCAGUGUAUUGUUAGCUGGU |
| 333 | hsa-miR-449b | AGGCAGUGUAUUGUUAGCUGGCU |
| 334 | hsa-miR-449c-3p | CAGUUGCUAGUUGCACUCCUCU |
| 335 | hsa-miR-450a | UUUUGCGAUGUGUUCCUAAUAU |
| 336 | hsa-miR-450b | UUUUGCAAUAUGUUCCUGAAUA |
| 337 | hsa-miR-451-DICER1 | AAACCGUUACCAUUACUGA |
| 338 | hsa-miR-452 | AACUGUUUGCAGAGGAAACUGA |
| 339 | hsa-miR-454 | UAGUGCAAUAUUGCUUAUAGGGU |
| 340 | hsa-miR-455-5p | UAUGUGCCUUUGGACUACAUCG |
| 341 | hsa-miR-466 | UGUGUUGCAUGUGUGUAUAUGU |
| 342 | hsa-miR-483-3p | CACUCCUCUCCUCCCGUCUUCU |
| 343 | hsa-miR-484*-RNASEN | CCGGGGGGGGCGGGGCCUCGCG |
| 344 | hsa-miR-485-3p | GUCAUACACGGCUCUCCUCUCU |
| 345 | hsa-miR-486 | UCCUGUACUGAGCUGCCCCGAG |
| 346 | hsa-miR-487a-3p | AAUCAUACAGGGACAUCCAGUU |
| 347 | hsa-miR-487b | AAUCGUACAGGGUCAUCCACUU |
| 348 | hsa-miR-488 | UUGAAAGGCUAUUUCUUGGUC |
| 349 | hsa-miR-489 | GUGACAUCACAUAUACGGCAGC |
| 350 | hsa-miR-490-5p | CCAUGGAUCUCCAGGUGGGU |
| 351 | hsa-miR-491 | AGUGGGGAACCCUUCCAUGAGGA |
| 352 | hsa-miR-493 | UUGUACAUGGUAGGCUUUCAUU |
| 353 | hsa-miR-494 | UGAAACAUACACGGGAAACCUCU |
| 354 | hsa-miR-495 | AAACAAACAUGGUGCACUUCUU |
| 355 | hsa-miR-496* | GGUUGUCCAUGGUGUGUUCAUU |
| 356 | hsa-miR-497 | CAGCAGCACACUGUGGUUUGU |
| 357 | hsa-miR-498-5p | UUUCAAGCCAGGGGGCGUUUUUC |
| 358 | hsa-miR-499 | UUAAGACUUGCAGUGAUGUUU |
| 359 | hsa-miR-500a | AUGCACCUGGGCAAGGAUUCUGA |
| 360 | hsa-miR-500b | UAAUCCUUGCUACCUGGGUGAGA |
| 361 | hsa-miR-501-3p | AAUGCACCCGGGCAAGGAUUCU |
| 362 | hsa-miR-502 | AAUGCACCUGGGCAAGGAUUCA |
| 363 | hsa-miR-503 | UAGCAGCGGGAACAGUUCUGCAG |
| 364 | hsa-miR-504 | GACCCUGGUCUGCACUCUAUC |
| 365 | hsa-miR-505 | CGUCAACACUUGCUGGUUUCCU |
| 366 | hsa-miR-506 | GUAAGGCACCCUUCUGAGUAGA |
| 367 | hsa-miR-508 | UGAUUGUAGCCUUUUGGAGUAGA |
| 368 | hsa-miR-509-3p | UGAUUGGUACGUCUGUGGGUAGA |
| 369 | hsa-miR-510-3p | UGAUUGAAACCUCUAAGAGUGGA |
| 370 | hsa-miR-511-5p | GUGUCUUUUGCUCUGCAGUCA |
| 371 | hsa-miR-512-3p | AAGUGCUGUCAUAGCUGAGGUC |
| 372 | hsa-miR-513a-3p | UAAAUUUCACCUUUCUGAGAAGG |
| 373 | hsa-miR-513b | UUCACAAGGAGGUGUCAUUUAU |
| 374 | hsa-miR-513c-5p | UUCUCAAGGAGGUGUCGUUUAU |
| 375 | hsa-miR-514a | AUUGACACUUCUGUGAGUAGA |
| 376 | hsa-miR-514b-5p | UUCUCAAGAGGGAGGCAAUCAU |
| 377 | hsa-miR-515-3p | GAGUGCCUUCUUUUGGAGCGUU |
| 378 | hsa-miR-516a | UUCUCGAGGAAAGAAGCACUUU |
| 379 | hsa-miR-516b-1 | AUCUGGAGGUAAGAAGCACUUUCU |
| 380 | hsa-miR-516b-2 | AUCUGGAGGUAAGAAGCACUUU |
| 381 | hsa-miR-517a | AUCGUGCAUCCCUUUAGAGUGU |
| 382 | hsa-miR-517b | AUCGUGCAUCCUUUUAGAGUGU |
| 383 | hsa-miR-518a-3p | GAAAGCGCUUCCCUUUGCUGGA |
| 384 | hsa-miR-518b | CAAAGCGCUCCCCUUUAGAGGU |
| 385 | hsa-miR-518c | CAAAGCGCUUCUCUUUAGAGUGU |
| 386 | hsa-miR-518d | CAAAGCGCUUCCCUUUGGAGCG |
| 387 | hsa-miR-518e-3p | AAAGCGCUUCCCUUCAGAGUGU |
| 388 | hsa-miR-518f | GAAAGCGCUUCUCUUUAGAGGA |
| 389 | hsa-miR-519a | AAAGUGCAUCCUUUUAGAGUGU |
| 390 | hsa-miR-519b | AAAGUGCAUCCUUUUAGAGGUU |
| 391 | hsa-miR-519c | AAAGUGCAUCUUUUUAGAGGAU |
| 392 | hsa-miR-519d | CAAAGUGCCUCCCUUUAGAGUGU |
| 393 | hsa-miR-519e-5p | UUCUCCAAAAGGGAGCACUUUC |
| 394 | hsa-miR-520a | CUCCAGAGGGAAGUACUUUCU |
| 395 | hsa-miR-520b-3p | AAAGUGCUUCCUUUUAGAGGGU |
| 396 | hsa-miR-520c | AAAGUGCUUCCUUUUAGAGGGU |
| 397 | hsa-miR-520d-3p | AAAGUGCUUCUCUUUGGUGGGU |
| 398 | hsa-miR-520e | AAAGUGCUUCCUUUUUGAGGGU |
| 399 | hsa-miR-520f | CAAGUGCUUCCUUUUAGAGGGU |
| 400 | hsa-miR-520g | ACAAAGUGCUUCCCUUUAGAGUGU |
| 401 | hsa-miR-520h | AAAGUGCUUCCCUUUAGAGUUA |
| 402 | hsa-miR-521 | AACGCACUUCCCUUUAGAGUGU |
| 403 | hsa-miR-522 | AAAAUGGUUCCCUUUAGAGUGU |
| 404 | hsa-miR-523-3p | AACGCGCUUCCCUAUAGAGGGU |
| 405 | hsa-miR-524 | CUACAAAGGGAAGCACUUUCUC |
| 406 | hsa-miR-525-5p | CUCCAGAGGGAUGCACUUUCUC |
| 407 | hsa-miR-526a-1-3p | GAAAGCGCUUCCUUUUAGAGGA |
| 408 | hsa-miR-526a-2-3p | GAACAUGCAUCCUUUCAGAGGG |
| 409 | hsa-miR-526b-5p | CUCUUGAGGGAAGCACUUUCUGU |
| 410 | hsa-miR-527-5p | CUGCAAAGGGAAGCCCUUUCU |
| 411 | hsa-miR-532-5p | CAUGCCUUGAGUGUAGGACCGU |
| 412 | hsa-miR-539 | AUCAUACAAGGACAAUUUCUUU |
| 413 | hsa-miR-541-3p | UGGUGGGCACAGAAUCUGGACU |
| 414 | hsa-miR-542 | UGUGACAGAUUGAUAACUGAAA |
| 415 | hsa-miR-543 | AAACAUUCGCGGUGCACUUCUU |
| 416 | hsa-miR-544-5p | UCUUGUUAAAAAGCAGAUUCU |
| 417 | hsa-miR-545-5p | UCAGUAAAUGUUUAUUAGAUGA |
| 418 | hsa-miR-549-5p | AGCUCAUCCAUAGUUGUCACUG |
| 419 | hsa-miR-550-3p | UGUCUUACUCCCUCAGGCACAU |
| 420 | hsa-miR-551a | GCGACCCACUCUUGGUUUCC |
| 421 | hsa-miR-551b | GCGACCCAUACUUGGUUUCAG |
| 422 | hsa-miR-552-3p | AACAGGUGACUGGUUAGACAA |
| 423 | hsa-miR-556-5p | GAUGAGCUCAUUGUAAUAUGA |
| 424 | hsa-miR-559-3p | UUUGGUGCAUAUUUACUUUAGG |
| 425 | hsa-miR-561 | AUCAAGGAUCUUAAACUUUGCC |
| 426 | hsa-miR-570-3p | CGAAAACAGCAAUUACCUUUGC |
| 427 | hsa-miR-574-3p | CACGCUCAUGCACACACCCACA |
| 428 | hsa-miR-576-5p | AUUCUAAUUUCUCCACGUCUUU |
| 429 | hsa-miR-577 | GUAGAUAAAAUAUUGGUACCUG |
| 430 | hsa-miR-579-3p | UUCAUUUGGUAUAAACCGCGAUU |
| 431 | hsa-miR-580 | UUGAGAAUGAUGAAUCAUUAGG |
| 432 | hsa-miR-581 | UCUUGUGUUCUCUAGAUCAGU |
| 433 | hsa-miR-582 | UUACAGUUGUUCAACCAGUUACU |
| 434 | hsa-miR-584 | UUAUGGUUUGCCUGGGACUGA |
| 435 | hsa-miR-585 | UGGGCGUAUCUGUAUGCUAGGG |
| 436 | hsa-miR-588 | UUGGCCACAAUGGGUUAGAAC |
| 437 | hsa-miR-589 | UGAGAACCACGUCUGCUCUGA |
| 438 | hsa-miR-590-5p | GAGCUUAUUCAUAAAAGUGCAG |
| 439 | hsa-miR-592 | UUGUGUCAAUAUGCGAUGAUGU |
| 440 | hsa-miR-597 | UGUGUCACUCGAUGACCACUGU |
| 441 | hsa-miR-598 | UACGUCAUCGUUGUCAUCGUCA |
| 442 | hsa-miR-599 | UUUGAUAAGCUGACAUGGGACA |
| 443 | hsa-miR-605-3p | AGAAGGCACUAUGAGAUUUAGA |
| 444 | hsa-miR-610 | UGAGCUAAAUGUGUGCUGGGA |
| 445 | hsa-miR-615-3p | UCCGAGCCUGGGUCUCCCUCU |
| 446 | hsa-miR-616-5p | ACUCAAAACCCUUCAGUGACUU |
| 447 | hsa-miR-618 | AAACUCUACUUGUCCUUCUGAGU |
| 448 | hsa-miR-624-5p | UAGUACCAGUACCUUGUGUUC |
| 449 | hsa-miR-625-3p | GACUAUAGAACUUUCCCCCUCA |
| 450 | hsa-miR-627-5p | GUGAGUCUCUAAGAAAAGAGGA |
| 451 | hsa-miR-628 | AUGCUGACAUAUUUACUAGAGG |
| 452 | hsa-miR-629 | UGGGUUUACGUUGGGAGAACUU |
| 453 | hsa-miR-641 | AAAGACAUAGGAUAGAGUCACCU |
| 454 | hsa-miR-642-3p | AGACACAUUUGGAGAGGGAAC |
| 455 | hsa-miR-643 | ACUUGUAUGCUAGCUCAGGUAG |
| 456 | hsa-miR-651 | UUUAGGAUAAGCUUGACUUUUG |
| 457 | hsa-miR-652 | AAUGGCGCCACUAGGGUUGUG |
| 458 | hsa-miR-653-3p | UUCACUGGAGUUUGUUUCAAU |
| 459 | hsa-miR-654 | UAUGUCUGCUGACCAUCACC |
| 460 | hsa-miR-655 | AUAAUACAUGGUUAACCUCUUU |
| 461 | hsa-miR-656 | AAUAUUAUACAGUCAACCUCU |
| 462 | hsa-miR-659 | AGGACCUUCCCUGAACCAAGGA |
| 463 | hsa-miR-660 | UACCCAUUGCAUAUCGGAGUUGU |
| 464 | hsa-miR-665 | ACCAGGAGGCUGAGGCCCCUCA |
| 465 | hsa-miR-668 | UGUCACUCGGCUCGGCCCACU |
| 466 | hsa-miR-670 | UUUCCUCAUAUUCAUUCAGGAGU |
| 467 | hsa-miR-671 | AGGAAGCCCUGGAGGGGCUGGAGG |
| 468 | hsa-miR-675 | CUGUAUGCCCUCACCGCUCAGC |
| 469 | hsa-miR-676 | CUGUCCUAAGGUUGUUGAGUU |
| 470 | hsa-miR-7 | UGGAAGACUAGUGAUUUUGUUGUU |
| 471 | hsa-miR-708 | AAGGAGCUUACAAUCUAGCUGG |
| 472 | hsa-miR-744 | UGCGGGGCUAGGGCUAACAGCA |
| 473 | hsa-miR-758 | UUUGUGACCUGGUCCACUAAC |
| 474 | hsa-miR-760 | CGGCUCUGGGUCUGUGGGGA |
| 475 | hsa-miR-766 | ACUCCAGCCCCACAGCCUCAGC |
| 476 | hsa-miR-767 | UGCACCAUGGUUGUCUGAGCAUGC |
| 477 | hsa-miR-769 | UGAGACCUCUGGGUUCUGAGCU |
| 478 | hsa-miR-770-5p | UCCAGUACCACGUGUCAGGGC |
| 479 | hsa-miR-873 | GCAGGAACUUGUGAGUCUCCU |
| 480 | hsa-miR-874 | CUGCCCUGGCCCGAGGGACCGA |
| 481 | hsa-miR-875-3p | CCUGGAAACACUGAGGUUGUG |
| 482 | hsa-miR-876-5p | UGGAUUUCUUUGUGAAUCACC |
| 483 | hsa-miR-885 | UCCAUUACACUACCCUGCCUCU |
| 484 | hsa-miR-887 | GUGAACGGGCGCCAUCCCGAGGCU |
| 485 | hsa-miR-888 | UACUCAAAAAGCUGUCAGUCA |
| 486 | hsa-miR-889 | UUAAUAUCGGACAACCAUUGU |
| 487 | hsa-miR-890-5p | UACUUGGAAAGGCAUCAGUUG |
| 488 | hsa-miR-891a | UGCAACGAACCUGAGCCACUGA |
| 489 | hsa-miR-891b | UGCAACUUACCUGAGUCAUUGA |
| 490 | hsa-miR-892a | CACUGUGUCCUUUCUGCGUAGA |
| 491 | hsa-miR-892b | CACUGGCUCCUUUCUGGGUAGA |
| 492 | hsa-miR-9 | UCUUUGGUUAUCUAGCUGUAUGA |
| 493 | hsa-miR-92a | UAUUGCACUUGUCCCGGCCUGU |
| 494 | hsa-miR-92b | UAUUGCACUCGUCCCGGCCUCC |
| 495 | hsa-miR-93 | CAAAGUGCUGUUCGUGCAGGUAG |
| 496 | hsa-miR-934 | UGUCUACUACUGGAGACACUGG |
| 497 | hsa-miR-937 | AUCCGCGCUCUGACUCUCUGC |
| 498 | hsa-miR-942 | UUCUCUGUUUUGGCCAUGUGU |
| 499 | hsa-miR-944 | AAAUUAUUGUACAUCGGAUGAG |
| 500 | hsa-miR-95 | UUCAACGGGUAUUUAUUGAGC |
| 501 | hsa-miR-96 | UUUGGCACUAGCACAUUUUUGCU |
| 502 | hsa-miR-98 | UGAGGUAGUAAGUUGUAUUGUU |
| 503 | hsa-miR-99a | AACCCGUAGAUCCGAUCUUGU |
| 504 | hsa-miR-99b | CACCCGUAGAACCGACCUUGCG |

miRNA

The present application measures the level of at least one miRNA in a biological sample. Samples can include any biological sample from which miRNA can be isolated. Such samples can include, but are not limited to, serum, plasma, blood, whole blood and derivatives thereof, cardiac tissue, bone marrow, urine, cerebrospinal fluid (CSF), myocardium, endothelium, skin, hair, hair follicles, saliva, oral mucous, vaginal mucous, sweat, tears, epithelial tissues, semen, seminal plasma, prostatic fluid, excreta, ascites, lymph, as well as other samples or biopsies. In one embodiment, the biological sample is plasma or serum. In other embodiments, the biological sample is cardiac tissue. The miRNA may include an intron-embedded miRNA. The miRNA may be expressed in heart tissue. The miRNA may be expressed in muscles.

In particular embodiments, the miRNA is selected from the miRNAs listed in Table 1, or listed in any of Tables 3-7. In certain embodiments, the level of each microRNA in a panel of microRNAs selected from Table 1, or from any of Tables 3-7, is measured. For instance, in another embodiment of the method, 2 or more, 3 or more, 4 or more, 5 or more, 10 or more, 15 or more, 20 or more, 25 or more, 30 or more, 35 or more, 40 or more, 50 or more, 60 or more, 70 or more, 80 or more, or 90 or more microRNAs selected from Table 1, or from any of Tables 3-7, are measured. In some embodiments, a panel of less than 20, less than 15, less than 10, or less than 5 miRNAs is tested, the panel including 1, 2, 3, 4, 5, 6, 7, 8, 9, 10, 11, 12, 13, 14, 15, 16, 17, 18, 19, 20 or more miRNAs from Table 1, or from any of Tables 3-7. The patient may be suspected of having heart failure, suspected of being in need of therapy, or is undergoing therapy for heart failure.

In another embodiment, the miRNAs detected include miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126 and miR-203 or any combination thereof. In yet another embodiment, the miRNAs detected include miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320 or any combination thereof.

The present application may also measure the level of 2, 3, 4, 5, 6 or more myomirs. As used herein, the term “myomir” may refer to any miRNA highly-enriched in cardiac and/or skeletal muscle. Myomirs may include, but are not limited to, miR-208a, miR-208b, miR-499, miR-1, miR-206, miR-133a, miR-133b, and miR-486 (McCarthy et al., 2007, MicroRNA-1 and microRNA-133a expression are decreased during skeletal muscle hypertrophy. J Appl Physiol 102, 306-313; Callis et al. 2008, Exp Biol. Med (Maywood) 233, 131-138; van Rooij et al. 2008, Trends Genet 24, 159-166; van Rooij et al. 2009 Dev Cell 17, 662-673; Small et al. 2010, Proc Natl Acad Sci. 107, 4218-4223).

The level, amount, abundance or concentration of miRNAs may be measured. The measurement result may be an absolute value or may be relative (e.g., relative to a reference oligonucleotide, relative to a reference miRNA, etc.)

Measuring or detecting the amount or level of microRNA in a sample can be performed in any manner known to one skilled in the art and such techniques for measuring or detecting the level of an miRNA are well known and can be readily employed. A variety of methods for detecting miRNAs have been described and may include small RNA sequencing (sRNAseq), deep-sequencing, single-molecule direct RNA sequencing (RNAseq), Northern blotting, microarrays, real-time PCR, RT-PCR, targeted RT-PCR, in situ hybridization, miRNA Taqman array cards, electrochemical methods (e.g., oxidation of miRNA-ligated nanoparticles), bioluminescent methods, bioluminescent protein reassembly, BRET (bioluminescence resonance energy transfer)-based methods, fluorescence correlation spectroscopy and surface-enhanced Raman spectroscopy (Cissell, K. A. and Deo, S. K. (2009) Anal. Bioanal. Chem., 394:1109-1116).

The methods of the present invention may include the step of reverse transcribing RNA when assaying the level or amount of a miRNA.

There are also commercially available kits, such as the qRT-PCR miRNA Detection Kit available from Ambion, U.S.A., which can be used for detecting and quantifying microRNA using quantitative reverse transcriptase polymerase chain reaction. TaqMan MicroRNA Assays, which employ a target-specific stern-loop reverse transcription primer to compensate for the short length of the mature miRNA, is also available from Applied Biosystems (Life Technologies, Inc., USA). qSTAR MicroRNA Detection Assays, commercially available from OriGene, Inc. (USA), can also be used. U.S. Patent Publication No. 20140024700.

Other commercially available kits, such as PAXgene Blood miRNA Kit (which uses silica-based RNA purification technology) can be employed for isolating miRNAs of 18 nucleotides or longer, available from Qiagen, USA. The miScript PCR System, a three-component system which converts miRNA and mRNA into cDNA and allows for detection of miRNAs using SYBR Green-based real-time PCR, can be employed for quantification of mature miRNA, precursor miRNA, and mRNA all from a single sample (also available from Qiagen, USA). GeneCopoeia has a commercial kit available that is based on using RT-PCR in conjunction with SYBR Green for quantitation of miRNA (All-in-One™ miRNA qRT-PCR Detection Kit, available from GeneCopoeia, Inc., USA). mirVANA, available from Life Technologies, Inc. (USA), employs glass fiber filter (GFF)-based method for isolating small RNAs.

The methods for detecting miRNAs can also include hybridization-based technology platforms and massively parallel next generation small RNA sequencing that allow for detection of multiple microRNAs simultaneously. One commercially-available hybridization-based technology utilizes a sandwich hybridization assay with signal amplification provided by a labeled branched DNA (Panornics). Another hybridization-based technology is available from Nanostring Technology (nCounter miRNA Expression Assay), where multiple miRNA sequences are detected and distinguished with fluorescently-labeled sequence tags. Examples of next-generation sequencing are available from Life Technologies (SOLiD platform) and Illumina, Inc. (e.g., Illumina HumanHT-12 bead arrays).

In one embodiment, to assay miRNA levels, the reads corresponding to miRNA genes organized in miRNA cistrons may be combined. The cistrons are labeled with the corresponding miRNA name but with the “R” of “miR” in lowercase, i.e., “mir”.

The level or amount of microRNA in a patient sample can be compared to a reference level or amount of the microRNA present in a control sample. The control sample may be from a patient or patients with a cardiovascular disease (e.g., heart failure) or a healthy subject or subjects. In other embodiments, a control sample is taken from a patient prior to treatment with a therapeutic intervention or a sample taken from an untreated patient. Reference levels for a microRNA can be determined by determining the level of a microRNA in a sufficiently large number of samples obtained from normal, healthy control subjects to obtain a pre-determined reference or threshold value. A reference level can also be determined by determining the level of the microRNA in a sample from a patient prior to treatment with the therapeutic intervention. Reference (or calibrator) level information and methods for determining reference levels can be obtained from publically available databases, as well as other sources. (See, e.g., Bunk, D. M. (2007) Clin. Biochem. Rev., 28(4):131-137; and Remington: The Science and Practice of Pharmacy, Twenty First Edition (2005)). In some embodiments, a known quantity of an oligonucleotide or oligonucleotides (e.g., small synthetic oligonucleotides with 18-25 nucleotides; or another miRNA) that is not normally present in the sample is added to the sample (i.e., the sample is spiked with a known quantity of calibrators or exogenous oligonucleotides) and the level of one or more miRNAs of interest is calculated based on the known quantity of the spiked calibrators or oligonucleotides. In one embodiment, these spike-in calibrators have no match in the human genome and serve for quantification. In another embodiment, the abundance, level or amount of the miRNA of interest is calculated from the read ratios of the miRNA reads to spiked-in calibrator reads.

The comparison of the measured levels of the one or more miRNAs to a reference amount or the level of one or more of the miRNAs in a control sample can be done by any method known to a skilled artisan. For example, comparing the amount of the microRNA in a sample to a standard amount can include comparing the ratio between 5S rRNA (or the spiked oligonucleotides) and the miRNA in a sample to a published or known ratio between 5S rRNA (or the spiked oligonucleotides) and the miRNA in a control sample.

MiRNAs can be isolated by methods described in the art for isolating small RNA molecules (U.S. Patent Publication No. 20100291580, U.S. Patent Publication No. 20100222564, U.S. Patent Publication No. 20060019258, U.S. Patent Publication No. 20110054009 and U.S. Patent Publication No. 20090023149).

In one embodiment, miRNA may be isolated from a sample by a method comprising the following steps: a) obtaining a sample having an miRNA; b) isolating total RNA from the sample; c) size fractionation of total RNA by, for example, gel electrophoresis (e.g., polyacrylamide gel electrophoresis) to separate RNAs of the appropriate sizes (e.g., small RNAs); d) ligating DNA adapters to one end or both ends of the separated small RNAs; e) reverse transcription of the adapter-ligated RNAs into cDNAs and PCR amplication; and (f) DNA sequencing. Steps (a)-(f) may be conducted in a different order than listed above. Any of the steps (a)-(f) may be skipped or combined.

Other methods for isolation of miRNA from a sample include employing a method comprising the following steps: a) obtaining a sample having an miRNA; b) adding an extraction solution to the sample; c) adding an alcohol solution to the extracted sample; d) applying the sample to a mineral or polymer support; and, e) eluting the RNA containing the miRNA from the mineral or polymer support with an ionic solution. Other procedures for isolating miRNA molecules from a sample can involve: a) adding an alcohol solution to the sample; b) applying the sample to a mineral or polymer solid support; c) eluting miRNA molecules from the support with an ionic solution; and, d) using or characterizing the miRNA molecules. (U.S. Patent Publication No. 20100222564).

MiRNA can also be isolated by methods involving separation of miRNA from mRNA, such as those described in U.S. Patent Publication No. 20060019258. These methods comprise the steps of a) providing a biological isolate including mRNA having a 5′ cap structure and small RNA having a 5′ phosphate; b) contacting the isolate with a phosphate reactive reagent having a label moiety under conditions wherein the label moiety is preferentially added to the 5′ phosphate over the 5′ cap structure, thereby producing labeled small RNA; and c) distinguishing the small RNA from the mRNA according to the presence of the label.

Examples of methods of isolating and/or quantifying microRNAs can also include but are not limited to hybridizing at least a portion of the microRNA with a fluorescent nucleic acid (a fluorescent probe), and reacting the hybridized microRNA with a fluorescent reagent, wherein the hybridized microRNA emits a fluorescent light or hybridizing at least a portion the microRNA to a radio-labeled complementary nucleic acid. There are commercially available products for fluorescent labeling and detection of miRNAs. NCode miRNA Rapid Labeling System and NCode Rapid Alexa Fluor 3 miRNA Labeling System are both commercially available from Life Technologies, Inc. (USA). Furthermore, fluorescent labels are commercially available and can include the Alexa Flour dyes (Molecular Probes), available from Life Technologies, Inc. (USA), Cy dyes (Lumiprobes), the DyLight fluorophores (available from ThermoScientific (USA)), and FluoProbes.

Locked nucleic acid probes can also be employed. For example, the miRCURY LNA microRNA ISH Optimization Kits (FFPE) provides for detection of microRNAs. This kit employs double DIG*-labeled miRCURY LNA™ microRNA Detection that can be used for in situ hybridization and is commercially available from Exiqon (USA and Denmark).

In one embodiment, a probe for detecting a miRNA can include a single-stranded molecule, including a single-stranded deoxyribonucleic acid molecule, a single-stranded ribonucleic acid molecule, a single-stranded peptide nucleic acid (PNA), or a single-stranded locked nucleic acid (LNA). The probe may be substantially complementary, for example 80%, 85%, 90%, 95%, 96%, 97%, 98%, 99% or 100% identical to the complement of the miRNA being detected, such that the probe is capable of detecting the miRNA. In some embodiments, the probe is substantially identical, 80%, 85%, 90%, 95%, 96%, 97%, 98%, 99%, or 100% identical to the miRNA, such that the probe is capable of detecting the complement of the miRNA. In some instances the probe is at least 5 nucleotides, at least 10 nucleotides, at least 15 nucleotides, at least 20 nucleotides, at least 25 nucleotides, at least 30 nucleotides or at least 40 nucleotides. In some cases, the probe may be no longer than 25 nucleotides, no longer than 35 nucleotides; no longer than 50 nucleotides; no longer than 75 nucleotides, no longer than 100 nucleotides or no longer than 125 nucleotides in length. In some embodiments the probe is substantially complementary to or substantially identical to at least 5 consecutive nucleotides of the miRNA, for example at least 6, 7, 8, 9, 10, 11, 12, 13, 14, 15, 16, 17, 18, 20, 21 and 22, or more consecutive nucleotides. In some embodiments, the probe can be 5-20, 5-25, 5-50, 50-100, or over 100 consecutive nucleotides long.

In one embodiment, a difference (increase or decrease) in the measured level of the miRNA relative to the level of the miRNA in the control sample (e.g., sample in patient prior to treatment, at a different time point during treatment, or an untreated patient) or a pre-determined reference value is indicative of the therapeutic efficacy of the therapeutic intervention. In another embodiment, an increase (or decrease) in the measured level of the miRNA relative to the level of the miRNA in the control sample or pre-determined reference value is indicative of the therapeutic efficacy of the therapeutic intervention. For instance, in such embodiments, when the level of one or more miRNAs selected from Table 1, or from any of Tables 3-7, is increased (or decreased) when compared to the level in a control sample or pre-determined reference value in response to a therapeutic intervention, the increase (or decrease) is indicative of therapeutic efficacy of the therapeutic intervention. In certain embodiments, when the level of one or more miRNAs selected from miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126 and miR-203 is increased (or decreased) when compared to the level in a control sample or pre-determined reference value in response to a therapeutic intervention, the increase (or decrease) is indicative of therapeutic efficacy of the therapeutic intervention. In another embodiment, when the level of one or more miRNAs selected from miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320 is increased (or decreased) when compared to the level in a control sample or pre-determined reference value in response to a therapeutic intervention, the increase (or decrease) is indicative of therapeutic efficacy of the therapeutic intervention.

A reduction or decrease in the measured level of the miRNA relative to the level of the miRNA in the control sample (e.g., sample in patient prior to treatment or an untreated patient) or pre-determined reference value can be indicative of the therapeutic efficacy of the therapeutic intervention. For instance, in such embodiments, when the level of one or more miRNAs selected from Table 1, or from any of Tables 3-7, is decreased (or increased) when compared to the level in a control sample or pre-determined reference value in response to a therapeutic intervention, the decrease (or increase) is indicative of therapeutic efficacy of the therapeutic intervention. In certain embodiments, when the level of one or more miRNAs selected from a group including, miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126 and miR-203 is decreased (or increased) when compared to the level in a control sample or pre-determined reference value in response to a therapeutic intervention, the decrease (or increase) is indicative of therapeutic efficacy of the therapeutic intervention. In another embodiment, when the level of one or more miRNAs selected from a group including, miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320 is decreased (or increased) when compared to the level in a control sample or pre-determined reference value in response to a therapeutic intervention, the decrease (or increase) is indicative of therapeutic efficacy of the therapeutic intervention.

Patients showing different (elevated or reduced) levels of miRNA, e.g., miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126 miR-203, miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, miR-320 or combinations (mixtures) can be identified. The expression profile of these miRNAs may be used to calculate a score for the combined or individual miRNA expression. The scores of these patients will be compared to the score of unaffected individuals. The clinical condition of these patients with respect to their cardiac status may be correlated with the miRNA expression profiles. The scores may be used to identify groups of heart failure patients responsive to treatment for heart failure.

### Cardiovascular Diseases

The methods of the present invention may be used to identify patients at risk for cardiovascular disorders or cardiovascular diseases, to evaluate a cardiovascular disease in a patient, to monitor a cardiovascular disease in a patient, or to assess efficacy of a therapy for a cardiovascular disease. Cardiovascular disorders or cardiovascular diseases can include any disorders that affect the cardiovascular system, including the heart and/or blood vessels, such as arteries and veins. Cardiovascular diseases can also include disorders affecting the kidneys. Non-limiting examples of cardiovascular diseases include heart failure, myocardial infarction, myocardial ischemia, cardiac hypertrophy, coronary heart disease, cardiac fibrosis, cardiomyopathy, ischemic heart disease, hypertensive heart disease, inflammatory heart disease, valvular heart disease, diseases of the cardiac valves, atherosclerosis, cardiorenal disease, vascular damage, myocardial damage, cardiac valvular disease or other cardiac electrophysiologic abnormalities, hypertension, or other cardiac dysfunction. Cardiovascular disease can include, but is not limited to, right-sided, left-sided failure or congestive heart failure and could be due to any one of a number of different causes. Any type of cardiovascular disease which includes impaired functioning of either the left or right ventricle is also encompassed herein. In some embodiments, cardiovascular diseases include diabetes mellitus, hyperhomocysteinemia and hypercholesterolemia.

Cardiomyopathies can include, but are not limited to, alcoholic cardiomyopathy, coronary artery disease, congenital heart disease, ischemic cardiomyopathy (ICM), dilated cardiomyopathy (DCM), hypertensive cardiomyopathy, valvular cardiomyopathy, inflammatory cardiomyopathy and myocardiodystrophy, as well as other forms of cardiomyopathies.

Hypertensive heart diseases can include, but are not limited to, left ventricular hypertrophy, coronary heart disease, heart failure (including congestive), hypertensive cardiomyopathy, cardiac arrhythmias and renal disorders.

Inflammatory heart diseases can include, but are not limited to, endocarditis, inflammatory cardiomegaly and myocarditis.

Heart failure may be classified according to the severity of the symptoms. Table 2 describes the most commonly used classification system, the New York Heart Association (NYHA) Functional Classification. It places patients in one of four categories based on how much they are limited during physical activity.

TABLE 2
NYHA Functional Classification of Heart Failure

| Class | Patient Symptoms |
|---|---|
| Class I | No limitation of physical activity. Ordinary physical |
| (Mild) | activity does not cause undue fatigue, palpitation, |
| | or dyspnea (shortness of breath). |
| Class II | Slight limitation of physical activity. Comfortable at |
| (Mild) | rest, but ordinary physical activity results in fatigue, |
| | palpitation, or dyspnea. |
| Class III | Marked limitation of physical activity. Comfortable at |
| (Moderate) | rest, but less than ordinary activity causes fatigue, |
| | palpitation, or dyspnea. |
| Class IV | Unable to carry out any physical activity without dis- |
| (Severe) | comfort. Symptoms of cardiac insufficiency at rest. If any |
| | physical activity is undertaken, discomfort is increased. |

The methods of the present invention may also be used to establish risk profiles for developing heart failure.

The analysis of risk profiles or staging classification may be done using logistic regression together with a variety of threshold classifiers. U.S. Patent Publication No. 20120153744.

### Samples

Sampling methods are well known by those skilled in the art and any applicable techniques for obtaining biological samples of any type are contemplated and can be employed with the methods of the present invention. (See, e.g., Clinical Proteomics: Methods and Protocols, Vol. 428 in Methods in Molecular Biology, Ed. Antonia Vlahou (2008).)

The samples may be drawn before, during or after therapy. The samples may be drawn at different time points during therapy, and/or be drawn at different time points after therapy. It will be appreciated that one of ordinary skill in the art such as a physician can determine when to draw samples.

When the sample is drawn during the therapeutic intervention, it can be obtained from the subject at any point following the initiation of the therapeutic intervention. In some embodiments, the sample is obtained about 1 week, about 2 weeks, about 3 weeks, about 1 month, about 2 months, about 3 months, about 4 months, about 5 months, about 6 months, at least 1, 2, 3, or 6 months following the start of the therapeutic intervention. In some embodiments, the sample is obtained least 1, 2, 3, 4, 6 or 8 weeks following the start of the therapeutic intervention. In some embodiments, the sample is obtained at least 1, 2, 3, 4, 5, 6, or 7 days following the start of the therapeutic intervention. In some embodiments, the sample is obtained at least 1 hour, 6 hours, 12 hours, 18 hours or 24 hours after the start of the therapeutic intervention. In other embodiments, the sample is obtained at least one week following the start of the therapeutic intervention. In some embodiments, one or more miRNAs selected from Table 1, or selected from Tables 3-7, is measured between 1 and 8 weeks, between 2 and 7 weeks, at 1, 2, 3, 4, 5, 6, 7 or 8 weeks following therapy.

### Therapeutic Intervention

The present invention provides for methods for evaluating and/or monitoring the efficacy of a therapeutic intervention for treating a cardiovascular disease. These methods can include the step of measuring the level of at least one miRNA, such as one or more miRNAs listed in Table 1, or listed in any of Tables 3-7, or a panel of miRNAs, in a biological sample from a patient receiving a therapeutic intervention. In some embodiments, the level of the at least one miRNA in the biological sample is compared to a reference level, or the level of the at least one miRNA in a control sample. The measured level of the at least one miRNA is indicative of the therapeutic efficacy of the therapeutic intervention. In some cases, an increase or decrease in the level of the miRNA is indicative of the efficacy of the therapeutic intervention. In some embodiments, a change in the measured level of the at least one miRNA relative to a sample from the patient taken prior to treatment or earlier during the treatment regimen is indicative of the therapeutic efficacy of the therapeutic intervention.

In certain embodiments, the method comprises detecting 1, 2, 3, 4, 5, 6, 7, 8, 9, 10, 11, 12, 13, 14, 15, 16, 17, 18, 19, 20 or more miRNAs (e.g., including all miRNAs) listed in Table 1, or listed in any of Tables 3-7. When a panel of miRNAs is determined in the patient sample, the patient sample may be classified as indicative of effective or non-effective intervention on the basis of a classifier algorithm. For example, samples may be classified on the basis of threshold values as described, or based upon mean and/or median miRNA levels in one population or versus another (e.g., a population of healthy controls and population of patients with heart failure, or levels based on effective versus ineffective therapy).

Various classification schemes are known for classifying samples between two or more classes or groups, and these include, without limitation: Principal Components Analysis, Naive Bayes, Support Vector Machines, Nearest Neighbors, Decision Trees, Logistic, Artificial Neural Networks, Penalized Logistic Regression, and Rule-based schemes. In addition, the predictions from multiple models can be combined to generate an overall prediction. Thus, a classification algorithm or “class predictor” may be constructed to classify samples. The process for preparing a suitable class predictor (reviewed in Simon (2003) British Journal of Cancer (89) 1599-1604).

The present invention also provides methods for modifying the treatment regimen of a therapeutic entity comprising detecting the level of at least one miRNA in a biological sample from a patient receiving the therapeutic intervention and modifying the treatment regimen based on an increase or decrease in the level of the at least one miRNA in said biological sample. The methods for modifying the treatment regimen of a therapeutic intervention may comprise the steps of: (a) detecting the level of at least one miRNA, such as one or more miRNAs listed in Table 1, or listed in any of Tables 3-7, in a biological sample from a patient receiving the therapeutic intervention; and (b) modifying the treatment regimen based on an increase or decrease in the level of the at least one miRNA in the biological sample. In some embodiments, the method comprises detecting 2, 3, 4, 5, 6, 7, 8, 9, 10 or more miRNAs (e.g., including all miRNAs) listed in Table 1, or listed in any of Tables 3-7. In some such embodiments, less than 100, less than 50, or less than 25 miRNAs are detected, including the miRNAs from Table 1, or listed in any of Tables 3-7.

Modifying the treatment regimen can include, but is not limited to, changing and/or modifying the type of therapeutic intervention, the dosage at which the therapeutic intervention is administered, the frequency of administration of the therapeutic intervention, the route of administration of the therapeutic intervention, as well as any other parameters that would be well known by a physician to change and/or modify. For example, where miRNAs of Table 1, or of any of Tables 3-7, decrease (or increase) during therapy or match reference levels, the therapeutic intervention is continued. In embodiments where miRNAs of Table 1, or of any of Tables 3-7, do not decrease (or increase) during therapy or match reference levels, the therapeutic intervention is modified.

In another embodiment, the information regarding the increase or decrease in the level of at least one miRNA can be used to determine the treatment efficacy of treatment with the therapeutic intervention, as well as to tailor the treatment regimens of therapeutic interventions.

In another embodiment, the treatment efficacy can be used to determine whether to continue, discontinue, or modify a therapeutic intervention. The treatment efficacy can also be used to determine whether to increase or decrease the dosage of a therapeutic intervention. In some embodiments the treatment efficacy can be used to determine whether to change the dosing frequency of a therapeutic intervention. Further, the treatment efficacy can be used to determine whether to change the number or the frequency of administration of the therapeutic intervention. In some embodiments, the treatment efficacy can be used to determine whether to change the number of doses per day, per week, times per day or can be used to determine whether to change the dosage amount.

The term “indicative of the therapeutic efficacy” can include any methods for determining that a therapeutic intervention is providing a benefit to a patient. The terms “therapeutic efficacy” are generally indicated by alleviation of one or more signs or symptoms associated with a cardiovascular disease and alleviation of one or more signs or symptoms of the cardiovascular disease being treated can be readily determined by one skilled in the art. “Therapeutic efficacy” may also refer to the prevention or amelioration of signs and symptoms of toxicities typically associated with standard therapeutic interventions for cardiovascular diseases.

Evidence of therapeutic efficacy may be specific to the cardiovascular disease being treated and can include evidence well known in the art. For example, evidence of therapeutic efficacy can include but is not limited to improvement or alleviation of one or more symptoms of cardiac hypertrophy, heart failure, or myocardial infarction in the subject, or in the delay in the transition from cardiac hypertrophy to heart failure. The one or more improved or alleviated symptoms can include, for example, increased exercise capacity, increased cardiac ejection volume, decreased left ventricular end diastolic pressure, decreased pulmonary capillary wedge pressure, increased cardiac output, increased cardiac index, lowered pulmonary artery pressures, decreased left ventricular end systolic and diastolic dimensions, decreased cardiac fibrosis, decreased collagen deposition in cardiac muscle, decreased left and right ventricular wall stress, decreased wall tension, increased quality of life, and decreased disease related morbidity or mortality. Further, therapeutic efficacy can also include general improvements in the overall health of the patient, such as but not limited to enhancement of patient life quality, increase in predicted survival rate, decrease in depression or decrease in rate of recurrence of the indication (Physicians' Desk Reference (2010).

Efficacy of a therapeutic intervention can also include evaluating or monitoring for the improvement of one or more symptoms of cardiac hypertrophy, heart failure, or myocardial infarction in the subject, or for the delay in the transition from cardiac hypertrophy to heart failure. The one or more improved symptoms may include, for example, increased exercise capacity, increased cardiac ejection volume, decreased left ventricular end diastolic pressure, decreased pulmonary capillary wedge pressure, increased cardiac output, increased cardiac index, lowered pulmonary artery pressures, decreased left ventricular end systolic and diastolic dimensions, decreased cardiac fibrosis, decreased collagen deposition in cardiac muscle, decreased left and right ventricular wall stress, decreased wall tension, increased quality of life and decreased disease related morbidity or mortality. The measured levels of plasma miRNAs may serve as a surrogate marker for efficacy of the therapeutic intervention.

Therapeutic interventions can include, pharmacologic intervention, devices, surgical intervention, or any combination thereof. Pharmacologic interventions may include, but are not limited to, treatment with diuretics, vasodilators, inotropic agents (i.e., compounds that increase cardiac contractility), ACE inhibitors, beta blockers, neurohumoral blockers (e.g., beta-blockers, angiotensin converting enzyme inhibitors), and aldosterone antagonists (e.g., spironolactone, eplerenone). Devices may include, e.g., a bi-ventricular pacemarker, implantable cardioverter-defibrillator (ICD), ventricular assist device (VAD), left ventricular assist device (LVAD), or cardiac resynchronization therapy (CRT). Surgical interventions may include, heart transplantation, artificial heart, etc.

In certain embodiments, therapeutic intervention can be implantation of a medical device or surgical, which includes, for example, preventative, diagnostic or staging, curative and palliative surgery. Surgery may be used in conjunction with other therapies, including one or more other agents as described herein. Such surgical therapeutic agents for vascular and cardiovascular diseases and disorders are well known to those of skill in the art, and may include, but are not limited to, providing a cardiovascular mechanical prostheses, angioplasty, coronary artery reperfusion, catheter ablation, providing an implantable cardioverter defibrillator to the subject, mechanical circulatory support or a combination thereof. Examples of a mechanical circulatory support that may be used in the present invention comprise an intra-aortic balloon counterpulsation, left ventricular assist device (LVAD) or combinations thereof.

Pharmacologic agents for therapeutic interventions can include, but are not limited to, miRNA based therapeutics (including antisense oligonucleotides), antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof. U.S. Patent Application No. 2010/0317713.

In various embodiments, the therapeutic intervention is a miRNA-based therapy. In some embodiments, the miRNA based therapeutic is an antisense oligonucleotide. The antisense oligonucleotides may be ribonucleotides or deoxyribonucleotides. In some embodiments, the miRNA based therapeutic is an antisense oligonucleotide targeting a miRNA expressed in heart tissue. The antisense oligonucleotide therapeutics may have at least one chemical modification (i.e., the oligonucleotide is chemically modified). For instance, suitable antisense oligonucleotides may be comprised of one or more conformationally constrained or bicyclic sugar nucleoside modifications, for example, locked nucleic acids (LNAs) in some embodiments, the miRNA based therapeutic is a chemically-modified antisense oligonucleotide. In some embodiments, the miRNA based therapeutic is a chemically-modified antisense oligonucleotide targeting a miRNA expressed in heart tissue.

Alternatively, the antisense oligonucleotides may comprise peptide nucleic acids (PNAs), which contain a peptide-based backbone rather than a sugar-phosphate backbone. Other chemical modifications that the antisense oligonucleotides may contain include, but are not limited to, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages (U.S. Pat. Nos. 6,693,187 and 7,067,641). For instance, antisense oligonucleotides, particularly those of shorter lengths (e.g., less than 15 nucleotides) can comprise one or more affinity enhancing modifications, such as, but not limited to, LNAs, bicyclic nucleosides, phosphonoformates, 2′ O alkyl and the like.

In other embodiments, suitable antisense oligonucleotides are 2′-O-methoxyethyl S gapmers which contain 2′-O-methoxyethyl-modified ribonucleotides on both 5′ and 3′ ends with at least ten deoxyribonucleotides in the center. These gapmers are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the invention. Preferable antisense oligonucleotides useful for inhibiting the activity of miRNAs are about 5 to about 50 nucleotides in length, about 10 to about 30 nucleotides in length, about 8 to about 18 nucleotides, about 12 to 16 nucleotides, about 8 nucleotides or greater, or about 20 to about 25 nucleotides in length.

In certain embodiments, antisense oligonucleotides may comprise a sequence that is at least partially complementary to a mature miRNA sequence, e.g., at least about 75%, 80%, 85%, 90%, 95%, 96%, 97%, 98%, or 99% complementary to a mature miRNA sequence. In some embodiments, the antisense oligonucleotide may be substantially complementary to a mature miRNA sequence, that is at least about 95%, 96%, 97%, 98%, or 99% complementary to a target miRNA sequence. In one embodiment, the antisense oligonucleotide comprises a sequence that is 100% complementary to a mature miRNA sequence.

Locked nucleic acids (LNAs) are modified nucleotides that contain an extra bridge between the 2′ and 4′ carbons of the ribose sugar moiety resulting in a locked conformation that confers enhanced thermal stability to oligonucleotides containing the LNAs. LNAs are described, for example, in U.S. Pat. No. 6,268,490, U.S. Pat. No. 6,316,198, U.S. Pat. No. 6,403,566, U.S. Pat. No. 6,770,748, U.S. Pat. No. 6,833,361, U.S. Pat. No. 6,998,484, U.S. Pat. No. 6,670,461, and U.S. Pat. No. 7,034,133.

In other embodiments, the antisense oligonucleotides are antagomirs. Antagomirs are single-stranded, chemically-modified ribonucleotides that are at least partially complementary to the miRNA sequence. Antagomirs may comprise one or more modified nucleotides, such as 2′-O-methyl-sugar modifications. In some embodiments, antagomirs comprise only modified nucleotides. Antagomirs may also comprise one or more phosphorothioate linkages resulting in a partial or full phosphorothioate backbone. To facilitate in vivo delivery and stability, the antagomir may be linked to a steroid such as cholesterol, a fatty acid, a vitamin, a carbohydrate, a peptide or another small molecule ligand at its 3′ end. Antagomirs suitable for inhibiting miRNAs may be about 15 to about 50 nucleotides in length, about 18 to about 30 nucleotides in length, or about 20 to about 25 nucleotides in length. “Partially complementary” refers to a sequence that is at least about 75%, 80%, 85%, 90%, 95%, 96%, 97%, 98%, or 99% complementary to a target polynucleotide sequence. The antagomirs may be at least about 75%, 80%, 85%, 90%, 95%, 96%, 97%, 98%, or 99% complementary to a mature miRNA sequence. In some embodiments, the antagomir may be substantially complementary to a mature miRNA sequence, that is at least about 95%, 96%, 97%, 98%, or 99% complementary to a target polynucleotide sequence. In other embodiments, the antagomirs are 100% complementary to the mature miRNA sequence.

In another embodiment, the therapeutic intervention is an antisense oligonucleotide targeting miR-208a and/or miR-208b, or a chemically-modified antisense oligonucleotide targeting miR-208a and/or miR-208b. In certain such embodiments, a change in the measured level of the miRNA relative to the level of the miRNA in the control sample or pre-determined reference value is indicative of decreased expression of miR-208a and/or miR-208b in heart tissue. WO 2008/016924, WO 2009/018492, WO 2010/091204, PCT/US2010/023234.

An antihyperlipoproteinemic may be an agent that lowers the concentration of one of more blood lipids and/or lipoproteins. Examples of antihyperlipoproteinemics can include but are not limited to, acifran, azacosterol, benfluorex, p-benzalbutyramide, carnitine, chondroitin sulfate, clomestrone, detaxtran, dextran sulfate sodium, 5, 8, 11, 14, 17-eicosapentaenoic acid, eritadenine, furazabol, meglutol, melinamide, mytatrienediol, ornithine, y-oryzanol, pantethine, pentaerythritol tetraacetate, alpha-phenylbutyramide, pirozadil, probucol (lorelco), p-sitosterol, sultosilic acid-piperazine salt, tiadenol, triparanol and xenbucin. In some embodiments, antihyperlipoproteinemic agents can further comprise an aryloxyalkanoicifibric acid derivative, a resin/bile acid sequesterant, an HMG CoA reductase inhibitor, a nicotinic acid derivative, a thyroid hormone or thyroid hormone analog, a miscellaneous agent or a combination thereof.

In another embodiment, administration of an agent that aids in the removal or prevention of blood clots may be combined with administration of a modulator, particularly in treatment of athersclerosis and vasculature (e.g., arterial) blockages. Examples of antithrombotic and/or fibrinolytic agents can include but are not limited to anticoagulants, anticoagulant antagonists, antiplatelet agents, thrombolytic agents, throinbolytic agent antagonists or combinations thereof. Antithrombotic agents that can be included are those that are administered orally, such as, for example, aspirin and warfarin (coumadin).

Anticoagulants can include but are not limited to acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodiuim, oxazidione, pentosan polysulfate, phenindione, phenprocoumon, phosvitin, picotamide, tioclomarol and warfarin.

Antiplatelet agents can include but are not limited to aspirin, a dextran, dipyridamole (persantin), heparin, sulfinpyranone (anturane) and ticlopidine (ticlid).

Thrombolytic agents can include but are not limited to tissue plasminogen activator (activase), plasmin, pro-urokinase, urokinase (abbokinase) streptokinase (streptase) and anistreplasel APSAC (eminase).

In one embodiment, the therapeutic intervention is an antiarrhythmic agent.

Antiarrhythmic agents can include, but are not limited to Class I antiarrhythmic agents (sodium channel blockers), Class II antiarrhythmic agents (beta-adrenergic blockers), Class III antiarrhythmic agents (repolarization prolonging drugs), Class IV antiarrhythmic agents (calcium channel blockers) and miscellaneous antiarrhythric agents. Examples of sodium channel blockers can include but are not limited to Class IA, Class IB and Class IC antiarrhythmic agents. Non-limiting examples of Class IA antiarrhythmic agents include disppyramide (norpace), procainamide (pronestyl) and quinidine (quinidex). Examples of Class IB antiarrhythmic agents can include but are not limited to lidocaine (xylocalne), tocamide (tonocard) and mexiletine (mexitil). Examples of Class IC antiarrhythmic agents can include but are not limited to encamide (enkaid) and flecamide (tambocor).

Examples of a beta blocker, otherwise known as a p-adrenergic blocker, a p-adrenergic antagonist or a Class II antiarrhythmic agent, can include but are not limited to acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In some embodiments, the beta blocker can comprise an aryloxypropanolamine derivative. Examples of aryloxypropanolamine derivatives can include but are not limited to acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, mrnoprolol, nadolol, nipradilol, oxprenolol, penbutolol, pindolol, propanolol, talinolol, tertatolol, tinolol and toliprolol.

Examples of agents that prolong repolarization, also known as a Class III antiarrhythmic agent, can include but are not limited to include amiodarone (cordarone) and sotalol (betapace).

Examples of a calcium channel blocker, otherwise known as a Class IV antiarrhythmic agent, can include but are not limited to an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexyline. In some embodiments, a calcium channel blocker comprises a long-acting dihydropyridine (nifedipine-type) calcium antagonist.

Examples of antihypertensive agents can include but are not limited to sympatholytic, alpha/beta blockers, alpha blockers, anti-angiotensin II agents, beta blockers, calcium channel blockers, vasodilators and miscellaneous antihypertensives.

Examples of an alpha blocker, also known as an α-adrenergic blocker or an α-adrenergic antagonist, can include but are not limited to, amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Quinazoline derivatives can include but are not limited to alfuzosin, bunazosin, doxazosin, prazosin, terazosin and trimazosin. The antihypertensive agent may be both an alpha and beta adrenergic antagonist. Examples of an alpha/beta blocker can include but are not limited to labetalol (normodyne, trandate).

Examples of anti-angiotensin II agents can include but are not limited to angiotensin converting enzyme inhibitors and angiotensin II receptor antagonists. Angiotensin converting enzyme inhibitors (ACE inhibitors) can include but are not limited to alacepril, enalapril (vasotec), captopril, cilazapril, delapril, enalaprilat, fosinopril, lisinopril, moveltopril, perindopril, quinapril and ramipril. Examples of an angiotensin II receptor blocker, also known as an angiotensin II receptor antagonist, an ANG receptor blocker or an ANG-II type-I receptor blocker (ARBS), include but are not limited to angiocandesartan, eprosartan, irbesartan, losartan and valsartan.

Examples of a sympatholytic include a centrally acting sympatholytic or a peripherally acting sympatholytic. Examples of a centrally acting sympatholytic, also known as an central nervous system (CNS) sympatholytic, can include but are not limited to clonidine (catapres), guanabenz (wytensin) guanfacine (tenex) and methyldopa (aldomet).

Examples of a peripherally acting sympatholytic can include but are not limited to a ganglion blocking agent, an adrenergic neuron blocking agent, .beta.-adrenergic blocking agent or an alpha1-adrenergic blocking agent. Examples of a ganglion blocking agent include mecamylamine (inversine) and trimethaphan (arfonad). Examples of an adrenergic neuron blocking agent can include but are not limited to guanethidine (ismelin) and reserpine (serpasil). Examples of a beta-adrenergic blocker can include but are not limited to acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren).

Examples of alpha1-adrenergic blocker can include but are not limited to prazosin (minipress), doxazocin (cardura) and terazosin (hytrin).

The therapeutic intervention can also comprise a vasodilator (e.g., a cerebral vasodilator, a coronary vasodilator or a peripheral vasodilator). In other embodiments, a vasodilator comprises a coronary vasodilator. Examples of a coronary vasodilator include but are not limited to amotriphene, bendazol, benfurodil hemisuccinate, benziodarone, chlioracizine, chromonar, clobenfurol, clonitrate, dilazep, dipyridamole, droprenilamine, efloxate, erythrityl tetranitrane, etafenone, fendiline, floredil, ganglefene, herestrol bis(p-dinoeylaminoethyl ether), hexobendine, itramin tosylate, khellin, lidoflanine, mannitol hexanitrane, medibazine, nicorglycerin, pentaerythritol tetranitrate, pentrinitrol, perhexyline, pimefyiline, trapidil, tricromyl, trimetazidine, troInitrate phosphate and visnadine. In some embodiments, a vasodilator can comprise a chronic therapy vasodilator or a hypertensive emergency vasodilator. Examples of a chronic therapy vasodilator can include but are not limited to hydralazine (apresoline) and minoxidil (loniten). Examples of a hypertensive emergency vasodilator can include but are not limited to nitroprusside (nipride), diazoxide (hyperstat IV), hydralazine (apresoline), minoxidil (loniten) and verapamil.

Examples of antihypertensives can also include, but are not limited to, ajmaline, gamma-amino butyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzo limine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain embodiments, an antihypertensive can comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quaternary ammoniam compound, a reserpine derivative or a suflonamide derivative. Examples of arylethanolamine derivatives can include but are not limited to amosulalol, bufuralol, dilevalol, labetalol, pronethalol, sotalol and sulfinalol. Examples of benzothiadiazine derivatives can include but are not limited to althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide. Examples of N-carboxyalkyl(peptide/lactam) derivatives can include but are not limited to alacepril, captopril, cilazapril, delapril, enalapril, enalaprilat, fosinopril, lisinopril, moveltipril, perindopril, quinapril and ramipril. Examples of dihydropyridine derivatives can include but are not limited to amlodipine, felodipine, isradipine, nicardipine, nifedipine, nilvadipine, nisoldipine and nitrendipine. Examples of guanidine derivatives can include but are not limited to bethanidine, debrisoquin, guanabenz, guanacline, guanadrel, guanazodine, guanethidine, guanfacine, guanochlor, guanoxabenz and guanoxan. Examples of hydrazines/phthalazines can include but are not limited to budralazine, cadralazine, dihydralazine, endralazine, hydracarbazine, hydralazine, pheniprazine, pildralazine and todralazine. Examples of imidazole derivatives can include but are not limited to clonidine, lofexidine, phentolamine, tiamenidine and tolonidine. Examples of quaternary ammonium compounds can include but are not limited to azamethonium bromide, chlorisondamine chloride, hexamethonium, pentacynium bis(methylsulfate), pentamethoniumi bromide, pentolinium tartrate, phenactropiniutm chloride and trimethidinium methosulfate. Examples of reserpine derivatives can include but are not limited to bietaserpine, deserpidine, rescinnamine, reserpine and syrosingopine. Examples of sulfonamide derivatives can include but are not limited to ambuside, clopamide, furosemide, indapamide, quinethazone, trip amide and xipamide.

Examples of agents for the treatment of congestive heart failure can include but are not limited to anti-angiotensin II agents, afterload-preload reduction treatment, diuretics and inotropic agents.

Examples of a diuretic can include but are not limited to a thiazide or benzothiadiazine derivative (e.g., althiazide, bendroflumethazide, benzthiazide, benzylhydrochiorchlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamnphamide, mercaptomerin sodium, mercumallylic acid, mercumatilin dodium, mercurous chloride, mersalyl), a pteridine (e.g., furtherene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenami de, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, trip amide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzo limine, perhexyline, ticmafen and urea.

Examples of a positive inotropic agent, also known as a cardiotonic, can include but are not limited to acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol. In some embodiments, an intropic agent is a cardiac glycoside, a beta-adrenergic agonist or a phosphodiesterase inhibitor. Examples of a cardiac glycoside can include but are not limited to digoxin (lanoxin) and digitoxin (crystodigin). Examples of a .beta.-adrenergic agonist include but are not limited to albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Examples of a phosphodiesterase inhibitor can include but are not limited to aminone (inocor).

Antianginal agents may comprise organonitrates, calcium channel blockers, beta blockers and combinations thereof. Examples of organonitrates, also known as nitrovasodilators, can include but are not limited to nitroglycerin (nitro-bid, nitrostat), isosorbide dinitrate (isordil, sorbitrate) and amyl nitrate (aspirol, vaporole).

Endothelin (ET) is a 21-amino acid peptide that has potent physiologic and pathophysiologic effects that appear to be involved in the development of heart failure. The effects of ET are mediated through interaction with two classes of cell surface receptors. Inhibiting the ability of ET to stimulate cells involves the use of agents that block the interaction of ET with its receptors. Examples of endothelin receptor antagonists (ERA) can include but are not limited to Bosentan, Enrasentan, Ambrisentan, Darusentan, Tezosentan, Atrasentan, Avosentan, Clazosentan, Edonentan, sitaxsentan, TBC 3711, BQ 123, and BQ 788.

### Kits

Another aspect of the invention is a kit containing a reagent or reagents for measuring at least one miRNA in a biological sample, instructions for measuring the at least one miRNA, and/or instructions for evaluating or monitoring the efficacy of a therapeutic intervention for treating a cardiovascular disease in a patient based on the level of the at least one miRNA, and/or instructions for predicting or assessing the level of severity of heart failure or heart failure progression in a patient. In some embodiments, the kit contains reagents for measuring from 2 to about 20 human miRNAs, including at least 3, 4, 5, 6, 7, 8, 9, 10, 11, 12, 13, 14, 15 or more from Table 1, or from any of Tables 3-7.

In one embodiment, the kit reagent comprises a miRNA-specific primer and/or probe for reverse transcribing, amplifying, and/or hybridizing to one or more miRNAs described herein. Such kits can further comprise one or more normalization controls and/or a TaqMan probe specific for each miRNA of the kit.

Any of the compositions described herein may be comprised in a kit. In one embodiment, the kit contains a reagent for measuring at least one miRNA selected from Table 1, or selected from any of Tables 3-7, in a biological sample, instructions for measuring the at least one miRNA and instructions for evaluating or monitoring the efficacy of a therapeutic intervention for treating a cardiovascular disease in a patient based on the level of the at least one miRNA. In some embodiments, the kit contains reagents for measuring the level of at least 2, 3, 4, 5, 6 or 10 miRNAs (or more), from Table 1, or from any of Tables 3-7. The kit may also be customized for determining the efficacy of therapy for heart failure, and thus provides the reagents for determining 50 or fewer, 40 or fewer, 30 or fewer, or 25 or fewer miRNAs, including the miRNAs of Table 1, or of any of Tables 3-7.

In another embodiment, the kit contains a reagent for measuring one or more miRNAs selected from miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126 and miR-203, instructions for measuring one or more of these miRNAs, and instructions for evaluating or monitoring the efficacy of a therapeutic intervention for treating a cardiovascular disease in a patient based on the level of one or more of these miRNAs.

In yet another embodiment, the kit contains a reagent for measuring one or more miRNAs selected from miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320, instructions for measuring one or more of these miRNAs, and instructions for evaluating or monitoring the efficacy of a therapeutic intervention for treating a cardiovascular disease in a patient based on the level of one or more of these miRNAs.

In certain embodiments, the kit can further contain one or more normalization controls. The one or more normalization controls are provided as one or more separate reagents for spiking samples or reactions. The normalization control can be added in a range of from about 0.1 fmol to about 5 mol. In some embodiments, the normalization control is added at about 0.1 fmol, 0.5 fmol, 1 fmol, 2 fmol, 3 fmol, 4 fmol or 5 fmol. In some embodiments, the at least one normalization control is a non-endogenous RNA or miRNA, or a miRNA not expressed in the sample.

The kit can further contain a TaqMan probe specific for each miRNA of the kit. In some embodiments, the TaqMan probe is specific for a miRNA selected from the group consisting of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126 and miR-203. In another embodiment, the TaqMan probe is specific for a miRNA selected from the group consisting of miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320.

The kit is contemplated for use with a biological sample from a patient receiving treatment for a cardiovascular disease. In further embodiments, the biological sample is plasma or serum obtained from a patient receiving treatment for a cardiovascular disease, such as, heart failure, myocardial infarction, pathologic cardiac hypertrophy, or hypertension. The treatment may be LVAD implantation.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed (e.g., sterile, pharmaceutically acceptable buffer and/or other diluents). However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means.

Such kits may also include components that preserve or maintain the reagents or that protect against their degradation. Such components may be DNAse-free, RNAse-free or protect against nucleases (e.g., RNAses and DNAses). Such kits generally will comprise, in suitable means, distinct containers for each individual reagent or solution.

A kit will also include instructions for employing the kit components as well the use of any other reagent not included in the kit. Instructions may include variations that can be implemented.

The invention may also encompass biochips. Biochips contain a microarray of probes which are capable of hybridizing to the miRNAs described herein. The probes may either be synthesized first, with subsequent attachment to the biochip, or may be directly synthesized on the biochip.

Compounds may be tested for their effectiveness in modulating miRNA expression in cells, transgenic animals or mammalian subjects as follows. Cells over-expressing miRNAs will be constructed using standard transfection techniques. Rooij 108: 219 (2011); Lennox et al. Pharm Res. 27:1788 (2010). The transfected cells will be contacted with various compounds and miRNA expression assayed. A variety of different compounds are known to inhibit miRNAs, including anti-miRNAs or antagomirs. Rooij (2011) Circulation Research 108: 219.

Transgenic animal models, where selected miRNAs are expressed using site and stage-specific promoters, will also be used. The ability of various compounds to modulate miRNA expression in vivo will also be tested. Id.

The following are examples of the present invention and are not to be construed as limiting.

### Example 1

### RNA-Sequencing Analysis of Myocardial and Circulating Small RNAs in Human Heart Failure

We determined myocardial and circulating miRNA abundance and its changes in patients with stable and end-stage heart failure before and at different time points after mechanical unloading by a left ventricular assist device (LVAD) by small-RNA-sequencing. MiRNA changes in failing heart tissues partially resembled that of fetal myocardium. Consistent with prototypical miRNA-target-mRNA interactions, target mRNA levels were negatively correlated to changes in abundance for highly expressed miRNAs in heart failure and fetal hearts. The circulating small RNA profile was dominated by miRNAs, tRNAs and small cytoplasmic RNAs. Heart- and muscle-specific circulating miRNAs (myomirs) increased up to 140-fold in advanced heart failure, which coincided with a similar increase in cardiac troponin I protein, the established marker for heart injury. These extracellular changes nearly completely reversed 3 months following initiation of LVAD support. In stable heart failure, circulating miRNAs showed less than 5-fold differences compared to normal, and myomir and cardiac troponin I levels were only captured near the detection limit. These findings emphasize the usefulness of circulating miRNAs as biomarkers for heart injury.

In heart tissues, we observed that changes in abundant miRNAs coincided with seed-dependent mRNA target responses indicative of active miRNA regulation during development and disease.

(a) miRNA Profiles in the Left-Ventricular Myocardium

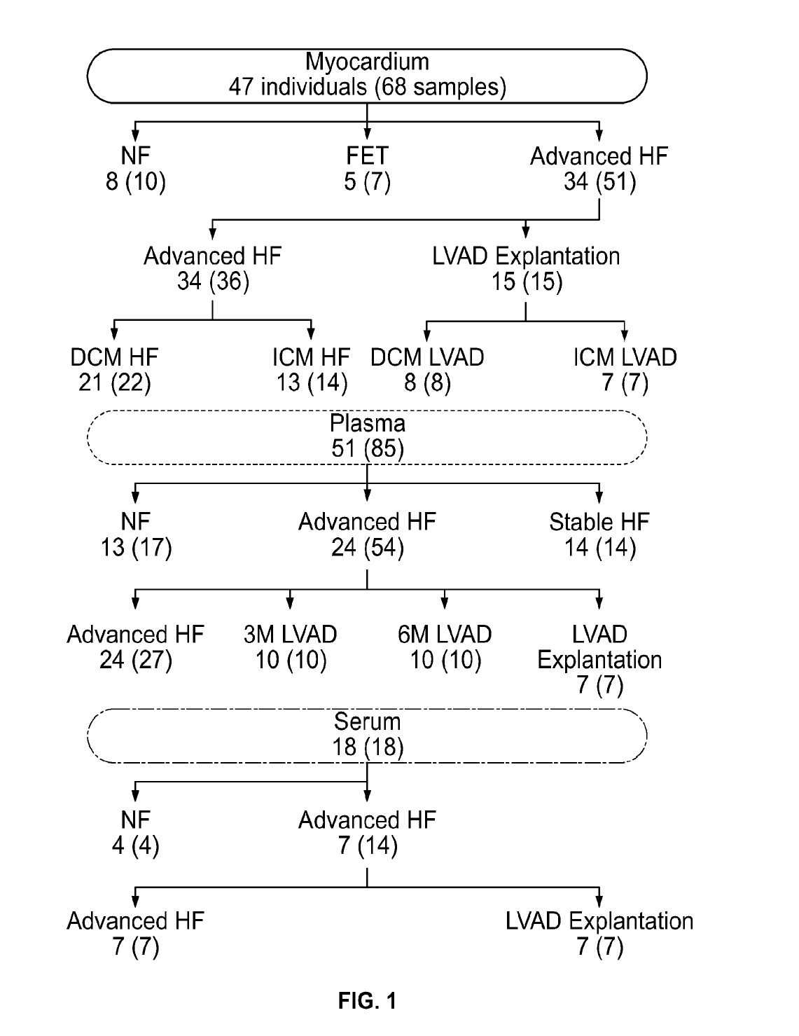

RNA was isolated from left-ventricular tissue samples from a total of 47 subjects; 21 patients with advanced HF due to dilated cardiomyopathy (DCM), 13 patients with advanced HF due to ischemic cardiomyopathy (ICM), 8 individuals without heart disease, and 5 fetuses (FET). The advanced HF samples were collected at the time of LVAD implantation (DCM/ICM HF) and LVAD explantation (DCM/ICM LVAD) during heart transplantation (FIG. 1—Number of individuals in each group and tissue with the number of samples shown in parentheses. NF: non-failing postnatal myocardium or plasma or serum from healthy volunteers; FET: fetal non-diseased myocardium; Advanced HF: advanced heart failure group at LVAD implantation (Advanced HF), 3 (3M LVAD) or 6 months (6M LVAD) after LVAD implantation and at LVAD explantation; Stable HF: stable heart failure.) The median total RNA yield was 0.5 μg per mg myocardium (interquartile range, IQR=0.2; RNA quantification and miRNA expression based on cistrons in all myocardial samples. (A and B) RNA yield (A) and miRNA content (B) in myocardial samples for the individual groups indicated. We obtained a median of 4.6 million miRNA reads per cardiac tissue sample (0.4-10.6 million), representing 67-93% of the total reads. In selected non-cardiac samples included for comparison, the median was 1.7 million miRNA reads (0.6-2.9 million), representing 37-99% of total reads. The myocardial miRNA content was 20 fmol per μg total RNA (IQR=9 fmol) and was calculated from the read ratios of all miRNA reads to spiked-in calibrator reads. The miRNA content was not significantly different between groups and comparable to other tissues. Farazi T. et al. (2011) Cancer Res 71:4443-4453.

To investigate myocardial miRNA expression changes, we combined the reads corresponding to miRNA genes organized in miRNA cistrons (Farazi T. et al. (2011) Cancer Res 71:4443-4453; Landgraf P et al. (2007) Cell 129:1401-1414); cistrons are labeled all lower case followed by the number of the founding member and the number of cistronic miRNAs in parentheses (FIG. 2). Forty-two miRNA cistrons changed in DCM (23 up, 19 down) and 54 cistrons changed in ICM (30 up, 24 down) HF compared to NF. Experiments with siRNAs or antagomirs (Krutzfeldt J et al. (2005). Nature 438:685-689) showed that only highly expressed miRNAs effectively repress target mRNAs. For simplicity of data presentation and discussion, we thus focused on regulatory miRNAs that contribute to ˜85% of sequencing reads per sample, corresponding to 15, 25, 21, and 28 miRNA cistrons in NF, FET, DCM HF and ICM HF, respectively (Tables 3-7). Williams Z et al. (2013) Proc Natl Acad Sci USA 110:4255-4260; Farazi T A et al. (2011) Cancer Res 71:4443-4453. Of these highly expressed cistrons ˜20% changed in DCM HF and ICM HF compared to NF ranging in absolute values from 1.4-fold for mir-1-1(4) to 2.9-fold for mir-221(2). The most highly expressed cistron mir-1-1(4) in myocardial tissue changed from an average read frequency of 25% in NF to 18% (1.4-fold) and 17% (1.6-fold) in DCM HF and ICM HF, respectively. While some of the differentially expressed cistrons were common to DCM HF and ICM HF, some were exclusive to ICM HF. The myomirs mir-208a(1), mir-208b(1), and mir-499(1) were unaltered in either DCM HF or ICM HF. Considering less abundant miRNA cistrons and their variation across sample groups, they were typically less than 4-fold, except for mir-216a(3) that increased 22-fold in DCM HF and a 47-fold in ICM HF compared to NF. Mir-216a(3) was at least 10-times higher expressed in HUV endothelial cells (HUVEC) possibly indicative of altered endothelial cell function in the heart. Finally, we did not observe significant changes in miRNA cistron expression comparing the patient-matched myocardial samples taken at the time of LVAD implantation and during explantation.

In FET, a total of 111 cistrons changed compared to NF (54 up, 57 down). In contrast to DCM and ICM HF, 60% of the highly expressed miRNAs in FET were differentially regulated at a higher magnitude than in failing myocardium. This was particularly the case for mir-29a(4). This cistron was expressed at 0.06% read frequency in FET and increased 90-fold to 5.6% in NF, while its expression was unchanged in HF. We observed a similar large difference in mir-29a(4) expression in skeletal muscle. Considering myomir expression, levels of mir-1-1(4) were reduced in FET versus NF, mirroring the changes in HF described above, however, mir-208a(1), mir-208b(1), and mir-499(1), all of which are located in introns of myosin genes, were lower by 2.6-, 4.0-, and 3.9-fold, respectively, and unaltered in HF.

(b) The Circulating Small RNA Pool Consists of miRNAs, and Fragments of tRNAs, and scRNAs

To determine whether myocardial miRNA expression changes translated into measurable changes in the circulating miRNA fraction, we isolated RNA from potassium-EDTA-treated plasma as well as serum samples from three cohorts representing different clinical stages of HF (FIG. 1): (1) healthy controls (NF, n=13); (2) patients with advanced HF with samples collected at LVAD implantation (advanced HF, n=24) and during routine outpatient visits after 3 (3M LVAD, n=10) or 6 months (6M LVAD, n=10), and at the time of LVAD explantation (n=7). Twelve of the 24 advanced HF samples were procured from patients for whom we generated myocardial miRNA profiles; (3) ambulatory patients with highly reduced left ventricular function stabilized with conventional pharmacologic therapy (stable HF, n=14). We sequenced small RNA cDNA libraries prepared from plasma total RNA from all patients of the three cohorts as well as serum total RNA from 18 individuals of cohorts 1 and 2 (serum-plasma pairs). The median recovery of total RNA was 30 ng/ml (IQR=17 ng/ml) for plasma and 69 ng/ml (IQR=70 ng/ml) for serum.

The circulating small RNA content was mainly miRNAs, and fragments of small cytoplasmic RNAs (scRNAs) and tRNAs. The average plasma and serum tRNA composition differed 47-fold and was 0.6% (IQR 0.9%) in plasma and 28% (IQR 33%) in serum while the scRNA content remained stable.

The serum samples had a median of 0.9 million miRNA reads (80,000-6 million) and the plasma samples 1.4 million (40.000-14 million The median miRNA content was 51 fmol/μg total RNA (IQR=26 fmol/μg) in serum, and due to the lower tRNA concentration higher in plasma with 116 fmol/μg total RNA (IQR=119 fmol/μg).

(c) The Circulating miRNA Profile in HF

The most abundant circulating miRNAs in healthy individuals probably originate from circulating blood cells (Williams Z et al. (2013) Proc Natl Acad Sci USA 110:4255-4260) and endothelial (HUVEC) cells, where they are highly expressed. In healthy individuals, only a few miRNAs known to be specifically expressed in solid tissues were among the top 85% sequence reads, including mir-122(1) from liver and mir-1-1(4) from muscle, but not the cardiac-specific myomirs. The combined myomir abundance in healthy individuals was less than 0.1%, however, it increased to over 1% in advanced HF patients. Myomirs displayed the biggest differences in levels among the 119 significantly changed miRNA cistrons (64 up, 55 down) in advanced HF patients compared to NF. The cardiac-specific myomirs mir-208a(1), mir-208b(1), mir-499(1), and the muscle-specific mir-1-1(4), and mir-133b(2) were 143-, 78-, 28-, 18-, and 21-fold higher, respectively, in advanced HF at LVAD implantation compared to NF. We also noted a 25-fold increase in mir-216a(3) in advanced HF, which at first sight paralleled a similar magnitude change in cardiac tissue. However, analysis of individual-paired samples and of absolute amounts suggested that the increase in circulating mir-216a(3) in advanced HF was not directly linked to the release of cardiac myomirs. More likely, endothelial cells, which express mir-216a(3) at higher levels than whole heart tissue, released it in response to advanced HF and its clinical management. Overall, 19 cistrons differed more than 5-fold in advanced HF compared to NF.

The data are summarized in Table 3 which shows the differences in the plasma levels in advanced heart failure (LVAD implantation as compared to healthy controls). Table 3 is an analysis of miRNA cistron abundance changes comparing plasma from patients with advanced heart failure (advanced HF; at LVAD implantation) to plasma from healthy volunteers (NF). The normalized read frequency is represented as a fraction. The false discovery rate (FDR) was calculated by the method of Benjamini and Hochberg.

The minus sign “−” in front of some numbers in the “Fold Change” column in Tables 3-7 indicates the level of the miRNA decreases in the sample of interest (e.g., advanced HF in Table 3, etc.).

TABLE 3
Differences in plasma levels in advanced heart failure
(LVAD implantation) compared to healthy controls

| | Normalized Read | | | | |
|---|---|---|---|---|---|
| | Frequency | | | | |
| miRNA | Advanced | Healthy | Fold | P | |
| Cistron | HF | Controls | Change | value | FDR |
| mir-208b(1) | 0.000332 | 0.000002 | 143.09 | 4.09E−17 | 9.56E−15 |
| mir-208a(1) | 0.000181 | 0.000002 | 78.20 | 2.75E−14 | 2.68E−12 |
| mir-499(1) | 0.000490 | 0.000017 | 28.20 | 8.77E−12 | 5.13E−10 |
| mir-216a(3) | 0.000044 | 0.000002 | 24.73 | 1.17E−08 | 2.68E−07 |
| mir-133b(2) | 0.000426 | 0.000020 | 21.11 | 1.37E−08 | 2.68E−07 |
| mir-1-1(4) | 0.011052 | 0.000626 | 17.65 | 3.14E−10 | 1.05E−08 |
| mir-95(1) | 0.000155 | 0.000010 | 15.15 | 1.05E−10 | 4.10E−09 |
| mir-488(1) | 0.000007 | 0.000001 | 13.03 | 1.58E−06 | 1.76E−05 |
| mir-3614(1) | 0.000005 | 0.000001 | 8.52 | 3.34E−04 | 1.45E−03 |
| mir-218-1(3) | 0.000035 | 0.000005 | 7.72 | 9.41E−05 | 5.79E−04 |
| mir-506(11) | 0.000019 | 0.000003 | 6.22 | 5.71E−05 | 3.93E−04 |
| mir-3158(1) | 0.000030 | 0.000005 | 6.20 | 3.36E−07 | 4.14E−06 |
| mir-15a(4) | 0.158742 | 0.027071 | 5.86 | 3.49E−11 | 1.63E−09 |
| mir-144(2) | 0.369400 | 0.063780 | 5.79 | 8.24E−06 | 7.71E−05 |
| mir-1247(1) | 0.000005 | 0.000001 | 5.74 | 8.33E−04 | 3.30E−03 |
| mir-378(1) | 0.005068 | 0.000900 | 5.63 | 7.74E−08 | 1.29E−06 |
| mir-455(1) | 0.000006 | 0.000001 | 5.05 | 8.23E−05 | 5.35E−04 |
| mir-887(1) | 0.000006 | 0.000001 | 4.96 | 9.33E−04 | 3.58E−03 |
| mir-195(2) | 0.001003 | 0.000206 | 4.88 | 1.43E−08 | 2.68E−07 |
| mir-2115(1) | 0.000004 | 0.000001 | 4.67 | 2.05E−03 | 6.75E−03 |
| mir-618(1) | 0.000006 | 0.000001 | 4.47 | 3.01E−03 | 9.52E−03 |
| mir-1180(1) | 0.000019 | 0.000004 | 4.46 | 1.27E−04 | 7.23E−04 |
| mir-10b(1) | 0.004800 | 0.001088 | 4.41 | 2.38E−04 | 1.18E−03 |
| mir-148a(1) | 0.026121 | 0.006542 | 3.99 | 1.31E−06 | 1.53E−05 |
| mir-193a(4) | 0.000604 | 0.000155 | 3.90 | 2.94E−06 | 3.13E−05 |
| mir-424(2) | 0.001932 | 0.000501 | 3.86 | 2.13E−07 | 2.93E−06 |
| mir-202(1) | 0.000005 | 0.000001 | 3.68 | 3.51E−02 | 7.46E−02 |
| mir-3909(1) | 0.000007 | 0.000002 | 3.67 | 1.82E−03 | 6.49E−03 |
| mir-3688(1) | 0.000012 | 0.000003 | 3.57 | 2.52E−04 | 1.18E−03 |
| mir-96(3) | 0.002088 | 0.000589 | 3.55 | 6.32E−06 | 6.17E−05 |
| mir-511-1(2) | 0.000027 | 0.000008 | 3.54 | 3.53E−03 | 1.09E−02 |
| mir-675(1) | 0.000005 | 0.000001 | 3.49 | 1.38E−02 | 3.47E−02 |
| mir-10a(1) | 0.003905 | 0.001243 | 3.14 | 1.26E−04 | 7.23E−04 |
| mir-550-1(2) | 0.000097 | 0.000035 | 2.76 | 5.25E−05 | 3.72E−04 |
| mir-1294(1) | 0.000014 | 0.000005 | 2.76 | 3.89E−03 | 1.18E−02 |
| mir-7-1(3) | 0.000782 | 0.000285 | 2.74 | 2.06E−05 | 1.72E−04 |
| mir-570(1) | 0.000014 | 0.000005 | 2.74 | 1.93E−03 | 6.66E−03 |
| mir-3143(1) | 0.000009 | 0.000003 | 2.72 | 1.53E−02 | 3.73E−02 |
| mir-486(1) | 0.053455 | 0.020214 | 2.64 | 1.63E−03 | 5.97E−03 |
| mir-143(2) | 0.008179 | 0.003187 | 2.57 | 5.53E−04 | 2.27E−03 |
| mir-1270-1(1) | 0.000007 | 0.000003 | 2.52 | 2.02E−03 | 6.75E−03 |
| mir-1270-2(1) | 0.000007 | 0.000003 | 2.52 | 2.02E−03 | 6.75E−03 |
| mir-651(1) | 0.000049 | 0.000019 | 2.51 | 1.71E−02 | 4.12E−02 |
| mir-3157(1) | 0.000006 | 0.000002 | 2.51 | 1.04E−02 | 2.72E−02 |
| mir-29a(4) | 0.012446 | 0.004993 | 2.49 | 2.96E−05 | 2.33E−04 |
| mir-3667(1) | 0.000006 | 0.000003 | 2.40 | 3.34E−02 | 7.23E−02 |
| mir-34b(2) | 0.000015 | 0.000006 | 2.32 | 4.62E−02 | 9.23E−02 |
| mir-210(1) | 0.000368 | 0.000164 | 2.24 | 4.64E−04 | 1.95E−03 |
| mir-204(1) | 0.000035 | 0.000016 | 2.20 | 2.69E−02 | 5.99E−02 |
| mir-3613(1) | 0.000281 | 0.000130 | 2.16 | 1.03E−02 | 2.72E−02 |
| mir-335(1) | 0.002363 | 0.001127 | 2.10 | 4.66E−04 | 1.95E−03 |
| mir-576(1) | 0.000097 | 0.000047 | 2.08 | 8.58E−03 | 2.33E−02 |
| mir-22(1) | 0.048472 | 0.023428 | 2.07 | 9.28E−04 | 3.58E−03 |
| mir-107(1) | 0.003404 | 0.001650 | 2.06 | 1.18E−02 | 3.04E−02 |
| mir-624(1) | 0.000020 | 0.000010 | 2.00 | 3.88E−02 | 8.03E−02 |
| mir-1908(1) | 0.000009 | 0.000004 | 1.95 | 4.56E−02 | 9.20E−02 |
| mir-188(8) | 0.001333 | 0.000705 | 1.89 | 1.03E−03 | 3.87E−03 |
| mir-320(1) | 0.014452 | 0.007738 | 1.87 | 5.80E−04 | 2.34E−03 |
| mir-874(1) | 0.000063 | 0.000036 | 1.76 | 4.81E−02 | 9.45E−02 |
| mir-26b(1) | 0.030145 | 0.017513 | 1.72 | 3.39E−02 | 7.28E−02 |
| mir-186(1) | 0.005610 | 0.003386 | 1.66 | 1.75E−02 | 4.14E−02 |
| mir-139(1) | 0.001186 | 0.000740 | 1.60 | 2.16E−02 | 4.95E−02 |
| mir-103-1(2) | 0.031211 | 0.020134 | 1.55 | 3.01E−02 | 6.65E−02 |
| mir-1306(1) | 0.000077 | 0.000051 | 1.52 | 3.65E−02 | 7.70E−02 |
| mir-98(13) | 0.050586 | 0.072644 | −1.44 | 2.08E−02 | 4.81E−02 |
| mir-574(1) | 0.000193 | 0.000298 | −1.54 | 1.50E−02 | 3.70E−02 |
| mir-1287(1) | 0.000010 | 0.000016 | −1.61 | 4.73E−02 | 9.39E−02 |
| mir-301a(2) | 0.000435 | 0.000715 | −1.64 | 1.95E−02 | 4.55E−02 |
| mir-196b(1) | 0.000051 | 0.000086 | −1.68 | 3.19E−03 | 9.96E−03 |
| mir-652(1) | 0.001076 | 0.001834 | −1.70 | 6.10E−03 | 1.72E−02 |
| mir-1468(1) | 0.000005 | 0.000009 | −1.73 | 4.38E−02 | 8.92E−02 |
| mir-766(1) | 0.000110 | 0.000195 | −1.77 | 1.72E−02 | 4.12E−02 |
| mir-155(1) | 0.000164 | 0.000297 | −1.81 | 4.83E−03 | 1.41E−02 |
| mir-1271(1) | 0.000011 | 0.000021 | −1.84 | 4.98E−03 | 1.44E−02 |
| mir-744(1) | 0.001341 | 0.002470 | −1.84 | 5.49E−03 | 1.57E−02 |
| mir-135a-1(3) | 0.005509 | 0.010243 | −1.86 | 1.86E−03 | 6.51E−03 |
| mir-3177(1) | 0.000008 | 0.000015 | −1.88 | 2.18E−02 | 4.96E−02 |
| mir-200a(3) | 0.000054 | 0.000103 | −1.89 | 3.18E−02 | 6.95E−02 |
| mir-1301(1) | 0.000159 | 0.000302 | −1.90 | 1.83E−03 | 6.49E−03 |
| mir-221(2) | 0.005760 | 0.010998 | −1.91 | 3.27E−04 | 1.44E−03 |
| mir-605(1) | 0.000003 | 0.000006 | −1.94 | 9.58E−03 | 2.58E−02 |
| mir-101-1(2) | 0.003248 | 0.006324 | −1.95 | 7.03E−03 | 1.94E−02 |
| mir-126(1) | 0.015158 | 0.030771 | −2.03 | 2.71E−04 | 1.24E−03 |
| mir-598(1) | 0.000040 | 0.000081 | −2.03 | 2.80E−03 | 8.98E−03 |
| mir-134(41) | 0.002723 | 0.005599 | −2.06 | 4.45E−03 | 1.32E−02 |
| mir-590(1) | 0.000101 | 0.000211 | −2.08 | 1.03E−02 | 2.72E−02 |
| mir-3130(1) | 0.000002 | 0.000003 | −2.12 | 3.70E−02 | 7.74E−02 |
| mir-491(1) | 0.000015 | 0.000031 | −2.14 | 6.99E−03 | 1.94E−02 |
| mir-4326(1) | 0.000005 | 0.000011 | −2.15 | 1.23E−02 | 3.13E−02 |
| mir-326(1) | 0.000094 | 0.000207 | −2.21 | 1.45E−02 | 3.61E−02 |
| mir-223(1) | 0.009023 | 0.019996 | −2.22 | 1.67E−04 | 8.70E−04 |
| mir-205(1) | 0.000008 | 0.000019 | −2.22 | 4.11E−02 | 8.44E−02 |
| mir-199b(1) | 0.001093 | 0.002494 | −2.28 | 4.05E−03 | 1.21E−02 |
| mir-1179(1) | 0.000004 | 0.000008 | −2.29 | 2.27E−03 | 7.37E−03 |
| mir-589(1) | 0.000021 | 0.000049 | −2.32 | 1.33E−04 | 7.42E−04 |
| mir-580(1) | 0.000001 | 0.000003 | −2.41 | 2.41E−02 | 5.42E−02 |
| mir-181a-1(4) | 0.002238 | 0.005547 | −2.48 | 2.67E−07 | 3.48E−06 |
| mir-28(1) | 0.000999 | 0.002514 | −2.52 | 2.99E−05 | 2.33E−04 |
| mir-552(1) | 0.000001 | 0.000003 | −2.52 | 1.25E−03 | 4.65E−03 |
| mir-3065(1) | 0.000006 | 0.000016 | −2.54 | 3.07E−04 | 1.38E−03 |
| mir-769(1) | 0.000079 | 0.000205 | −2.59 | 7.73E−05 | 5.17E−04 |
| mir-505(1) | 0.000057 | 0.000150 | −2.66 | 8.48E−05 | 5.36E−04 |
| mir-2355(1) | 0.000029 | 0.000077 | −2.66 | 5.25E−05 | 3.72E−04 |
| mir-127(8) | 0.000747 | 0.002018 | −2.70 | 2.50E−04 | 1.18E−03 |
| mir-199a-1(3) | 0.002524 | 0.006956 | −2.76 | 1.49E−04 | 7.92E−04 |
| mir-643(1) | 0.000002 | 0.000004 | −2.81 | 2.23E−04 | 1.13E−03 |
| mir-1296(1) | 0.000004 | 0.000012 | −2.99 | 1.37E−04 | 7.47E−04 |
| mir-671(1) | 0.000029 | 0.000091 | −3.16 | 1.93E−07 | 2.83E−06 |
| mir-328(1) | 0.000123 | 0.000400 | −3.25 | 1.78E−07 | 2.77E−06 |
| mir-370(1) | 0.000056 | 0.000190 | −3.41 | 1.88E−05 | 1.63E−04 |
| mir-190a(1) | 0.000016 | 0.000054 | −3.49 | 1.11E−04 | 6.63E−04 |
| mir-1307(1) | 0.000185 | 0.000667 | −3.61 | 4.60E−09 | 1.20E−07 |
| mir-551b(1) | 0.000004 | 0.000016 | −3.95 | 8.59E−06 | 7.73E−05 |
| mir-584(1) | 0.000361 | 0.001469 | −4.08 | 1.49E−08 | 2.68E−07 |
| mir-1277(1) | 0.000064 | 0.000262 | −4.09 | 2.46E−04 | 1.18E−03 |
| mir-181c(2) | 0.000091 | 0.000391 | −4.29 | 3.44E−14 | 2.68E−12 |
| mir-1250(1) | 0.000002 | 0.000012 | −4.91 | 5.34E−06 | 5.43E−05 |
| mir-375(1) | 0.000046 | 0.000265 | −5.75 | 4.67E−05 | 3.53E−04 |
| mir-1249(1) | 0.000001 | 0.000009 | −7.48 | 9.14E−10 | 2.67E−08 |
| mir-3138(1) | 0.000005 | 0.000008 | −1.58 | 5.15E−02 | 1.00E−01 |
| mir-33a(1) | 0.000119 | 0.000220 | −1.84 | 5.61E−02 | 1.08E−01 |
| mir-92b(1) | 0.000159 | 0.000090 | 1.77 | 5.70E−02 | 1.09E−01 |
| mir-3176(1) | 0.000004 | 0.000002 | 2.43 | 5.98E−02 | 1.14E−01 |
| mir-150(1) | 0.000658 | 0.001050 | −1.60 | 6.04E−02 | 1.14E−01 |
| mir-2277(1) | 0.000004 | 0.000007 | −1.65 | 6.11E−02 | 1.14E−01 |
| mir-3074(1) | 0.000006 | 0.000009 | −1.61 | 7.37E−02 | 1.37E−01 |
| mir-873(2) | 0.000007 | 0.000003 | 2.29 | 7.66E−02 | 1.41E−01 |
| mir-3617(1) | 0.000005 | 0.000010 | −1.92 | 7.82E−02 | 1.43E−01 |
| mir-340(1) | 0.002428 | 0.001563 | 1.55 | 8.12E−02 | 1.47E−01 |
| mir-184(1) | 0.000004 | 0.000001 | 2.74 | 8.23E−02 | 1.48E−01 |
| mir-625(1) | 0.000234 | 0.000362 | −1.55 | 8.70E−02 | 1.54E−01 |
| mir-641(1) | 0.000009 | 0.000015 | −1.72 | 8.71E−02 | 1.54E−01 |
| mir-2276(1) | 0.000003 | 0.000002 | 1.79 | 8.82E−02 | 1.55E−01 |
| mir-148b(1) | 0.008501 | 0.006137 | 1.39 | 9.70E−02 | 1.69E−01 |
| mir-132(2) | 0.000102 | 0.000144 | −1.40 | 1.06E−01 | 1.85E−01 |
| mir-140(1) | 0.005280 | 0.003796 | 1.39 | 1.10E−01 | 1.89E−01 |
| mir-888(6) | 0.000003 | 0.000001 | 2.49 | 1.17E−01 | 1.99E−01 |
| mir-342(1) | 0.001171 | 0.000885 | 1.32 | 1.34E−01 | 2.27E−01 |
| mir-9-1(3) | 0.000019 | 0.000028 | −1.43 | 1.35E−01 | 2.28E−01 |
| mir-498(46) | 0.000017 | 0.000009 | 1.81 | 1.41E−01 | 2.34E−01 |
| mir-224(2) | 0.000434 | 0.000619 | −1.43 | 1.41E−01 | 2.34E−01 |
| mir-17(12) | 0.038830 | 0.049538 | −1.28 | 1.43E−01 | 2.35E−01 |
| mir-190b(1) | 0.000008 | 0.000011 | −1.32 | 1.44E−01 | 2.35E−01 |
| mir-147(1) | 0.000002 | 0.000003 | −1.64 | 1.55E−01 | 2.50E−01 |
| mir-146a(1) | 0.012109 | 0.015599 | −1.29 | 1.55E−01 | 2.50E−01 |
| mir-610(1) | 0.000003 | 0.000002 | 1.84 | 1.57E−01 | 2.52E−01 |
| mir-130b(2) | 0.000713 | 0.000558 | 1.28 | 1.61E−01 | 2.56E−01 |
| mir-616(1) | 0.000007 | 0.000005 | 1.58 | 1.63E−01 | 2.58E−01 |
| mir-324(1) | 0.000347 | 0.000442 | −1.27 | 1.66E−01 | 2.60E−01 |
| mir-361(1) | 0.001512 | 0.001148 | 1.32 | 1.72E−01 | 2.68E−01 |
| let-7i(1) | 0.010117 | 0.013275 | −1.31 | 1.77E−01 | 2.74E−01 |
| mir-628(1) | 0.000080 | 0.000059 | 1.36 | 1.85E−01 | 2.85E−01 |
| mir-659(1) | 0.000002 | 0.000001 | 1.93 | 1.94E−01 | 2.97E−01 |
| mir-3136(1) | 0.000001 | 0.000002 | −1.71 | 2.04E−01 | 3.09E−01 |
| mir-151(1) | 0.009538 | 0.012459 | −1.31 | 2.04E−01 | 3.09E−01 |
| mir-3940(1) | 0.000006 | 0.000004 | 1.58 | 2.26E−01 | 3.40E−01 |
| mir-197(1) | 0.000586 | 0.000449 | 1.31 | 2.31E−01 | 3.44E−01 |
| mir-3140(1) | 0.000003 | 0.000002 | 1.62 | 2.34E−01 | 3.47E−01 |
| mir-138-1(2) | 0.000003 | 0.000004 | −1.39 | 2.37E−01 | 3.48E−01 |
| mir-128-1(2) | 0.000834 | 0.001068 | −1.28 | 2.56E−01 | 3.73E−01 |
| mir-3115(1) | 0.000002 | 0.000003 | −1.34 | 2.57E−01 | 3.73E−01 |
| mir-490(1) | 0.000006 | 0.000003 | 1.82 | 2.72E−01 | 3.93E−01 |
| mir-2116(1) | 0.000002 | 0.000003 | −1.35 | 2.82E−01 | 4.05E−01 |
| mir-449a(3) | 0.000003 | 0.000003 | −1.27 | 2.84E−01 | 4.05E−01 |
| mir-331(1) | 0.000090 | 0.000111 | −1.23 | 2.87E−01 | 4.07E−01 |
| mir-3120(1) | 0.000019 | 0.000015 | 1.31 | 2.96E−01 | 4.18E−01 |
| mir-339(1) | 0.000565 | 0.000676 | −1.20 | 3.05E−01 | 4.26E−01 |
| mir-30b(2) | 0.014074 | 0.016572 | −1.18 | 3.06E−01 | 4.26E−01 |
| mir-3124(1) | 0.000005 | 0.000003 | 1.55 | 3.09E−01 | 4.29E−01 |
| mir-3200(1) | 0.000006 | 0.000004 | 1.69 | 3.18E−01 | 4.38E−01 |
| mir-149(1) | 0.000002 | 0.000001 | 1.59 | 3.47E−01 | 4.71E−01 |
| mir-1256(1) | 0.000009 | 0.000007 | 1.30 | 3.48E−01 | 4.71E−01 |
| mir-423(1) | 0.006291 | 0.007429 | −1.18 | 3.49E−01 | 4.71E−01 |
| mir-124-1(3) | 0.000003 | 0.000004 | −1.56 | 3.50E−01 | 4.71E−01 |
| mir-296(1) | 0.000009 | 0.000006 | 1.49 | 3.56E−01 | 4.76E−01 |
| mir-141(2) | 0.000192 | 0.000237 | −1.23 | 3.97E−01 | 5.28E−01 |
| mir-122(1) | 0.002584 | 0.003319 | −1.28 | 4.01E−01 | 5.30E−01 |
| mir-3174(1) | 0.000002 | 0.000001 | 1.42 | 4.19E−01 | 5.51E−01 |
| mir-3611(1) | 0.000007 | 0.000005 | 1.45 | 4.49E−01 | 5.87E−01 |
| mir-556(1) | 0.000025 | 0.000031 | −1.23 | 4.79E−01 | 6.22E−01 |
| mir-1304(1) | 0.000030 | 0.000035 | −1.15 | 4.91E−01 | 6.29E−01 |
| mir-885(1) | 0.000017 | 0.000013 | 1.28 | 4.91E−01 | 6.29E−01 |
| mir-153-1(2) | 0.000013 | 0.000015 | −1.18 | 4.92E−01 | 6.29E−01 |
| mir-1255a(1) | 0.000007 | 0.000006 | 1.22 | 4.95E−01 | 6.29E−01 |
| mir-30a(4) | 0.014414 | 0.016051 | −1.11 | 4.99E−01 | 6.30E−01 |
| mir-2278(1) | 0.000002 | 0.000003 | −1.15 | 5.01E−01 | 6.30E−01 |
| mir-450a-1(4) | 0.000133 | 0.000111 | 1.19 | 5.12E−01 | 6.40E−01 |
| mir-130a(1) | 0.002522 | 0.002845 | −1.13 | 5.27E−01 | 6.56E−01 |
| mir-504(1) | 0.000003 | 0.000003 | −1.16 | 5.41E−01 | 6.70E−01 |
| mir-25(3) | 0.021928 | 0.019815 | 1.11 | 5.45E−01 | 6.71E−01 |
| mir-579(1) | 0.000005 | 0.000005 | −1.08 | 5.56E−01 | 6.75E−01 |
| mir-3605(1) | 0.000007 | 0.000008 | −1.10 | 5.59E−01 | 6.75E−01 |
| mir-99b(3) | 0.003293 | 0.003649 | −1.11 | 5.59E−01 | 6.75E−01 |
| mir-3928(1) | 0.000011 | 0.000010 | 1.16 | 5.62E−01 | 6.75E−01 |
| mir-3912(1) | 0.000005 | 0.000004 | 1.30 | 5.62E−01 | 6.75E−01 |
| mir-483(1) | 0.000016 | 0.000021 | −1.26 | 5.66E−01 | 6.75E−01 |
| mir-146b(1) | 0.000941 | 0.001053 | −1.12 | 5.70E−01 | 6.77E−01 |
| mir-129-1(2) | 0.000004 | 0.000004 | −1.17 | 5.85E−01 | 6.89E−01 |
| mir-642(1) | 0.000003 | 0.000003 | −1.12 | 5.86E−01 | 6.89E−01 |
| mir-330(1) | 0.000137 | 0.000119 | 1.16 | 5.89E−01 | 6.90E−01 |
| mir-32(1) | 0.000366 | 0.000314 | 1.17 | 5.99E−01 | 6.97E−01 |
| mir-3194(1) | 0.000003 | 0.000002 | 1.43 | 6.16E−01 | 7.14E−01 |
| mir-33b(1) | 0.000015 | 0.000017 | −1.14 | 6.33E−01 | 7.27E−01 |
| mir-152(1) | 0.000978 | 0.000888 | 1.10 | 6.34E−01 | 7.27E−01 |
| mir-484(1) | 0.002828 | 0.003049 | −1.08 | 6.53E−01 | 7.45E−01 |
| mir-3150(1) | 0.000002 | 0.000002 | −1.08 | 6.61E−01 | 7.51E−01 |
| mir-203(1) | 0.000066 | 0.000078 | −1.17 | 6.69E−01 | 7.56E−01 |
| mir-185(1) | 0.005220 | 0.004802 | 1.09 | 6.97E−01 | 7.84E−01 |
| mir-26a-1(2) | 0.046536 | 0.042685 | 1.09 | 7.06E−01 | 7.91E−01 |
| mir-3173(1) | 0.000007 | 0.000006 | 1.14 | 7.53E−01 | 8.37E−01 |
| mir-196a-1(3) | 0.000014 | 0.000012 | 1.14 | 7.55E−01 | 8.37E−01 |
| mir-708(1) | 0.000005 | 0.000005 | 1.04 | 7.69E−01 | 8.49E−01 |
| mir-489(2) | 0.000003 | 0.000002 | 1.30 | 7.91E−01 | 8.69E−01 |
| mir-142(1) | 0.014830 | 0.014335 | 1.03 | 8.19E−01 | 8.96E−01 |
| mir-627(1) | 0.000031 | 0.000033 | −1.07 | 8.28E−01 | 9.01E−01 |
| mir-219-1(2) | 0.000003 | 0.000003 | 1.05 | 8.37E−01 | 9.07E−01 |
| mir-3679(1) | 0.000002 | 0.000002 | 1.20 | 8.45E−01 | 9.11E−01 |
| mir-3127(1) | 0.000004 | 0.000004 | −1.07 | 8.59E−01 | 9.21E−01 |
| mir-3187(1) | 0.000010 | 0.000009 | 1.06 | 8.62E−01 | 9.21E−01 |
| mir-21(1) | 0.098820 | 0.095186 | 1.04 | 8.66E−01 | 9.21E−01 |
| mir-629(1) | 0.000184 | 0.000176 | 1.05 | 8.97E−01 | 9.47E−01 |
| mir-942(1) | 0.000047 | 0.000045 | 1.04 | 8.98E−01 | 9.47E−01 |
| mir-2110(1) | 0.000037 | 0.000035 | 1.05 | 9.03E−01 | 9.48E−01 |
| mir-338(1) | 0.000086 | 0.000084 | 1.03 | 9.22E−01 | 9.63E−01 |
| mir-1284(1) | 0.000004 | 0.000004 | 1.02 | 9.29E−01 | 9.66E−01 |
| mir-582(1) | 0.000013 | 0.000014 | −1.05 | 9.35E−01 | 9.68E−01 |
| mir-374a(4) | 0.003586 | 0.003612 | −1.01 | 9.57E−01 | 9.83E−01 |
| mir-760(1) | 0.000077 | 0.000077 | −1.01 | 9.60E−01 | 9.83E−01 |
| mir-191(2) | 0.018736 | 0.018530 | 1.01 | 9.62E−01 | 9.83E−01 |
| mir-34a(1) | 0.000054 | 0.000052 | 1.04 | 9.75E−01 | 9.92E−01 |
| mir-23a(6) | 0.039720 | 0.039747 | −1.00 | 9.85E−01 | 9.98E−01 |
| mir-192(4) | 0.001097 | 0.001089 | 1.01 | 9.96E−01 | 1.00E+00 |
| mir-3942(1) | 0.000003 | 0.000002 | 1.12 | 1.00E+00 | 1.00E+00 |
| mir-345(1) | 0.000122 | 0.000121 | 1.01 | 1.00E+00 | 1.00E+00 |

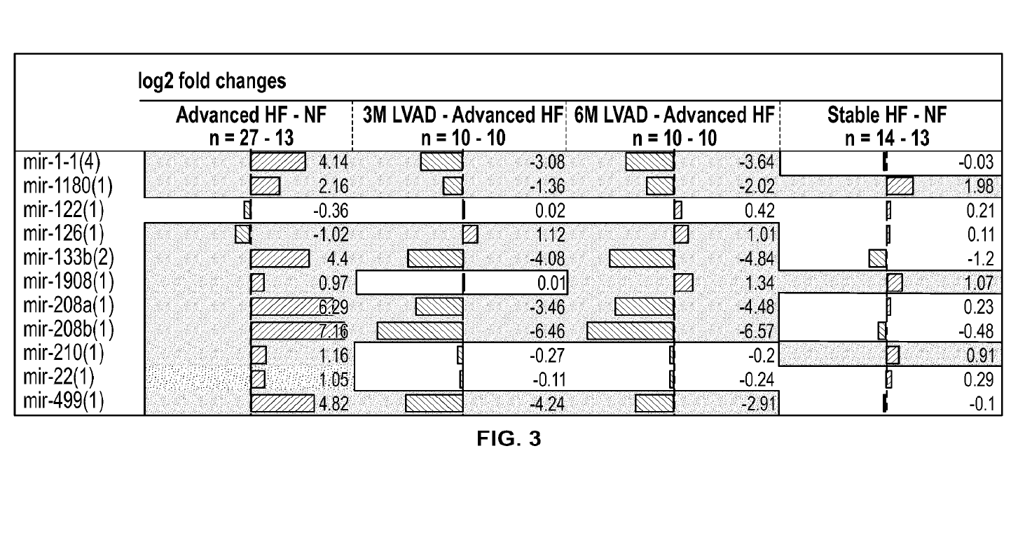

The miRNA changes in advanced HF reversed 3 and 6 months after LVAD implantation. The levels of the myomirs mir-208a(1), mir-208b(1), mir-499(1), and mir-1-1(4) dropped as early as 3 months after the initiation of LVAD support, approaching normal levels (Tables 4, 5 and FIG. 3). At LVAD explantation the myomir levels rose again with alterations comparable in magnitude to those observed at implantation.

Table 4 is an analysis of miRNA cistron abundance changes comparing plasma from patients with advanced heart failure 3 months after LVAD implantation (3 months LVAD) to plasma from the same patients (paired samples) at LVAD implantation (advanced HF). The normalized read frequency is represented as a fraction. The false discovery rate (FDR) was calculated by the method of Benjamini and Hochberg.

TABLE 4
Differences in plasma levels of reads originating from miRNA clusters
in patients treated 3 months with an LVAD compared to levels at
LVAD implantation

| | Normalized Read | | | | |
|---|---|---|---|---|---|
| | Frequency | | | | |
| miRNA | 3 months | Advance | Fold | | |
| Cistron | LVAD | HF | Change | P value | FDR |
| mir-208b(1) | 0.000002 | 0.000203 | −88.28 | 7.63E−21 | 1.62E−18 |
| mir-216a(3) | 0.000001 | 0.000050 | −48.65 | 1.65E−14 | 1.76E−12 |
| mir-499(1) | 0.000024 | 0.000463 | −18.94 | 1.79E−13 | 1.27E−11 |
| mir-133b(2) | 0.000014 | 0.000236 | −16.93 | 1.35E−09 | 7.17E−08 |
| mir-1277(1) | 0.000688 | 0.000058 | 11.79 | 4.92E−09 | 2.10E−07 |
| mir-95(1) | 0.000015 | 0.000158 | −10.21 | 7.49E−09 | 2.66E−07 |
| mir-193a(4) | 0.000076 | 0.000565 | −7.40 | 1.08E−08 | 3.27E−07 |
| mir-208a(1) | 0.000009 | 0.000099 | −11.03 | 1.92E−08 | 5.11E−07 |
| mir-190a(1) | 0.000117 | 0.000014 | 8.50 | 2.84E−07 | 6.30E−06 |
| mir-34b(2) | 0.000001 | 0.000016 | −11.71 | 2.96E−07 | 6.30E−06 |
| mir-511-1(2) | 0.000004 | 0.000035 | −8.73 | 7.35E−07 | 1.42E−05 |
| mir-1-1(4) | 0.001197 | 0.010096 | −8.43 | 9.56E−07 | 1.70E−05 |
| mir-33a(1) | 0.000632 | 0.000084 | 7.55 | 1.08E−06 | 1.77E−05 |
| mir-10b(1) | 0.000404 | 0.001991 | −4.93 | 2.36E−06 | 3.59E−05 |
| mir-378(1) | 0.000618 | 0.002664 | −4.31 | 7.50E−06 | 1.06E−04 |
| mir-218-1(3) | 0.000006 | 0.000045 | −7.10 | 1.70E−05 | 2.27E−04 |
| mir-10a(1) | 0.000715 | 0.002863 | −4.00 | 3.52E−05 | 4.42E−04 |
| mir-498(46) | 0.000004 | 0.000019 | −5.32 | 3.99E−05 | 4.73E−04 |
| mir-144(2) | 0.066243 | 0.243184 | −3.67 | 8.13E−05 | 9.11E−04 |
| mir-326(1) | 0.000264 | 0.000067 | 3.97 | 1.52E−04 | 1.62E−03 |
| mir-582(1) | 0.000003 | 0.000016 | −4.78 | 1.65E−04 | 1.67E−03 |
| mir-3158(1) | 0.000006 | 0.000024 | −4.26 | 1.93E−04 | 1.87E−03 |
| mir-486(1) | 0.009198 | 0.029173 | −3.17 | 2.87E−04 | 2.66E−03 |
| mir-199a-1(3) | 0.009282 | 0.002778 | 3.34 | 4.31E−04 | 3.83E−03 |
| mir-3617(1) | 0.000011 | 0.000002 | 4.44 | 6.25E−04 | 5.33E−03 |
| mir-127(8) | 0.002132 | 0.000707 | 3.02 | 8.17E−04 | 6.69E−03 |
| mir-766(1) | 0.000309 | 0.000100 | 3.09 | 8.60E−04 | 6.69E−03 |
| mir-195(2) | 0.000299 | 0.000914 | −3.06 | 8.79E−04 | 6.69E−03 |
| mir-505(1) | 0.000188 | 0.000061 | 3.07 | 9.78E−04 | 7.18E−03 |
| mir-181c(2) | 0.000406 | 0.000135 | 3.02 | 1.09E−03 | 7.72E−03 |
| mir-143(2) | 0.002127 | 0.006147 | −2.89 | 1.15E−03 | 7.93E−03 |
| mir-96(3) | 0.000406 | 0.001118 | −2.75 | 1.28E−03 | 8.53E−03 |
| mir-135a-1(3) | 0.016578 | 0.005940 | 2.79 | 1.43E−03 | 9.20E−03 |
| mir-301a(2) | 0.001180 | 0.000419 | 2.82 | 1.86E−03 | 1.16E−02 |
| mir-625(1) | 0.000645 | 0.000243 | 2.66 | 2.02E−03 | 1.23E−02 |
| mir-15a(4) | 0.041668 | 0.117084 | −2.81 | 2.13E−03 | 1.26E−02 |
| mir-199b(1) | 0.002957 | 0.001061 | 2.79 | 2.38E−03 | 1.37E−02 |
| mir-455(1) | 0.000001 | 0.000005 | −4.69 | 2.63E−03 | 1.47E−02 |
| mir-328(1) | 0.000400 | 0.000140 | 2.85 | 2.73E−03 | 1.49E−02 |
| mir-671(1) | 0.000097 | 0.000035 | 2.76 | 2.91E−03 | 1.50E−02 |
| mir-1307(1) | 0.000756 | 0.000266 | 2.84 | 2.92E−03 | 1.50E−02 |
| mir-28(1) | 0.002294 | 0.000903 | 2.54 | 2.95E−03 | 1.50E−02 |
| mir-424(2) | 0.000807 | 0.002052 | −2.54 | 3.09E−03 | 1.53E−02 |
| mir-2355(1) | 0.000106 | 0.000036 | 2.90 | 3.21E−03 | 1.53E−02 |
| mir-598(1) | 0.000102 | 0.000036 | 2.83 | 3.22E−03 | 1.53E−02 |
| mir-3909(1) | 0.000002 | 0.000008 | −3.53 | 3.77E−03 | 1.75E−02 |
| mir-584(1) | 0.001013 | 0.000371 | 2.73 | 4.14E−03 | 1.87E−02 |
| mir-375(1) | 0.000058 | 0.000019 | 3.00 | 4.41E−03 | 1.95E−02 |
| mir-134(41) | 0.006037 | 0.002494 | 2.42 | 4.89E−03 | 2.12E−02 |
| mir-29a(4) | 0.005062 | 0.012008 | −2.37 | 6.90E−03 | 2.94E−02 |
| mir-1301(1) | 0.000460 | 0.000200 | 2.30 | 8.05E−03 | 3.36E−02 |
| mir-223(1) | 0.033965 | 0.013542 | 2.51 | 8.53E−03 | 3.49E−02 |
| mir-33b(1) | 0.000036 | 0.000010 | 3.52 | 9.82E−03 | 3.95E−02 |
| mir-3177(1) | 0.000016 | 0.000006 | 2.87 | 1.00E−02 | 3.95E−02 |
| mir-148a(1) | 0.007186 | 0.015937 | −2.22 | 1.03E−02 | 3.98E−02 |
| mir-204(1) | 0.000018 | 0.000046 | −2.56 | 1.22E−02 | 4.64E−02 |
| mir-126(1) | 0.041692 | 0.019118 | 2.18 | 1.47E−02 | 5.48E−02 |
| mir-1287(1) | 0.000021 | 0.000009 | 2.45 | 1.74E−02 | 6.38E−02 |
| mir-1250(1) | 0.000011 | 0.000004 | 2.76 | 1.83E−02 | 6.60E−02 |
| mir-556(1) | 0.000054 | 0.000023 | 2.32 | 1.87E−02 | 6.64E−02 |
| mir-551b(1) | 0.000020 | 0.000007 | 2.70 | 1.99E−02 | 6.87E−02 |
| mir-3065(1) | 0.000017 | 0.000007 | 2.50 | 2.00E−02 | 6.87E−02 |
| mir-370(1) | 0.000247 | 0.000110 | 2.24 | 2.14E−02 | 7.23E−02 |
| mir-506(11) | 0.000009 | 0.000025 | −2.85 | 2.18E−02 | 7.24E−02 |
| mir-1180(1) | 0.000006 | 0.000014 | −2.57 | 2.27E−02 | 7.44E−02 |
| mir-331(1) | 0.000194 | 0.000094 | 2.07 | 2.48E−02 | 8.00E−02 |
| mir-3138(1) | 0.000013 | 0.000005 | 2.60 | 2.54E−02 | 8.07E−02 |
| mir-616(1) | 0.000011 | 0.000005 | 2.41 | 2.63E−02 | 8.25E−02 |
| mir-1296(1) | 0.000011 | 0.000004 | 2.68 | 2.75E−02 | 8.48E−02 |
| mir-130a(1) | 0.004685 | 0.002396 | 1.96 | 3.26E−02 | 9.93E−02 |
| mir-185(1) | 0.009596 | 0.005041 | 1.90 | 3.58E−02 | 1.07E−01 |
| mir-3688(1) | 0.000004 | 0.000009 | −2.41 | 3.72E−02 | 1.10E−01 |
| mir-643(1) | 0.000005 | 0.000002 | 2.80 | 4.06E−02 | 1.17E−01 |
| let-7i(1) | 0.018280 | 0.009542 | 1.92 | 4.06E−02 | 1.17E−01 |
| mir-570(1) | 0.000007 | 0.000015 | −2.14 | 4.26E−02 | 1.21E−01 |
| mir-708(1) | 0.000002 | 0.000005 | −2.83 | 4.83E−02 | 1.35E−01 |
| mir-98(13) | 0.097199 | 0.053033 | 1.83 | 5.03E−02 | 1.39E−01 |
| mir-192(4) | 0.000540 | 0.000983 | −1.82 | 5.10E−02 | 1.39E−01 |
| mir-1179(1) | 0.000009 | 0.000004 | 2.46 | 5.46E−02 | 1.47E−01 |
| mir-550-1(2) | 0.000042 | 0.000078 | −1.85 | 5.79E−02 | 1.54E−01 |
| mir-221(2) | 0.014618 | 0.007898 | 1.85 | 6.01E−02 | 1.58E−01 |
| mir-146b(1) | 0.001412 | 0.000800 | 1.76 | 6.46E−02 | 1.68E−01 |
| mir-885(1) | 0.000007 | 0.000013 | −1.93 | 6.82E−02 | 1.75E−01 |
| mir-155(1) | 0.000299 | 0.000164 | 1.83 | 7.03E−02 | 1.77E−01 |
| mir-203(1) | 0.000013 | 0.000028 | −2.14 | 7.05E−02 | 1.77E−01 |
| mir-652(1) | 0.002207 | 0.001269 | 1.74 | 7.38E−02 | 1.83E−01 |
| mir-150(1) | 0.000387 | 0.000671 | −1.73 | 7.61E−02 | 1.86E−01 |
| mir-590(1) | 0.000261 | 0.000142 | 1.84 | 8.17E−02 | 1.98E−01 |
| mir-374a(4) | 0.005271 | 0.003052 | 1.73 | 8.51E−02 | 2.04E−01 |
| mir-146a(1) | 0.019333 | 0.011570 | 1.67 | 8.75E−02 | 2.07E−01 |
| mir-873(2) | 0.000003 | 0.000008 | −2.20 | 9.32E−02 | 2.18E−01 |
| mir-330(1) | 0.000215 | 0.000126 | 1.71 | 9.95E−02 | 2.29E−01 |
| mir-1304(1) | 0.000038 | 0.000022 | 1.75 | 9.98E−02 | 2.29E−01 |
| mir-423(1) | 0.011165 | 0.006664 | 1.68 | 1.01E−01 | 2.30E−01 |
| mir-181a-1(4) | 0.003820 | 0.002315 | 1.65 | 1.03E−01 | 2.31E−01 |
| mir-589(1) | 0.000040 | 0.000023 | 1.72 | 1.04E−01 | 2.31E−01 |
| mir-224(2) | 0.000698 | 0.000411 | 1.70 | 1.07E−01 | 2.35E−01 |
| mir-296(1) | 0.000005 | 0.000009 | −1.94 | 1.08E−01 | 2.35E−01 |
| mir-744(1) | 0.002300 | 0.001383 | 1.66 | 1.09E−01 | 2.35E−01 |
| mir-17(12) | 0.048947 | 0.030412 | 1.61 | 1.21E−01 | 2.56E−01 |
| mir-7-1(3) | 0.000406 | 0.000651 | −1.60 | 1.22E−01 | 2.56E−01 |
| mir-1271(1) | 0.000023 | 0.000013 | 1.74 | 1.24E−01 | 2.56E−01 |
| mir-188(8) | 0.000686 | 0.001087 | −1.59 | 1.24E−01 | 2.56E−01 |
| mir-491(1) | 0.000032 | 0.000019 | 1.72 | 1.33E−01 | 2.71E−01 |
| mir-335(1) | 0.001367 | 0.002200 | −1.61 | 1.39E−01 | 2.82E−01 |
| mir-196b(1) | 0.000094 | 0.000058 | 1.62 | 1.48E−01 | 2.97E−01 |
| mir-339(1) | 0.000866 | 0.000563 | 1.54 | 1.54E−01 | 3.07E−01 |
| mir-1468(1) | 0.000006 | 0.000003 | 1.91 | 1.56E−01 | 3.07E−01 |
| mir-129-1(2) | 0.000002 | 0.000005 | −1.98 | 1.64E−01 | 3.20E−01 |
| mir-324(1) | 0.000666 | 0.000437 | 1.52 | 1.72E−01 | 3.34E−01 |
| mir-641(1) | 0.000014 | 0.000008 | 1.69 | 1.77E−01 | 3.39E−01 |
| mir-92b(1) | 0.000062 | 0.000095 | −1.55 | 1.80E−01 | 3.39E−01 |
| mir-128-1(2) | 0.001187 | 0.000776 | 1.53 | 1.80E−01 | 3.39E−01 |
| mir-490(1) | 0.000009 | 0.000005 | 1.75 | 1.87E−01 | 3.49E−01 |
| mir-605(1) | 0.000007 | 0.000004 | 1.80 | 1.95E−01 | 3.61E−01 |
| mir-642(1) | 0.000002 | 0.000004 | −1.82 | 2.15E−01 | 3.93E−01 |
| mir-3200(1) | 0.000002 | 0.000005 | −1.89 | 2.16E−01 | 3.93E−01 |
| mir-205(1) | 0.000005 | 0.000009 | −1.75 | 2.21E−01 | 4.00E−01 |
| mir-21(1) | 0.110708 | 0.076820 | 1.44 | 2.42E−01 | 4.28E−01 |
| mir-2277(1) | 0.000009 | 0.000005 | 1.62 | 2.43E−01 | 4.28E−01 |
| mir-610(1) | 0.000002 | 0.000003 | −1.87 | 2.43E−01 | 4.28E−01 |
| mir-1256(1) | 0.000013 | 0.000008 | 1.57 | 2.55E−01 | 4.34E−01 |
| mir-1294(1) | 0.000010 | 0.000015 | −1.55 | 2.56E−01 | 4.34E−01 |
| mir-140(1) | 0.003335 | 0.004701 | −1.41 | 2.56E−01 | 4.34E−01 |
| mir-504(1) | 0.000004 | 0.000002 | 1.82 | 2.56E−01 | 4.34E−01 |
| mir-3912(1) | 0.000007 | 0.000004 | 1.62 | 2.57E−01 | 4.34E−01 |
| mir-574(1) | 0.000362 | 0.000258 | 1.40 | 2.66E−01 | 4.45E−01 |
| mir-4326(1) | 0.000010 | 0.000006 | 1.55 | 2.67E−01 | 4.45E−01 |
| mir-449a(3) | 0.000004 | 0.000002 | 1.69 | 2.73E−01 | 4.50E−01 |
| mir-3613(1) | 0.000166 | 0.000235 | −1.42 | 2.83E−01 | 4.64E−01 |
| mir-651(1) | 0.000025 | 0.000037 | −1.46 | 2.87E−01 | 4.66E−01 |
| mir-624(1) | 0.000016 | 0.000023 | −1.45 | 3.03E−01 | 4.86E−01 |
| mir-769(1) | 0.000121 | 0.000086 | 1.41 | 3.03E−01 | 4.86E−01 |
| mir-30b(2) | 0.015123 | 0.011194 | 1.35 | 3.30E−01 | 5.24E−01 |
| mir-3176(1) | 0.000002 | 0.000004 | −1.58 | 3.41E−01 | 5.37E−01 |
| mir-107(1) | 0.002602 | 0.003495 | −1.34 | 3.46E−01 | 5.37E−01 |
| mir-151(1) | 0.010410 | 0.007787 | 1.34 | 3.46E−01 | 5.37E−01 |
| mir-579(1) | 0.000007 | 0.000004 | 1.51 | 3.48E−01 | 5.37E−01 |
| mir-153-1(2) | 0.000025 | 0.000018 | 1.42 | 3.51E−01 | 5.39E−01 |
| mir-3679(1) | 0.000004 | 0.000003 | 1.56 | 3.64E−01 | 5.54E−01 |
| mir-191(2) | 0.025140 | 0.019115 | 1.32 | 3.70E−01 | 5.56E−01 |
| mir-3173(1) | 0.000005 | 0.000007 | −1.45 | 3.71E−01 | 5.56E−01 |
| mir-142(1) | 0.021173 | 0.016173 | 1.31 | 3.77E−01 | 5.62E−01 |
| mir-26a-1(2) | 0.061716 | 0.047140 | 1.31 | 3.90E−01 | 5.77E−01 |
| mir-132(2) | 0.000074 | 0.000098 | −1.32 | 3.98E−01 | 5.84E−01 |
| mir-219-1(2) | 0.000004 | 0.000003 | 1.50 | 4.03E−01 | 5.88E−01 |
| mir-627(1) | 0.000041 | 0.000031 | 1.31 | 4.18E−01 | 6.06E−01 |
| mir-342(1) | 0.000808 | 0.001028 | −1.27 | 4.26E−01 | 6.13E−01 |
| mir-576(1) | 0.000087 | 0.000112 | −1.29 | 4.33E−01 | 6.19E−01 |
| mir-760(1) | 0.000123 | 0.000094 | 1.31 | 4.52E−01 | 6.42E−01 |
| mir-3942(1) | 0.000003 | 0.000002 | 1.45 | 4.58E−01 | 6.45E−01 |
| mir-200a(3) | 0.000055 | 0.000043 | 1.27 | 4.73E−01 | 6.59E−01 |
| mir-450a-1(4) | 0.000138 | 0.000175 | −1.26 | 4.74E−01 | 6.59E−01 |
| mir-152(1) | 0.001014 | 0.000811 | 1.25 | 4.77E−01 | 6.59E−01 |
| mir-23a(6) | 0.051954 | 0.042373 | 1.23 | 4.97E−01 | 6.80E−01 |
| mir-2116(1) | 0.000002 | 0.000003 | −1.43 | 4.98E−01 | 6.80E−01 |
| mir-1306(1) | 0.000087 | 0.000070 | 1.24 | 5.08E−01 | 6.89E−01 |
| mir-874(1) | 0.000053 | 0.000066 | −1.24 | 5.11E−01 | 6.89E−01 |
| mir-138-1(2) | 0.000003 | 0.000002 | 1.37 | 5.33E−01 | 7.10E−01 |
| mir-196a-1(3) | 0.000012 | 0.000015 | −1.25 | 5.33E−01 | 7.10E−01 |
| mir-210(1) | 0.000288 | 0.000348 | −1.21 | 5.37E−01 | 7.11E−01 |
| mir-34a(1) | 0.000050 | 0.000062 | −1.23 | 5.43E−01 | 7.14E−01 |
| mir-3940(1) | 0.000005 | 0.000006 | −1.28 | 5.48E−01 | 7.16E−01 |
| mir-3127(1) | 0.000004 | 0.000003 | 1.30 | 5.62E−01 | 7.30E−01 |
| mir-25(3) | 0.014563 | 0.017320 | −1.19 | 5.70E−01 | 7.35E−01 |
| mir-1255a(1) | 0.000011 | 0.000009 | 1.24 | 5.74E−01 | 7.37E−01 |
| mir-484(1) | 0.003007 | 0.002532 | 1.19 | 5.87E−01 | 7.44E−01 |
| mir-3157(1) | 0.000005 | 0.000004 | 1.28 | 5.87E−01 | 7.44E−01 |
| mir-3140(1) | 0.000003 | 0.000004 | −1.27 | 6.04E−01 | 7.60E−01 |
| mir-3150(1) | 0.000003 | 0.000002 | 1.31 | 6.07E−01 | 7.60E−01 |
| mir-2110(1) | 0.000048 | 0.000041 | 1.18 | 6.26E−01 | 7.79E−01 |
| mir-942(1) | 0.000055 | 0.000047 | 1.17 | 6.38E−01 | 7.90E−01 |
| mir-30a(4) | 0.014782 | 0.012746 | 1.16 | 6.43E−01 | 7.92E−01 |
| mir-2276(1) | 0.000002 | 0.000003 | −1.26 | 6.48E−01 | 7.94E−01 |
| mir-32(1) | 0.000328 | 0.000283 | 1.16 | 6.62E−01 | 8.06E−01 |
| mir-99b(3) | 0.003837 | 0.003365 | 1.14 | 6.69E−01 | 8.09E−01 |
| mir-3120(1) | 0.000023 | 0.000020 | 1.15 | 6.92E−01 | 8.28E−01 |
| mir-3605(1) | 0.000008 | 0.000007 | 1.16 | 7.01E−01 | 8.28E−01 |
| mir-101-1(2) | 0.003973 | 0.004516 | −1.14 | 7.02E−01 | 8.28E−01 |
| mir-1270-1(1) | 0.000005 | 0.000004 | 1.17 | 7.08E−01 | 8.28E−01 |
| mir-1270-2(1) | 0.000005 | 0.000004 | 1.17 | 7.08E−01 | 8.28E−01 |
| mir-148b(1) | 0.007161 | 0.008048 | −1.12 | 7.08E−01 | 8.28E−01 |
| mir-9-1(3) | 0.000026 | 0.000023 | 1.13 | 7.23E−01 | 8.40E−01 |
| mir-186(1) | 0.005922 | 0.005313 | 1.11 | 7.26E−01 | 8.40E−01 |
| mir-483(1) | 0.000011 | 0.000013 | −1.14 | 7.39E−01 | 8.48E−01 |
| mir-3124(1) | 0.000003 | 0.000003 | 1.17 | 7.41E−01 | 8.48E−01 |
| mir-139(1) | 0.001242 | 0.001368 | −1.10 | 7.57E−01 | 8.62E−01 |
| mir-3074(1) | 0.000010 | 0.000009 | 1.12 | 7.66E−01 | 8.66E−01 |
| mir-345(1) | 0.000151 | 0.000138 | 1.10 | 7.70E−01 | 8.66E−01 |
| mir-3611(1) | 0.000007 | 0.000008 | −1.13 | 7.72E−01 | 8.66E−01 |
| mir-22(1) | 0.038970 | 0.042131 | −1.08 | 7.99E−01 | 8.91E−01 |
| mir-141(2) | 0.000157 | 0.000146 | 1.08 | 8.11E−01 | 8.93E−01 |
| mir-3143(1) | 0.000008 | 0.000007 | 1.10 | 8.18E−01 | 8.93E−01 |
| mir-3928(1) | 0.000011 | 0.000010 | 1.09 | 8.18E−01 | 8.93E−01 |
| mir-3136(1) | 0.000002 | 0.000002 | 1.13 | 8.19E−01 | 8.93E−01 |
| mir-190b(1) | 0.000010 | 0.000011 | −1.09 | 8.22E−01 | 8.93E−01 |
| mir-130b(2) | 0.000818 | 0.000769 | 1.06 | 8.44E−01 | 9.11E−01 |
| mir-629(1) | 0.000173 | 0.000162 | 1.06 | 8.47E−01 | 9.11E−01 |
| mir-340(1) | 0.001806 | 0.001712 | 1.06 | 8.62E−01 | 9.23E−01 |
| mir-3187(1) | 0.000008 | 0.000009 | −1.06 | 8.70E−01 | 9.26E−01 |
| mir-628(1) | 0.000082 | 0.000087 | −1.05 | 8.76E−01 | 9.28E−01 |
| mir-197(1) | 0.000481 | 0.000501 | −1.04 | 8.96E−01 | 9.43E−01 |
| mir-361(1) | 0.001318 | 0.001268 | 1.04 | 8.99E−01 | 9.43E−01 |
| mir-3667(1) | 0.000004 | 0.000004 | −1.06 | 9.09E−01 | 9.49E−01 |
| mir-26b(1) | 0.026574 | 0.025658 | 1.04 | 9.14E−01 | 9.50E−01 |
| mir-103-1(2) | 0.032236 | 0.032864 | −1.02 | 9.49E−01 | 9.81E−01 |
| mir-122(1) | 0.002331 | 0.002294 | 1.02 | 9.63E−01 | 9.88E−01 |
| mir-147(1) | 0.000003 | 0.000003 | −1.02 | 9.65E−01 | 9.88E−01 |
| mir-3194(1) | 0.000005 | 0.000005 | 1.03 | 9.69E−01 | 9.88E−01 |
| mir-1284(1) | 0.000005 | 0.000005 | −1.01 | 9.77E−01 | 9.91E−01 |
| mir-338(1) | 0.000117 | 0.000116 | 1.00 | 9.89E−01 | 9.94E−01 |
| mir-1908(1) | 0.000010 | 0.000010 | 1.00 | 9.93E−01 | 9.94E−01 |
| mir-320(1) | 0.014432 | 0.014401 | 1.00 | 9.94E−01 | 9.94E−01 |

Table 5 is an analysis of miRNA cistron abundance changes comparing plasma from patients with advanced heart failure 6 months after LVAD implantation (6 months LVAD) to plasma from the same patients (paired samples) at LVAD implantation (advanced HF). The normalized read frequency is represented as a fraction. The false discovery rate (FDR) was calculated by the method of Benjamini and Hochberg.

TABLE 5
Differences in the plasma levels of reads originating from miRNA
cistron in patients treated 6 months with an LVAD compared to
levels at LVAD implantation

| | Normalized Read | | | | |
|---|---|---|---|---|---|
| | Frequency | | | | |
| miRNA | 6 months | Advance | Fold | | |
| Cistron | LVAD | HF | Change | P value | FDR |
| mir-208b(1) | 0.000002 | 0.000145 | −95.23 | 3.60E−36 | 7.56E−34 |
| mir-133b(2) | 0.000012 | 0.000343 | −28.56 | 5.19E−23 | 5.45E−21 |
| mir-95(1) | 0.000005 | 0.000109 | −23.34 | 1.09E−22 | 7.62E−21 |
| mir-208a(1) | 0.000009 | 0.000193 | −22.39 | 8.90E−19 | 4.67E−17 |
| mir-218-1(3) | 0.000001 | 0.000023 | −23.83 | 1.66E−14 | 6.96E−13 |
| mir-1-1(4) | 0.000517 | 0.006459 | −12.49 | 1.82E−13 | 6.37E−12 |
| mir-216a(3) | 0.000001 | 0.000019 | −15.51 | 1.33E−12 | 3.98E−11 |
| mir-10b(1) | 0.000273 | 0.001710 | −6.27 | 3.55E−11 | 9.32E−10 |
| mir-499(1) | 0.000020 | 0.000153 | −7.51 | 7.60E−11 | 1.77E−09 |
| mir-1277(1) | 0.000337 | 0.000048 | 6.96 | 2.85E−10 | 5.99E−09 |
| mir-193a(4) | 0.000070 | 0.000372 | −5.30 | 9.74E−10 | 1.86E−08 |
| mir-378(1) | 0.000504 | 0.002384 | −4.73 | 2.36E−09 | 4.13E−08 |
| mir-96(3) | 0.000337 | 0.001578 | −4.68 | 3.65E−09 | 5.89E−08 |
| mir-1307(1) | 0.000648 | 0.000136 | 4.77 | 5.23E−09 | 7.84E−08 |
| mir-34b(2) | 0.000001 | 0.000012 | −8.57 | 1.53E−08 | 2.15E−07 |
| mir-144(2) | 0.088538 | 0.408227 | −4.61 | 5.42E−08 | 7.12E−07 |
| mir-10a(1) | 0.000397 | 0.001723 | −4.34 | 7.15E−08 | 8.84E−07 |
| mir-15a(4) | 0.031394 | 0.126240 | −4.02 | 9.68E−08 | 1.13E−06 |
| mir-190a(1) | 0.000095 | 0.000019 | 4.98 | 2.70E−07 | 2.99E−06 |
| mir-181c(2) | 0.000377 | 0.000099 | 3.81 | 5.50E−07 | 5.61E−06 |
| mir-486(1) | 0.009317 | 0.035534 | −3.81 | 5.61E−07 | 5.61E−06 |
| mir-33a(1) | 0.000476 | 0.000105 | 4.55 | 8.55E−07 | 8.16E−06 |
| mir-195(2) | 0.000229 | 0.000819 | −3.58 | 1.10E−06 | 1.00E−05 |
| mir-370(1) | 0.000186 | 0.000046 | 4.08 | 1.99E−06 | 1.74E−05 |
| mir-28(1) | 0.002143 | 0.000645 | 3.32 | 3.40E−06 | 2.75E−05 |
| mir-326(1) | 0.000361 | 0.000097 | 3.73 | 3.41E−06 | 2.75E−05 |
| mir-671(1) | 0.000098 | 0.000026 | 3.77 | 3.66E−06 | 2.84E−05 |
| mir-199a-1(3) | 0.008477 | 0.002391 | 3.55 | 4.87E−06 | 3.65E−05 |
| mir-2355(1) | 0.000095 | 0.000026 | 3.74 | 8.80E−06 | 6.22E−05 |
| mir-505(1) | 0.000236 | 0.000071 | 3.35 | 8.88E−06 | 6.22E−05 |
| mir-3158(1) | 0.000005 | 0.000020 | −4.15 | 1.12E−05 | 7.56E−05 |
| mir-1301(1) | 0.000421 | 0.000137 | 3.08 | 1.81E−05 | 1.18E−04 |
| mir-3688(1) | 0.000002 | 0.000011 | −4.57 | 1.88E−05 | 1.20E−04 |
| mir-223(1) | 0.026945 | 0.008893 | 3.03 | 2.87E−05 | 1.77E−04 |
| mir-625(1) | 0.000684 | 0.000226 | 3.02 | 3.63E−05 | 2.18E−04 |
| mir-199b(1) | 0.003050 | 0.000983 | 3.10 | 4.71E−05 | 2.75E−04 |
| mir-766(1) | 0.000332 | 0.000109 | 3.05 | 4.97E−05 | 2.82E−04 |
| mir-1180(1) | 0.000004 | 0.000017 | −4.05 | 5.72E−05 | 3.16E−04 |
| mir-584(1) | 0.000956 | 0.000339 | 2.82 | 6.04E−05 | 3.25E−04 |
| mir-511-1(2) | 0.000004 | 0.000016 | −3.71 | 8.05E−05 | 4.22E−04 |
| mir-33b(1) | 0.000059 | 0.000013 | 4.67 | 1.06E−04 | 5.35E−04 |
| mir-590(1) | 0.000239 | 0.000084 | 2.86 | 1.07E−04 | 5.35E−04 |
| mir-506(11) | 0.000003 | 0.000013 | −4.30 | 1.14E−04 | 5.55E−04 |
| mir-491(1) | 0.000045 | 0.000014 | 3.25 | 1.41E−04 | 6.67E−04 |
| mir-221(2) | 0.012659 | 0.004877 | 2.60 | 1.43E−04 | 6.67E−04 |
| mir-143(2) | 0.001757 | 0.004630 | −2.63 | 1.79E−04 | 8.18E−04 |
| mir-328(1) | 0.000373 | 0.000136 | 2.74 | 1.93E−04 | 8.63E−04 |
| mir-744(1) | 0.002764 | 0.001075 | 2.57 | 2.95E−04 | 1.29E−03 |
| mir-455(1) | 0.000001 | 0.000004 | −5.27 | 3.16E−04 | 1.36E−03 |
| mir-1249(1) | 0.000010 | 0.000002 | 5.88 | 4.25E−04 | 1.78E−03 |
| mir-29a(4) | 0.004268 | 0.010023 | −2.35 | 6.36E−04 | 2.62E−03 |
| mir-652(1) | 0.002237 | 0.000948 | 2.36 | 6.69E−04 | 2.67E−03 |
| mir-192(4) | 0.000484 | 0.001158 | −2.39 | 6.74E−04 | 2.67E−03 |
| mir-301a(2) | 0.001129 | 0.000469 | 2.41 | 8.28E−04 | 3.22E−03 |
| mir-127(8) | 0.001498 | 0.000610 | 2.46 | 9.07E−04 | 3.46E−03 |
| mir-148a(1) | 0.004812 | 0.011087 | −2.30 | 1.04E−03 | 3.88E−03 |
| mir-551b(1) | 0.000018 | 0.000005 | 3.39 | 1.17E−03 | 4.32E−03 |
| mir-135a-1(3) | 0.012822 | 0.005782 | 2.22 | 1.55E−03 | 5.63E−03 |
| mir-188(8) | 0.000565 | 0.001237 | −2.19 | 1.91E−03 | 6.78E−03 |
| mir-550-1(2) | 0.000044 | 0.000101 | −2.30 | 2.12E−03 | 7.42E−03 |
| mir-3617(1) | 0.000011 | 0.000003 | 3.57 | 2.23E−03 | 7.68E−03 |
| mir-3065(1) | 0.000020 | 0.000007 | 2.82 | 2.88E−03 | 9.74E−03 |
| mir-424(2) | 0.000681 | 0.001443 | −2.12 | 2.93E−03 | 9.76E−03 |
| mir-134(41) | 0.005330 | 0.002473 | 2.16 | 3.03E−03 | 9.95E−03 |
| mir-196a-1(3) | 0.000006 | 0.000016 | −2.51 | 4.03E−03 | 1.30E−02 |
| mir-126(1) | 0.032411 | 0.016053 | 2.02 | 4.95E−03 | 1.55E−02 |
| mir-885(1) | 0.000005 | 0.000014 | −2.66 | 4.95E−03 | 1.55E−02 |
| mir-181a-1(4) | 0.003983 | 0.001963 | 2.03 | 5.16E−03 | 1.59E−02 |
| mir-153-1(2) | 0.000026 | 0.000010 | 2.50 | 5.63E−03 | 1.71E−02 |
| mir-589(1) | 0.000045 | 0.000020 | 2.24 | 6.10E−03 | 1.83E−02 |
| mir-98(13) | 0.097745 | 0.049749 | 1.96 | 6.28E−03 | 1.86E−02 |
| mir-3138(1) | 0.000013 | 0.000005 | 2.71 | 6.98E−03 | 2.04E−02 |
| mir-128-1(2) | 0.001365 | 0.000669 | 2.04 | 7.70E−03 | 2.21E−02 |
| mir-324(1) | 0.000577 | 0.000294 | 1.96 | 7.85E−03 | 2.23E−02 |
| mir-651(1) | 0.000012 | 0.000028 | −2.25 | 8.36E−03 | 2.34E−02 |
| mir-1908(1) | 0.000016 | 0.000006 | 2.53 | 9.46E−03 | 2.61E−02 |
| mir-2110(1) | 0.000058 | 0.000028 | 2.07 | 1.11E−02 | 3.02E−02 |
| mir-331(1) | 0.000133 | 0.000067 | 1.97 | 1.13E−02 | 3.03E−02 |
| mir-1250(1) | 0.000007 | 0.000002 | 3.21 | 1.14E−02 | 3.04E−02 |
| mir-224(2) | 0.000861 | 0.000445 | 1.94 | 1.25E−02 | 3.28E−02 |
| mir-203(1) | 0.000015 | 0.000032 | −2.14 | 1.34E−02 | 3.47E−02 |
| mir-605(1) | 0.000010 | 0.000003 | 2.83 | 1.36E−02 | 3.49E−02 |
| mir-1271(1) | 0.000022 | 0.000011 | 2.10 | 2.10E−02 | 5.28E−02 |
| mir-146a(1) | 0.019114 | 0.010777 | 1.77 | 2.11E−02 | 5.28E−02 |
| let-7i(1) | 0.014473 | 0.008165 | 1.77 | 2.26E−02 | 5.58E−02 |
| mir-151(1) | 0.010711 | 0.006086 | 1.76 | 2.58E−02 | 6.30E−02 |
| mir-769(1) | 0.000132 | 0.000073 | 1.81 | 2.75E−02 | 6.64E−02 |
| mir-1179(1) | 0.000012 | 0.000005 | 2.39 | 2.78E−02 | 6.64E−02 |
| mir-556(1) | 0.000045 | 0.000022 | 1.98 | 2.83E−02 | 6.67E−02 |
| mir-3909(1) | 0.000002 | 0.000005 | −2.35 | 2.96E−02 | 6.86E−02 |
| mir-1296(1) | 0.000011 | 0.000004 | 2.42 | 2.97E−02 | 6.86E−02 |
| mir-574(1) | 0.000343 | 0.000197 | 1.74 | 3.19E−02 | 7.28E−02 |
| mir-2277(1) | 0.000010 | 0.000005 | 2.20 | 3.55E−02 | 8.02E−02 |
| mir-3200(1) | 0.000003 | 0.000006 | −2.20 | 3.74E−02 | 8.36E−02 |
| mir-3074(1) | 0.000013 | 0.000006 | 2.08 | 3.94E−02 | 8.70E−02 |
| mir-32(1) | 0.000286 | 0.000479 | −1.68 | 4.42E−02 | 9.67E−02 |
| mir-498(46) | 0.000008 | 0.000016 | −1.88 | 5.12E−02 | 1.11E−01 |
| mir-190b(1) | 0.000013 | 0.000007 | 1.98 | 5.32E−02 | 1.13E−01 |
| mir-155(1) | 0.000277 | 0.000169 | 1.64 | 5.33E−02 | 1.13E−01 |
| mir-330(1) | 0.000210 | 0.000125 | 1.68 | 5.42E−02 | 1.14E−01 |
| mir-423(1) | 0.008832 | 0.005557 | 1.59 | 6.38E−02 | 1.32E−01 |
| mir-26a-1(2) | 0.054920 | 0.034096 | 1.61 | 6.41E−02 | 1.32E−01 |
| mir-643(1) | 0.000005 | 0.000002 | 2.43 | 6.62E−02 | 1.35E−01 |
| mir-191(2) | 0.024682 | 0.015753 | 1.57 | 7.02E−02 | 1.42E−01 |
| mir-146b(1) | 0.001442 | 0.000919 | 1.57 | 7.14E−02 | 1.43E−01 |
| mir-582(1) | 0.000006 | 0.000012 | −1.89 | 7.49E−02 | 1.48E−01 |
| mir-185(1) | 0.008273 | 0.005289 | 1.56 | 7.57E−02 | 1.49E−01 |
| mir-130a(1) | 0.003800 | 0.002481 | 1.53 | 8.75E−02 | 1.70E−01 |
| mir-7-1(3) | 0.000455 | 0.000684 | −1.50 | 1.09E−01 | 2.08E−01 |
| mir-101-1(2) | 0.003883 | 0.002542 | 1.53 | 1.09E−01 | 2.08E−01 |
| mir-130b(2) | 0.000850 | 0.000571 | 1.49 | 1.13E−01 | 2.14E−01 |
| mir-92b(1) | 0.000049 | 0.000073 | −1.50 | 1.35E−01 | 2.52E−01 |
| mir-3667(1) | 0.000003 | 0.000006 | −1.80 | 1.35E−01 | 2.52E−01 |
| mir-375(1) | 0.000059 | 0.000035 | 1.69 | 1.39E−01 | 2.57E−01 |
| mir-504(1) | 0.000005 | 0.000002 | 2.00 | 1.44E−01 | 2.63E−01 |
| mir-9-1(3) | 0.000030 | 0.000019 | 1.57 | 1.45E−01 | 2.63E−01 |
| mir-760(1) | 0.000047 | 0.000071 | −1.52 | 1.47E−01 | 2.63E−01 |
| mir-339(1) | 0.000826 | 0.000583 | 1.42 | 1.58E−01 | 2.81E−01 |
| mir-196b(1) | 0.000078 | 0.000054 | 1.46 | 1.63E−01 | 2.88E−01 |
| mir-598(1) | 0.000056 | 0.000037 | 1.52 | 1.73E−01 | 3.02E−01 |
| mir-25(3) | 0.015801 | 0.021790 | −1.38 | 1.93E−01 | 3.35E−01 |
| mir-200a(3) | 0.000047 | 0.000066 | −1.39 | 2.19E−01 | 3.76E−01 |
| mir-3187(1) | 0.000007 | 0.000010 | −1.50 | 2.24E−01 | 3.82E−01 |
| mir-340(1) | 0.001590 | 0.001176 | 1.35 | 2.26E−01 | 3.82E−01 |
| mir-3120(1) | 0.000026 | 0.000018 | 1.45 | 2.27E−01 | 3.82E−01 |
| mir-624(1) | 0.000013 | 0.000019 | −1.44 | 2.46E−01 | 4.08E−01 |
| mir-103-1(2) | 0.032402 | 0.024347 | 1.33 | 2.47E−01 | 4.08E−01 |
| mir-23a(6) | 0.047409 | 0.035934 | 1.32 | 2.56E−01 | 4.19E−01 |
| mir-1468(1) | 0.000006 | 0.000004 | 1.61 | 2.79E−01 | 4.53E−01 |
| mir-3176(1) | 0.000003 | 0.000004 | −1.56 | 2.80E−01 | 4.53E−01 |
| mir-21(1) | 0.093327 | 0.071949 | 1.30 | 2.91E−01 | 4.60E−01 |
| mir-204(1) | 0.000018 | 0.000025 | −1.41 | 2.91E−01 | 4.60E−01 |
| mir-570(1) | 0.000008 | 0.000012 | −1.42 | 2.91E−01 | 4.60E−01 |
| mir-2278(1) | 0.000004 | 0.000003 | 1.60 | 2.99E−01 | 4.68E−01 |
| mir-3173(1) | 0.000005 | 0.000007 | −1.43 | 3.03E−01 | 4.72E−01 |
| mir-1255a(1) | 0.000012 | 0.000008 | 1.42 | 3.06E−01 | 4.72E−01 |
| mir-205(1) | 0.000007 | 0.000010 | −1.46 | 3.14E−01 | 4.76E−01 |
| mir-3613(1) | 0.000149 | 0.000196 | −1.31 | 3.15E−01 | 4.76E−01 |
| mir-1284(1) | 0.000004 | 0.000005 | −1.46 | 3.15E−01 | 4.76E−01 |
| mir-122(1) | 0.002261 | 0.001689 | 1.34 | 3.29E−01 | 4.91E−01 |
| mir-873(2) | 0.000004 | 0.000005 | −1.52 | 3.30E−01 | 4.91E−01 |
| mir-3679(1) | 0.000004 | 0.000002 | 1.56 | 3.42E−01 | 5.05E−01 |
| mir-338(1) | 0.000083 | 0.000065 | 1.28 | 3.48E−01 | 5.10E−01 |
| mir-1304(1) | 0.000029 | 0.000022 | 1.31 | 3.55E−01 | 5.17E−01 |
| mir-449a(3) | 0.000003 | 0.000004 | −1.43 | 3.72E−01 | 5.39E−01 |
| mir-140(1) | 0.002949 | 0.003708 | −1.26 | 3.79E−01 | 5.45E−01 |
| mir-99b(3) | 0.003268 | 0.002637 | 1.24 | 3.84E−01 | 5.46E−01 |
| mir-3942(1) | 0.000004 | 0.000002 | 1.50 | 3.85E−01 | 5.46E−01 |
| mir-1256(1) | 0.000011 | 0.000008 | 1.33 | 3.97E−01 | 5.60E−01 |
| mir-129-1(2) | 0.000002 | 0.000003 | −1.47 | 4.01E−01 | 5.60E−01 |
| mir-3611(1) | 0.000007 | 0.000005 | 1.39 | 4.03E−01 | 5.60E−01 |
| mir-335(1) | 0.001393 | 0.001699 | −1.22 | 4.15E−01 | 5.73E−01 |
| mir-1270-1(1) | 0.000004 | 0.000006 | −1.35 | 4.29E−01 | 5.80E−01 |
| mir-1270-2(1) | 0.000004 | 0.000006 | −1.35 | 4.29E−01 | 5.80E−01 |
| mir-26b(1) | 0.027185 | 0.022306 | 1.22 | 4.30E−01 | 5.80E−01 |
| mir-3177(1) | 0.000011 | 0.000009 | 1.30 | 4.31E−01 | 5.80E−01 |
| mir-874(1) | 0.000047 | 0.000059 | −1.25 | 4.35E−01 | 5.81E−01 |
| mir-345(1) | 0.000147 | 0.000120 | 1.22 | 4.53E−01 | 6.03E−01 |
| mir-141(2) | 0.000132 | 0.000160 | −1.21 | 4.70E−01 | 6.20E−01 |
| mir-579(1) | 0.000005 | 0.000006 | −1.29 | 5.01E−01 | 6.58E−01 |
| mir-22(1) | 0.038742 | 0.045618 | −1.18 | 5.11E−01 | 6.63E−01 |
| mir-3194(1) | 0.000004 | 0.000005 | −1.30 | 5.14E−01 | 6.63E−01 |
| mir-3140(1) | 0.000002 | 0.000003 | −1.34 | 5.17E−01 | 6.63E−01 |
| mir-186(1) | 0.004706 | 0.005530 | −1.18 | 5.18E−01 | 6.63E−01 |
| mir-197(1) | 0.000594 | 0.000504 | 1.18 | 5.27E−01 | 6.71E−01 |
| mir-3143(1) | 0.000006 | 0.000007 | −1.25 | 5.35E−01 | 6.73E−01 |
| mir-139(1) | 0.000912 | 0.000781 | 1.17 | 5.37E−01 | 6.73E−01 |
| mir-342(1) | 0.000806 | 0.000940 | −1.17 | 5.38E−01 | 6.73E−01 |
| mir-148b(1) | 0.005947 | 0.006898 | −1.16 | 5.67E−01 | 6.99E−01 |
| mir-30b(2) | 0.013482 | 0.011721 | 1.15 | 5.68E−01 | 6.99E−01 |
| mir-483(1) | 0.000014 | 0.000017 | −1.21 | 5.70E−01 | 6.99E−01 |
| mir-210(1) | 0.000243 | 0.000280 | −1.15 | 5.83E−01 | 7.08E−01 |
| mir-576(1) | 0.000057 | 0.000066 | −1.16 | 5.83E−01 | 7.08E−01 |
| mir-34a(1) | 0.000045 | 0.000052 | −1.17 | 5.93E−01 | 7.15E−01 |
| mir-3124(1) | 0.000004 | 0.000003 | 1.22 | 6.53E−01 | 7.84E−01 |
| mir-641(1) | 0.000015 | 0.000013 | 1.15 | 6.68E−01 | 7.92E−01 |
| mir-219-1(2) | 0.000004 | 0.000004 | 1.19 | 6.70E−01 | 7.92E−01 |
| mir-150(1) | 0.000595 | 0.000667 | −1.12 | 6.71E−01 | 7.92E−01 |
| mir-942(1) | 0.000049 | 0.000043 | 1.12 | 6.79E−01 | 7.97E−01 |
| mir-361(1) | 0.001007 | 0.001118 | −1.11 | 6.91E−01 | 8.06E−01 |
| mir-628(1) | 0.000089 | 0.000080 | 1.11 | 6.97E−01 | 8.09E−01 |
| mir-3127(1) | 0.000004 | 0.000003 | 1.17 | 7.19E−01 | 8.24E−01 |
| mir-450a-1(4) | 0.000081 | 0.000089 | −1.10 | 7.20E−01 | 8.24E−01 |
| mir-1287(1) | 0.000012 | 0.000010 | 1.13 | 7.22E−01 | 8.24E−01 |
| mir-132(2) | 0.000082 | 0.000090 | −1.09 | 7.34E−01 | 8.33E−01 |
| mir-152(1) | 0.000948 | 0.000876 | 1.08 | 7.56E−01 | 8.53E−01 |
| mir-3115(1) | 0.000003 | 0.000002 | 1.15 | 7.69E−01 | 8.62E−01 |
| mir-374a(4) | 0.004477 | 0.004160 | 1.08 | 7.72E−01 | 8.62E−01 |
| mir-142(1) | 0.016060 | 0.015005 | 1.07 | 7.81E−01 | 8.66E−01 |
| mir-1306(1) | 0.000063 | 0.000068 | −1.07 | 7.90E−01 | 8.66E−01 |
| mir-616(1) | 0.000008 | 0.000007 | 1.10 | 7.92E−01 | 8.66E−01 |
| mir-17(12) | 0.048059 | 0.045166 | 1.06 | 8.01E−01 | 8.66E−01 |
| mir-107(1) | 0.002357 | 0.002217 | 1.06 | 8.04E−01 | 8.66E−01 |
| mir-3605(1) | 0.000007 | 0.000006 | 1.10 | 8.08E−01 | 8.66E−01 |
| mir-138-1(2) | 0.000003 | 0.000004 | −1.11 | 8.08E−01 | 8.66E−01 |
| mir-3150(1) | 0.000002 | 0.000002 | 1.13 | 8.09E−01 | 8.66E−01 |
| mir-296(1) | 0.000007 | 0.000007 | 1.09 | 8.25E−01 | 8.78E−01 |
| mir-2276(1) | 0.000003 | 0.000003 | 1.09 | 8.30E−01 | 8.78E−01 |
| mir-629(1) | 0.000158 | 0.000149 | 1.06 | 8.32E−01 | 8.78E−01 |
| mir-1294(1) | 0.000009 | 0.000009 | 1.07 | 8.45E−01 | 8.88E−01 |
| mir-490(1) | 0.000005 | 0.000005 | −1.07 | 8.88E−01 | 9.26E−01 |
| mir-627(1) | 0.000036 | 0.000035 | 1.04 | 8.91E−01 | 9.26E−01 |
| mir-320(1) | 0.012279 | 0.011952 | 1.03 | 9.12E−01 | 9.44E−01 |
| mir-3912(1) | 0.000006 | 0.000006 | −1.04 | 9.18E−01 | 9.45E−01 |
| mir-4326(1) | 0.000007 | 0.000007 | −1.04 | 9.23E−01 | 9.45E−01 |
| mir-30a(4) | 0.014321 | 0.014643 | −1.02 | 9.28E−01 | 9.46E−01 |
| mir-484(1) | 0.002768 | 0.002716 | 1.02 | 9.38E−01 | 9.50E−01 |
| mir-3157(1) | 0.000005 | 0.000005 | −1.03 | 9.41E−01 | 9.50E−01 |
| mir-3940(1) | 0.000006 | 0.000007 | −1.02 | 9.48E−01 | 9.51E−01 |
| mir-3928(1) | 0.000011 | 0.000011 | 1.02 | 9.51E−01 | 9.51E−01 |

Table 6 is analysis of miRNA cistron abundance changes comparing plasma from patients with advanced heart failure at LVAD explantation to plasma from healthy volunteers (NF). The normalized read frequency is represented as a fraction. The false discovery rate (FDR) was calculated by the method of Benjamini and Hochberg.

TABLE 6
Differences in the plasma levels in advanced heart failure
(LVAD explantation) compared to healthy controls

| | Normalized Read | | | | |
|---|---|---|---|---|---|
| | Frequency | | | | |
| | LVAD | | | | |
| miRNA | Explanta- | Healthy | Fold | | |
| Cistron | tion | Controls | Change | P value | FDR |
| mir-208a(1) | 0.000084 | 0.000002 | 54.03 | 2.31E−18 | 5.53E−16 |
| mir-208b(1) | 0.000138 | 0.000002 | 89.08 | 5.89E−18 | 7.03E−16 |
| mir-1180(1) | 0.000066 | 0.000003 | 20.35 | 3.10E−17 | 2.47E−15 |
| mir-1277(1) | 0.000009 | 0.000481 | −55.62 | 9.55E−15 | 4.73E−13 |
| mir-378(1) | 0.008782 | 0.000669 | 13.13 | 9.90E−15 | 4.73E−13 |
| mir-126(1) | 0.005752 | 0.051673 | −8.98 | 1.59E−13 | 6.31E−12 |
| mir-133b(2) | 0.000580 | 0.000017 | 34.58 | 3.62E−11 | 1.24E−09 |
| mir-33a(1) | 0.000026 | 0.000357 | −13.98 | 7.51E−11 | 2.24E−09 |
| mir-148a(1) | 0.037109 | 0.005122 | 7.25 | 1.19E−10 | 3.16E−09 |
| mir-551b(1) | 0.000000 | 0.000026 | −95.65 | 2.96E−10 | 7.08E−09 |
| mir-135a-1(3) | 0.002904 | 0.016871 | −5.81 | 5.16E−10 | 1.12E−08 |
| mir-652(1) | 0.000558 | 0.002893 | −5.19 | 9.40E−10 | 1.87E−08 |
| mir-506(11) | 0.000176 | 0.000002 | 82.53 | 1.12E−09 | 2.05E−08 |
| mir-3158(1) | 0.000037 | 0.000004 | 8.64 | 3.49E−09 | 5.95E−08 |
| mir-326(1) | 0.000023 | 0.000345 | −14.99 | 4.87E−09 | 7.42E−08 |
| mir-193a(4) | 0.000954 | 0.000128 | 7.44 | 4.97E−09 | 7.42E−08 |
| mir-3065(1) | 0.000001 | 0.000026 | −37.62 | 7.05E−09 | 9.59E−08 |
| mir-196b(1) | 0.000017 | 0.000140 | −8.41 | 7.22E−09 | 9.59E−08 |
| mir-499(1) | 0.000114 | 0.000012 | 9.24 | 7.90E−09 | 9.94E−08 |
| mir-221(2) | 0.004268 | 0.017813 | −4.17 | 9.87E−09 | 1.18E−07 |
| mir-625(1) | 0.000076 | 0.000605 | −7.91 | 4.84E−08 | 5.50E−07 |
| mir-218-1(3) | 0.000057 | 0.000003 | 18.08 | 9.38E−08 | 1.02E−06 |
| mir-140(1) | 0.014419 | 0.003653 | 3.95 | 3.01E−07 | 3.13E−06 |
| mir-190a(1) | 0.000005 | 0.000091 | −19.31 | 3.20E−07 | 3.19E−06 |
| mir-370(1) | 0.000032 | 0.000332 | −10.39 | 3.83E−07 | 3.67E−06 |
| mir-216a(3) | 0.000024 | 0.000001 | 17.57 | 6.79E−07 | 6.24E−06 |
| mir-223(1) | 0.005772 | 0.034159 | −5.92 | 8.48E−07 | 7.50E−06 |
| mir-590(1) | 0.000035 | 0.000353 | −10.21 | 1.05E−06 | 8.94E−06 |
| mir-25(3) | 0.062365 | 0.020320 | 3.07 | 1.92E−06 | 1.58E−05 |
| mir-96(3) | 0.002388 | 0.000505 | 4.73 | 2.27E−06 | 1.77E−05 |
| mir-374a(4) | 0.001188 | 0.005337 | −4.49 | 2.30E−06 | 1.77E−05 |
| mir-143(2) | 0.012469 | 0.002849 | 4.38 | 2.98E−06 | 2.17E−05 |
| mir-3157(1) | 0.000011 | 0.000002 | 6.09 | 3.00E−06 | 2.17E−05 |
| mir-1301(1) | 0.000117 | 0.000470 | −4.00 | 3.30E−06 | 2.32E−05 |
| mir-486(1) | 0.086970 | 0.017249 | 5.04 | 3.64E−06 | 2.48E−05 |
| mir-15a(4) | 0.094869 | 0.022984 | 4.13 | 3.97E−06 | 2.63E−05 |
| mir-328(1) | 0.000115 | 0.000683 | −5.96 | 4.17E−06 | 2.69E−05 |
| mir-491(1) | 0.000005 | 0.000051 | −9.79 | 6.98E−06 | 4.39E−05 |
| mir-188(8) | 0.002129 | 0.000670 | 3.18 | 8.71E−06 | 5.33E−05 |
| mir-873(2) | 0.000016 | 0.000003 | 6.43 | 9.62E−06 | 5.75E−05 |
| mir-1249(1) | 0.000002 | 0.000014 | −8.20 | 1.36E−05 | 7.93E−05 |
| mir-488(1) | 0.000013 | 0.000001 | 25.06 | 1.44E−05 | 8.17E−05 |
| mir-584(1) | 0.000480 | 0.002483 | −5.17 | 1.87E−05 | 1.04E−04 |
| mir-675(1) | 0.000012 | 0.000001 | 13.34 | 1.92E−05 | 1.04E−04 |
| mir-671(1) | 0.000030 | 0.000143 | −4.68 | 3.31E−05 | 1.76E−04 |
| mir-181c(2) | 0.000198 | 0.000651 | −3.29 | 3.61E−05 | 1.88E−04 |
| mir-556(1) | 0.000006 | 0.000046 | −7.23 | 3.94E−05 | 2.01E−04 |
| mir-210(1) | 0.000486 | 0.000150 | 3.25 | 5.56E−05 | 2.77E−04 |
| mir-10a(1) | 0.004335 | 0.001088 | 3.99 | 9.76E−05 | 4.76E−04 |
| mir-134(41) | 0.002145 | 0.008940 | −4.17 | 1.06E−04 | 5.05E−04 |
| mir-3124(1) | 0.000016 | 0.000003 | 4.96 | 1.13E−04 | 5.28E−04 |
| mir-3691(1) | 0.000006 | 0.000001 | 9.49 | 1.21E−04 | 5.57E−04 |
| mir-2277(1) | 0.000001 | 0.000011 | −14.75 | 1.26E−04 | 5.68E−04 |
| mir-301a(2) | 0.000254 | 0.001138 | −4.47 | 1.73E−04 | 7.54E−04 |
| mir-195(2) | 0.000594 | 0.000180 | 3.30 | 1.74E−04 | 7.54E−04 |
| mir-155(1) | 0.000152 | 0.000449 | −2.94 | 1.98E−04 | 8.34E−04 |
| mir-641(1) | 0.000004 | 0.000025 | −6.59 | 1.99E−04 | 8.34E−04 |
| mir-1179(1) | 0.000001 | 0.000013 | −9.16 | 2.05E−04 | 8.47E−04 |
| mir-150(1) | 0.000401 | 0.001640 | −4.09 | 2.28E−04 | 9.23E−04 |
| mir-1247(1) | 0.000006 | 0.000001 | 9.47 | 2.40E−04 | 9.56E−04 |
| mir-146b(1) | 0.000565 | 0.001518 | −2.69 | 2.62E−04 | 1.03E−03 |
| mir-1250(1) | 0.000002 | 0.000018 | −9.46 | 2.79E−04 | 1.07E−03 |
| mir-580(1) | 0.000000 | 0.000004 | −48.14 | 2.91E−04 | 1.10E−03 |
| mir-31(1) | 0.000004 | 0.000000 | 7.44 | 2.95E−04 | 1.10E−03 |
| mir-744(1) | 0.001347 | 0.003692 | −2.74 | 2.99E−04 | 1.10E−03 |
| mir-1-1(4) | 0.001648 | 0.000525 | 3.14 | 3.15E−04 | 1.14E−03 |
| mir-184(1) | 0.000008 | 0.000001 | 11.17 | 3.48E−04 | 1.24E−03 |
| mir-576(1) | 0.000140 | 0.000044 | 3.15 | 3.99E−04 | 1.40E−03 |
| mir-127(8) | 0.000917 | 0.003256 | −3.55 | 5.30E−04 | 1.84E−03 |
| mir-3688(1) | 0.000018 | 0.000004 | 5.20 | 5.69E−04 | 1.94E−03 |
| mir-10b(1) | 0.004142 | 0.000912 | 4.54 | 6.16E−04 | 2.07E−03 |
| mir-1307(1) | 0.000365 | 0.001086 | −2.98 | 8.63E−04 | 2.87E−03 |
| mir-552(1) | 0.000001 | 0.000005 | −9.43 | 9.37E−04 | 3.07E−03 |
| mir-28(1) | 0.001564 | 0.003863 | −2.47 | 1.09E−03 | 3.51E−03 |
| mir-26a-1(2) | 0.022141 | 0.059280 | −2.68 | 1.23E−03 | 3.91E−03 |
| mir-449a(3) | 0.000000 | 0.000005 | −14.05 | 1.38E−03 | 4.35E−03 |
| mir-23a(6) | 0.025615 | 0.054213 | −2.12 | 1.53E−03 | 4.74E−03 |
| mir-98(13) | 0.050631 | 0.104892 | −2.07 | 1.64E−03 | 5.04E−03 |
| mir-95(1) | 0.000038 | 0.000009 | 4.14 | 1.75E−03 | 5.29E−03 |
| mir-375(1) | 0.000062 | 0.000466 | −7.52 | 2.26E−03 | 6.74E−03 |
| mir-2355(1) | 0.000029 | 0.000114 | −3.91 | 2.38E−03 | 7.04E−03 |
| mir-610(1) | 0.000005 | 0.000001 | 3.90 | 2.51E−03 | 7.33E−03 |
| mir-550-1(2) | 0.000085 | 0.000036 | 2.33 | 2.95E−03 | 8.49E−03 |
| let-7i(1) | 0.009019 | 0.018597 | −2.06 | 3.04E−03 | 8.64E−03 |
| mir-3194(1) | 0.000001 | 0.000004 | −6.99 | 3.11E−03 | 8.68E−03 |
| mir-130a(1) | 0.001612 | 0.003928 | −2.44 | 3.12E−03 | 8.68E−03 |
| mir-490(1) | 0.000012 | 0.000003 | 4.36 | 3.53E−03 | 9.69E−03 |
| mir-330(1) | 0.000053 | 0.000172 | −3.22 | 4.06E−03 | 1.10E−02 |
| mir-324(1) | 0.000304 | 0.000615 | −2.03 | 4.50E−03 | 1.21E−02 |
| mir-511-1(2) | 0.000026 | 0.000008 | 3.30 | 4.56E−03 | 1.21E−02 |
| mir-1306(1) | 0.000124 | 0.000053 | 2.33 | 4.67E−03 | 1.23E−02 |
| mir-455(1) | 0.000006 | 0.000001 | 4.27 | 5.30E−03 | 1.38E−02 |
| mir-1271(1) | 0.000009 | 0.000030 | −3.51 | 5.71E−03 | 1.46E−02 |
| mir-3934(1) | 0.000004 | 0.000001 | 7.88 | 5.75E−03 | 1.46E−02 |
| mir-320(1) | 0.014764 | 0.008000 | 1.85 | 6.54E−03 | 1.65E−02 |
| mir-7-1(3) | 0.000624 | 0.000279 | 2.24 | 7.46E−03 | 1.86E−02 |
| mir-34b(2) | 0.000029 | 0.000007 | 4.31 | 8.02E−03 | 1.98E−02 |
| mir-187(1) | 0.000007 | 0.000002 | 3.61 | 8.68E−03 | 2.12E−02 |
| mir-185(1) | 0.003222 | 0.006338 | −1.97 | 9.43E−03 | 2.28E−02 |
| mir-339(1) | 0.000444 | 0.000948 | −2.14 | 9.97E−03 | 2.38E−02 |
| mir-505(1) | 0.000087 | 0.000230 | −2.63 | 1.03E−02 | 2.43E−02 |
| mir-887(1) | 0.000005 | 0.000001 | 4.27 | 1.10E−02 | 2.55E−02 |
| mir-1270-1(1) | 0.000007 | 0.000003 | 2.58 | 1.11E−02 | 2.55E−02 |
| mir-1270-2(1) | 0.000007 | 0.000003 | 2.58 | 1.11E−02 | 2.55E−02 |
| mir-142(1) | 0.009341 | 0.019626 | −2.10 | 1.20E−02 | 2.72E−02 |
| mir-643(1) | 0.000002 | 0.000006 | −3.24 | 1.21E−02 | 2.74E−02 |
| mir-191(2) | 0.012769 | 0.025093 | −1.97 | 1.25E−02 | 2.80E−02 |
| mir-489(2) | 0.000006 | 0.000001 | 4.28 | 1.29E−02 | 2.86E−02 |
| mir-146a(1) | 0.011734 | 0.021528 | −1.83 | 1.31E−02 | 2.88E−02 |
| mir-766(1) | 0.000089 | 0.000292 | −3.30 | 1.43E−02 | 3.10E−02 |
| mir-3187(1) | 0.000020 | 0.000009 | 2.29 | 1.58E−02 | 3.40E−02 |
| mir-3942(1) | 0.000006 | 0.000002 | 2.84 | 1.71E−02 | 3.65E−02 |
| mir-424(2) | 0.000951 | 0.000477 | 2.00 | 1.74E−02 | 3.65E−02 |
| mir-629(1) | 0.000430 | 0.000182 | 2.36 | 1.74E−02 | 3.65E−02 |
| mir-582(1) | 0.000043 | 0.000014 | 3.06 | 1.81E−02 | 3.76E−02 |
| mir-202(1) | 0.000004 | 0.000001 | 4.52 | 2.03E−02 | 4.17E−02 |
| mir-3177(1) | 0.000008 | 0.000022 | −2.88 | 2.19E−02 | 4.47E−02 |
| mir-1256(1) | 0.000002 | 0.000011 | −4.32 | 2.22E−02 | 4.49E−02 |
| mir-3617(1) | 0.000004 | 0.000015 | −3.85 | 2.23E−02 | 4.49E−02 |
| mir-605(1) | 0.000002 | 0.000008 | −5.29 | 2.45E−02 | 4.87E−02 |
| mir-219-1(2) | 0.000006 | 0.000002 | 2.64 | 2.59E−02 | 5.12E−02 |
| mir-3614(1) | 0.000003 | 0.000001 | 4.09 | 2.84E−02 | 5.56E−02 |
| mir-3613(1) | 0.000292 | 0.000127 | 2.30 | 3.27E−02 | 6.35E−02 |
| mir-197(1) | 0.000907 | 0.000472 | 1.92 | 3.48E−02 | 6.71E−02 |
| mir-22(1) | 0.044235 | 0.023820 | 1.86 | 3.88E−02 | 7.42E−02 |
| mir-484(1) | 0.002394 | 0.004092 | −1.71 | 4.21E−02 | 7.99E−02 |
| mir-130b(2) | 0.000426 | 0.000716 | −1.68 | 4.46E−02 | 8.40E−02 |
| mir-107(1) | 0.003368 | 0.001689 | 1.99 | 4.87E−02 | 9.09E−02 |
| mir-628(1) | 0.000040 | 0.000083 | −2.07 | 5.44E−02 | 1.01E−01 |
| mir-504(1) | 0.000002 | 0.000004 | −2.57 | 5.52E−02 | 1.01E−01 |
| mir-30b(2) | 0.031055 | 0.019632 | 1.58 | 5.54E−02 | 1.01E−01 |
| mir-331(1) | 0.000084 | 0.000149 | −1.78 | 5.79E−02 | 1.05E−01 |
| mir-192(4) | 0.002296 | 0.001213 | 1.89 | 6.28E−02 | 1.13E−01 |
| mir-204(1) | 0.000031 | 0.000016 | 1.96 | 6.44E−02 | 1.15E−01 |
| mir-769(1) | 0.000150 | 0.000290 | −1.93 | 6.48E−02 | 1.15E−01 |
| mir-200a(3) | 0.000067 | 0.000151 | −2.27 | 7.31E−02 | 1.28E−01 |
| mir-9-1(3) | 0.000017 | 0.000040 | −2.36 | 7.32E−02 | 1.28E−01 |
| mir-3136(1) | 0.000001 | 0.000003 | −3.81 | 7.77E−02 | 1.35E−01 |
| mir-3909(1) | 0.000004 | 0.000002 | 2.35 | 7.85E−02 | 1.35E−01 |
| mir-3140(1) | 0.000003 | 0.000001 | 2.61 | 7.90E−02 | 1.35E−01 |
| mir-342(1) | 0.000705 | 0.001141 | −1.62 | 8.59E−02 | 1.46E−01 |
| mir-124-1(3) | 0.000009 | 0.000003 | 2.66 | 8.66E−02 | 1.46E−01 |
| mir-3130(1) | 0.000007 | 0.000003 | 2.53 | 8.82E−02 | 1.47E−01 |
| mir-3174(1) | 0.000001 | 0.000002 | −2.39 | 9.14E−02 | 1.51E−01 |
| mir-153-1(2) | 0.000009 | 0.000021 | −2.40 | 9.17E−02 | 1.51E−01 |
| mir-2116(1) | 0.000007 | 0.000003 | 2.61 | 9.44E−02 | 1.54E−01 |
| mir-2276(1) | 0.000003 | 0.000002 | 1.91 | 9.56E−02 | 1.55E−01 |
| mir-3679(1) | 0.000001 | 0.000003 | −4.45 | 9.78E−02 | 1.58E−01 |
| mir-3198(1) | 0.000001 | 0.000003 | −5.66 | 1.01E−01 | 1.61E−01 |
| mir-3940(1) | 0.000009 | 0.000005 | 1.92 | 1.09E−01 | 1.73E−01 |
| mir-498(46) | 0.000006 | 0.000013 | −2.30 | 1.10E−01 | 1.73E−01 |
| mir-574(1) | 0.000273 | 0.000408 | −1.50 | 1.10E−01 | 1.73E−01 |
| mir-186(1) | 0.002633 | 0.004201 | −1.60 | 1.11E−01 | 1.74E−01 |
| mir-3074(1) | 0.000005 | 0.000012 | −2.31 | 1.14E−01 | 1.78E−01 |
| mir-874(1) | 0.000024 | 0.000046 | −1.95 | 1.15E−01 | 1.78E−01 |
| mir-296(1) | 0.000009 | 0.000006 | 1.69 | 1.17E−01 | 1.79E−01 |
| mir-199a-1(3) | 0.006548 | 0.010838 | −1.66 | 1.29E−01 | 1.96E−01 |
| mir-483(1) | 0.000015 | 0.000031 | −2.10 | 1.31E−01 | 1.98E−01 |
| mir-101-1(2) | 0.013288 | 0.007920 | 1.68 | 1.32E−01 | 1.98E−01 |
| mir-128-1(2) | 0.000900 | 0.001399 | −1.55 | 1.33E−01 | 1.98E−01 |
| mir-579(1) | 0.000004 | 0.000007 | −1.89 | 1.37E−01 | 2.03E−01 |
| mir-1284(1) | 0.000003 | 0.000006 | −2.58 | 1.39E−01 | 2.05E−01 |
| mir-3120(1) | 0.000011 | 0.000021 | −1.88 | 1.40E−01 | 2.05E−01 |
| mir-139(1) | 0.000546 | 0.000909 | −1.67 | 1.40E−01 | 2.05E−01 |
| mir-190b(1) | 0.000008 | 0.000014 | −1.64 | 1.42E−01 | 2.06E−01 |
| mir-3611(1) | 0.000010 | 0.000006 | 1.82 | 1.49E−01 | 2.14E−01 |
| mir-1908(1) | 0.000009 | 0.000005 | 1.81 | 1.50E−01 | 2.15E−01 |
| mir-29a(4) | 0.007539 | 0.005255 | 1.43 | 1.61E−01 | 2.28E−01 |
| mir-624(1) | 0.000018 | 0.000010 | 1.74 | 1.67E−01 | 2.36E−01 |
| mir-423(1) | 0.007126 | 0.009939 | −1.39 | 1.71E−01 | 2.41E−01 |
| mir-642(1) | 0.000002 | 0.000004 | −2.26 | 1.72E−01 | 2.41E−01 |
| mir-30a(4) | 0.028435 | 0.019097 | 1.49 | 1.73E−01 | 2.41E−01 |
| mir-1294(1) | 0.000009 | 0.000005 | 1.89 | 1.94E−01 | 2.68E−01 |
| mir-3605(1) | 0.000014 | 0.000009 | 1.60 | 2.02E−01 | 2.77E−01 |
| mir-144(2) | 0.100463 | 0.062630 | 1.60 | 2.04E−01 | 2.79E−01 |
| mir-1296(1) | 0.000007 | 0.000016 | −2.25 | 2.32E−01 | 3.15E−01 |
| mir-99b(3) | 0.003458 | 0.004763 | −1.38 | 2.35E−01 | 3.17E−01 |
| mir-3143(1) | 0.000003 | 0.000005 | −1.66 | 2.36E−01 | 3.17E−01 |
| mir-26b(1) | 0.014909 | 0.021165 | −1.42 | 2.46E−01 | 3.28E−01 |
| mir-2110(1) | 0.000058 | 0.000041 | 1.41 | 2.47E−01 | 3.28E−01 |
| mir-338(1) | 0.000073 | 0.000109 | −1.49 | 2.67E−01 | 3.53E−01 |
| mir-92b(1) | 0.000145 | 0.000096 | 1.50 | 2.75E−01 | 3.61E−01 |
| mir-152(1) | 0.001302 | 0.001011 | 1.29 | 2.89E−01 | 3.77E−01 |
| mir-708(1) | 0.000007 | 0.000004 | 1.62 | 2.94E−01 | 3.81E−01 |
| mir-224(2) | 0.000572 | 0.000839 | −1.47 | 2.97E−01 | 3.84E−01 |
| mir-1255a(1) | 0.000004 | 0.000009 | −1.95 | 2.99E−01 | 3.84E−01 |
| mir-103-1(2) | 0.017907 | 0.024868 | −1.39 | 3.09E−01 | 3.95E−01 |
| mir-651(1) | 0.000016 | 0.000025 | −1.54 | 3.14E−01 | 3.99E−01 |
| mir-32(1) | 0.000280 | 0.000413 | −1.47 | 3.16E−01 | 4.00E−01 |
| mir-361(1) | 0.001724 | 0.001276 | 1.35 | 3.18E−01 | 4.00E−01 |
| mir-2278(1) | 0.000002 | 0.000003 | −1.74 | 3.21E−01 | 4.02E−01 |
| mir-942(1) | 0.000069 | 0.000053 | 1.31 | 3.31E−01 | 4.12E−01 |
| mir-33b(1) | 0.000023 | 0.000015 | 1.55 | 3.49E−01 | 4.30E−01 |
| mir-3138(1) | 0.000006 | 0.000010 | −1.55 | 3.49E−01 | 4.30E−01 |
| mir-138-1(2) | 0.000002 | 0.000005 | −1.91 | 3.57E−01 | 4.37E−01 |
| mir-181a-1(4) | 0.006270 | 0.007952 | −1.27 | 3.61E−01 | 4.40E−01 |
| mir-122(1) | 0.006016 | 0.004059 | 1.48 | 3.73E−01 | 4.53E−01 |
| mir-3928(1) | 0.000009 | 0.000013 | −1.49 | 3.82E−01 | 4.62E−01 |
| mir-3667(1) | 0.000005 | 0.000003 | 1.60 | 3.85E−01 | 4.62E−01 |
| mir-196a-1(3) | 0.000009 | 0.000015 | −1.61 | 3.89E−01 | 4.64E−01 |
| mir-3677(1) | 0.000001 | 0.000003 | −2.01 | 3.91E−01 | 4.64E−01 |
| mir-627(1) | 0.000034 | 0.000044 | −1.29 | 4.11E−01 | 4.86E−01 |
| mir-141(2) | 0.000227 | 0.000317 | −1.40 | 4.24E−01 | 5.00E−01 |
| mir-132(2) | 0.000146 | 0.000184 | −1.26 | 4.27E−01 | 5.00E−01 |
| mir-1468(1) | 0.000007 | 0.000011 | −1.62 | 4.29E−01 | 5.00E−01 |
| mir-149(1) | 0.000002 | 0.000002 | 1.58 | 4.36E−01 | 5.06E−01 |
| mir-3155(1) | 0.000001 | 0.000002 | −1.65 | 4.54E−01 | 5.24E−01 |
| mir-340(1) | 0.001459 | 0.001860 | −1.28 | 4.63E−01 | 5.32E−01 |
| mir-34a(1) | 0.000046 | 0.000063 | −1.38 | 4.89E−01 | 5.59E−01 |
| mir-760(1) | 0.000065 | 0.000097 | −1.49 | 5.15E−01 | 5.86E−01 |
| mir-937(1) | 0.000003 | 0.000002 | 1.56 | 5.31E−01 | 6.02E−01 |
| mir-3127(1) | 0.000003 | 0.000005 | −1.42 | 5.66E−01 | 6.39E−01 |
| mir-450a-1(4) | 0.000151 | 0.000127 | 1.19 | 5.70E−01 | 6.39E−01 |
| mir-3200(1) | 0.000006 | 0.000004 | 1.41 | 5.73E−01 | 6.40E−01 |
| mir-1287(1) | 0.000021 | 0.000017 | 1.21 | 5.85E−01 | 6.50E−01 |
| mir-589(1) | 0.000051 | 0.000062 | −1.22 | 5.87E−01 | 6.50E−01 |
| mir-199b(1) | 0.003040 | 0.003596 | −1.18 | 6.37E−01 | 7.01E−01 |
| mir-147(1) | 0.000005 | 0.000004 | 1.39 | 6.55E−01 | 7.18E−01 |
| mir-3150(1) | 0.000003 | 0.000002 | 1.29 | 6.63E−01 | 7.24E−01 |
| mir-3176(1) | 0.000002 | 0.000002 | −1.08 | 6.85E−01 | 7.44E−01 |
| mir-570(1) | 0.000005 | 0.000006 | −1.20 | 7.00E−01 | 7.57E−01 |
| mir-1537(1) | 0.000002 | 0.000003 | −1.43 | 7.20E−01 | 7.76E−01 |
| mir-4326(1) | 0.000014 | 0.000012 | 1.18 | 7.28E−01 | 7.80E−01 |
| mir-598(1) | 0.000107 | 0.000096 | 1.11 | 7.52E−01 | 8.02E−01 |
| mir-3173(1) | 0.000008 | 0.000007 | 1.15 | 7.56E−01 | 8.03E−01 |
| mir-335(1) | 0.001328 | 0.001250 | 1.06 | 8.07E−01 | 8.53E−01 |
| mir-885(1) | 0.000020 | 0.000017 | 1.12 | 8.29E−01 | 8.72E−01 |
| mir-17(12) | 0.065025 | 0.062610 | 1.04 | 8.72E−01 | 9.14E−01 |
| mir-129-1(2) | 0.000005 | 0.000005 | −1.13 | 8.83E−01 | 9.22E−01 |
| mir-205(1) | 0.000024 | 0.000023 | 1.04 | 8.97E−01 | 9.33E−01 |
| mir-616(1) | 0.000006 | 0.000006 | −1.03 | 9.29E−01 | 9.58E−01 |
| mir-203(1) | 0.000093 | 0.000091 | 1.02 | 9.30E−01 | 9.58E−01 |
| mir-345(1) | 0.000140 | 0.000144 | −1.03 | 9.41E−01 | 9.64E−01 |
| mir-3115(1) | 0.000003 | 0.000003 | 1.10 | 9.47E−01 | 9.64E−01 |
| mir-21(1) | 0.115957 | 0.118284 | −1.02 | 9.48E−01 | 9.64E−01 |
| mir-3912(1) | 0.000005 | 0.000005 | 1.02 | 9.66E−01 | 9.76E−01 |
| mir-151(1) | 0.015384 | 0.015597 | −1.01 | 9.68E−01 | 9.76E−01 |
| mir-148b(1) | 0.007045 | 0.007099 | −1.01 | 9.98E−01 | 1.00E+00 |
| mir-1304(1) | 0.000042 | 0.000042 | 1.00 | 1.00E+00 | 1.00E+00 |

In stable HF patients, the myomir levels were nearly comparable to NF, and the biggest differences noted were a 5.4-fold increase for mir-375(1) and a 4.5-fold drop for mir-203(1). Furthermore, there were some concordant changes in both stable and advanced HF compared to NF: mir-210(1) was 2.2- and 1.9-fold higher in advanced HF and in stable HF, respectively. mir-1908(1) was 2.0- and 2.1-fold, and mir-1180(1) 4.5- and 4.0-fold higher in patients with advanced HF and in patients with stable HF, respectively (Tables 3-7).

Table 7 is an analysis of miRNA cistron abundance changes comparing plasma from patients with stable heart failure (stable HF) to plasma from healthy volunteers (NF). The normalized read frequency is represented as a fraction. The false discovery rate (FDR) was calculated by the method of Benjamini and Hochberg.

TABLE 7
Significant changes in miRNA plasma from patients with
moderate heart failure as compared to healthy controls

| | Normalized Read | | | | |
|---|---|---|---|---|---|
| | Frequency | | | | |
| miRNA | | Healthy | Fold | | |
| Cistron | Stable HF | Controls | Change | P value | FDR |
| mir-375(1) | 0.000067 | 0.000360 | −5.38 | 7.80E−05 | 1.70E−02 |
| mir-331(1) | 0.000256 | 0.000136 | 1.88 | 2.04E−04 | 1.70E−02 |
| mir-3613(1) | 0.000056 | 0.000140 | −2.49 | 2.23E−04 | 1.70E−02 |
| mir-210(1) | 0.000335 | 0.000179 | 1.88 | 3.09E−04 | 1.77E−02 |
| mir-141(2) | 0.000139 | 0.000293 | −2.10 | 4.39E−04 | 1.83E−02 |
| mir-769(1) | 0.000126 | 0.000261 | −2.07 | 5.87E−04 | 1.83E−02 |
| mir-181a-1(4) | 0.004309 | 0.007619 | −1.77 | 6.54E−04 | 1.83E−02 |
| mir-10a(1) | 0.000633 | 0.001296 | −2.05 | 7.04E−04 | 1.83E−02 |
| mir-203(1) | 0.000020 | 0.000090 | −4.53 | 7.18E−04 | 1.83E−02 |
| mir-1180(1) | 0.000012 | 0.000003 | 3.95 | 8.04E−04 | 1.84E−02 |
| mir-143(2) | 0.001592 | 0.003432 | −2.16 | 8.92E−04 | 1.86E−02 |
| mir-33a(1) | 0.000550 | 0.000282 | 1.95 | 1.11E−03 | 2.12E−02 |
| mir-30b(2) | 0.014103 | 0.020880 | −1.48 | 1.49E−03 | 2.63E−02 |
| mir-1908(1) | 0.000012 | 0.000006 | 2.10 | 1.66E−03 | 2.71E−02 |
| mir-151(1) | 0.009700 | 0.015415 | −1.59 | 2.68E−03 | 3.72E−02 |
| mir-624(1) | 0.000020 | 0.000012 | 1.69 | 2.73E−03 | 3.72E−02 |
| mir-132(2) | 0.000100 | 0.000174 | −1.75 | 2.76E−03 | 3.72E−02 |
| mir-584(1) | 0.001065 | 0.002044 | −1.92 | 3.59E−03 | 4.57E−02 |
| mir-296(1) | 0.000012 | 0.000006 | 1.81 | 4.04E−03 | 4.87E−02 |
| mir-181c(2) | 0.000363 | 0.000554 | −1.53 | 4.40E−03 | 5.03E−02 |
| mir-34b(2) | 0.000002 | 0.000007 | −4.34 | 5.11E−03 | 5.36E−02 |
| mir-205(1) | 0.000006 | 0.000022 | −3.37 | 5.15E−03 | 5.36E−02 |
| mir-196a-1(3) | 0.000039 | 0.000013 | 2.98 | 5.40E−03 | 5.38E−02 |
| mir-200a(3) | 0.000063 | 0.000130 | −2.08 | 1.04E−02 | 9.91E−02 |
| mir-324(1) | 0.000768 | 0.000554 | 1.39 | 1.14E−02 | 1.04E−01 |
| mir-3158(1) | 0.000010 | 0.000005 | 1.99 | 1.18E−02 | 1.04E−01 |
| mir-1914(1) | 0.000003 | 0.000001 | 2.08 | 1.32E−02 | 1.12E−01 |
| mir-597(1) | 0.000003 | 0.000001 | 2.53 | 1.39E−02 | 1.14E−01 |
| mir-744(1) | 0.002203 | 0.003207 | −1.46 | 1.46E−02 | 1.16E−01 |
| mir-144(2) | 0.160469 | 0.065035 | 2.47 | 1.92E−02 | 1.46E−01 |
| mir-1294(1) | 0.000010 | 0.000005 | 2.01 | 2.10E−02 | 1.55E−01 |
| mir-3679(1) | 0.000004 | 0.000002 | 2.21 | 2.21E−02 | 1.58E−01 |
| mir-339(1) | 0.001267 | 0.000858 | 1.48 | 2.30E−02 | 1.58E−01 |
| mir-378(1) | 0.000521 | 0.000886 | −1.70 | 2.35E−02 | 1.58E−01 |
| mir-192(4) | 0.000721 | 0.001305 | −1.81 | 2.90E−02 | 1.86E−01 |
| mir-582(1) | 0.000007 | 0.000017 | −2.28 | 2.92E−02 | 1.86E−01 |
| mir-340(1) | 0.001333 | 0.001779 | −1.33 | 3.90E−02 | 2.41E−01 |
| mir-641(1) | 0.000011 | 0.000019 | −1.70 | 4.06E−02 | 2.45E−01 |
| mir-133b(2) | 0.000011 | 0.000025 | −2.30 | 4.39E−02 | 2.58E−01 |
| mir-139(1) | 0.001203 | 0.000850 | 1.42 | 4.86E−02 | 2.76E−01 |
| mir-28(1) | 0.002467 | 0.003408 | −1.38 | 4.93E−02 | 2.76E−01 |
| mir-326(1) | 0.000428 | 0.000270 | 1.58 | 5.07E−02 | 2.77E−01 |
| mir-3940(1) | 0.000007 | 0.000005 | 1.51 | 5.33E−02 | 2.84E−01 |
| mir-140(1) | 0.003383 | 0.004413 | −1.30 | 5.56E−02 | 2.84E−01 |
| mir-10b(1) | 0.000564 | 0.001108 | −1.96 | 5.58E−02 | 2.84E−01 |
| mir-1537(1) | 0.000002 | 0.000003 | −1.75 | 5.98E−02 | 2.91E−01 |
| mir-424(2) | 0.000745 | 0.000532 | 1.40 | 5.99E−02 | 2.91E−01 |
| mir-26a-1(2) | 0.038479 | 0.051676 | −1.34 | 6.09E−02 | 2.91E−01 |
| mir-574(1) | 0.000534 | 0.000389 | 1.37 | 6.82E−02 | 3.16E−01 |
| mir-552(1) | 0.000002 | 0.000003 | −1.57 | 6.90E−02 | 3.16E−01 |
| mir-708(1) | 0.000002 | 0.000005 | −2.47 | 7.16E−02 | 3.22E−01 |
| mir-335(1) | 0.000953 | 0.001249 | −1.31 | 7.33E−02 | 3.23E−01 |
| mir-101-1(2) | 0.005644 | 0.008370 | −1.48 | 7.51E−02 | 3.23E−01 |
| mir-1306(1) | 0.000080 | 0.000060 | 1.33 | 7.67E−02 | 3.23E−01 |
| mir-3177(1) | 0.000012 | 0.000018 | −1.51 | 7.76E−02 | 3.23E−01 |
| mir-3138(1) | 0.000013 | 0.000009 | 1.49 | 8.01E−02 | 3.28E−01 |
| mir-3617(1) | 0.000007 | 0.000012 | −1.72 | 8.71E−02 | 3.44E−01 |
| mir-550-1(2) | 0.000055 | 0.000040 | 1.40 | 8.84E−02 | 3.44E−01 |
| mir-124-1(3) | 0.000010 | 0.000003 | 2.93 | 8.96E−02 | 3.44E−01 |
| mir-3127(1) | 0.000006 | 0.000004 | 1.41 | 9.01E−02 | 3.44E−01 |
| mir-128-1(2) | 0.000997 | 0.001310 | −1.31 | 9.28E−02 | 3.48E−01 |
| mir-186(1) | 0.005126 | 0.003935 | 1.30 | 1.04E−01 | 3.81E−01 |
| mir-3173(1) | 0.000005 | 0.000007 | −1.41 | 1.05E−01 | 3.81E−01 |
| mir-3174(1) | 0.000002 | 0.000001 | 1.68 | 1.12E−01 | 4.02E−01 |
| mir-589(1) | 0.000046 | 0.000062 | −1.33 | 1.17E−01 | 4.13E−01 |
| mir-3157(1) | 0.000004 | 0.000003 | 1.49 | 1.20E−01 | 4.15E−01 |
| mir-873(2) | 0.000007 | 0.000003 | 2.02 | 1.24E−01 | 4.24E−01 |
| mir-1287(1) | 0.000013 | 0.000018 | −1.36 | 1.29E−01 | 4.33E−01 |
| mir-874(1) | 0.000057 | 0.000042 | 1.34 | 1.33E−01 | 4.36E−01 |
| mir-345(1) | 0.000177 | 0.000143 | 1.24 | 1.33E−01 | 4.36E−01 |
| mir-26b(1) | 0.015799 | 0.019881 | −1.26 | 1.42E−01 | 4.56E−01 |
| mir-155(1) | 0.000312 | 0.000387 | −1.24 | 1.46E−01 | 4.56E−01 |
| mir-22(1) | 0.031886 | 0.026007 | 1.23 | 1.47E−01 | 4.56E−01 |
| mir-129-1(2) | 0.000003 | 0.000005 | −1.87 | 1.47E−01 | 4.56E−01 |
| mir-96(3) | 0.000454 | 0.000624 | −1.38 | 1.52E−01 | 4.59E−01 |
| mir-2116(1) | 0.000002 | 0.000003 | −1.72 | 1.52E−01 | 4.59E−01 |
| mir-511-1(2) | 0.000005 | 0.000008 | −1.58 | 1.61E−01 | 4.80E−01 |
| mir-30a(4) | 0.017029 | 0.020191 | −1.19 | 1.67E−01 | 4.85E−01 |
| mir-498(46) | 0.000006 | 0.000011 | −1.71 | 1.67E−01 | 4.85E−01 |
| mir-328(1) | 0.000726 | 0.000563 | 1.29 | 1.70E−01 | 4.85E−01 |
| mir-1284(1) | 0.000004 | 0.000005 | −1.46 | 1.75E−01 | 4.94E−01 |
| mir-1304(1) | 0.000030 | 0.000041 | −1.34 | 1.77E−01 | 4.95E−01 |
| mir-103-1(2) | 0.030347 | 0.023528 | 1.29 | 1.93E−01 | 5.21E−01 |
| mir-3909(1) | 0.000003 | 0.000002 | 1.50 | 1.93E−01 | 5.21E−01 |
| mir-3187(1) | 0.000007 | 0.000010 | −1.41 | 1.96E−01 | 5.21E−01 |
| mir-590(1) | 0.000196 | 0.000276 | −1.40 | 2.03E−01 | 5.21E−01 |
| mir-149(1) | 0.000002 | 0.000002 | 1.60 | 2.03E−01 | 5.21E−01 |
| mir-551b(1) | 0.000014 | 0.000019 | −1.39 | 2.05E−01 | 5.21E−01 |
| mir-134(41) | 0.005418 | 0.007474 | −1.38 | 2.05E−01 | 5.21E−01 |
| mir-651(1) | 0.000016 | 0.000022 | −1.43 | 2.11E−01 | 5.21E−01 |
| mir-616(1) | 0.000007 | 0.000005 | 1.24 | 2.13E−01 | 5.21E−01 |
| mir-505(1) | 0.000270 | 0.000202 | 1.33 | 2.13E−01 | 5.21E−01 |
| mir-642(1) | 0.000002 | 0.000003 | −1.70 | 2.14E−01 | 5.21E−01 |
| mir-1250(1) | 0.000009 | 0.000013 | −1.45 | 2.14E−01 | 5.21E−01 |
| mir-146b(1) | 0.001093 | 0.001327 | −1.21 | 2.19E−01 | 5.28E−01 |
| mir-3942(1) | 0.000002 | 0.000003 | −1.45 | 2.21E−01 | 5.28E−01 |
| mir-3124(1) | 0.000003 | 0.000004 | −1.49 | 2.25E−01 | 5.28E−01 |
| mir-2355(1) | 0.000121 | 0.000097 | 1.25 | 2.26E−01 | 5.28E−01 |
| mir-3065(1) | 0.000015 | 0.000018 | −1.24 | 2.33E−01 | 5.40E−01 |
| mir-484(1) | 0.004415 | 0.003804 | 1.16 | 2.41E−01 | 5.49E−01 |
| mir-338(1) | 0.000125 | 0.000103 | 1.22 | 2.42E−01 | 5.49E−01 |
| mir-1301(1) | 0.000331 | 0.000396 | −1.20 | 2.49E−01 | 5.59E−01 |
| mir-130b(2) | 0.000775 | 0.000664 | 1.17 | 2.53E−01 | 5.59E−01 |
| mir-3194(1) | 0.000004 | 0.000002 | 1.66 | 2.54E−01 | 5.59E−01 |
| mir-3198(1) | 0.000003 | 0.000002 | 1.45 | 2.64E−01 | 5.71E−01 |
| mir-4326(1) | 0.000010 | 0.000013 | −1.35 | 2.69E−01 | 5.71E−01 |
| mir-423(1) | 0.011054 | 0.009474 | 1.17 | 2.71E−01 | 5.71E−01 |
| mir-34a(1) | 0.000084 | 0.000060 | 1.41 | 2.71E−01 | 5.71E−01 |
| mir-148a(1) | 0.005416 | 0.006564 | −1.21 | 2.72E−01 | 5.71E−01 |
| mir-1256(1) | 0.000007 | 0.000009 | −1.28 | 2.81E−01 | 5.80E−01 |
| mir-760(1) | 0.000136 | 0.000093 | 1.47 | 2.84E−01 | 5.80E−01 |
| mir-190a(1) | 0.000087 | 0.000068 | 1.27 | 2.86E−01 | 5.80E−01 |
| mir-370(1) | 0.000350 | 0.000265 | 1.32 | 2.86E−01 | 5.80E−01 |
| mir-652(1) | 0.002809 | 0.002398 | 1.17 | 2.94E−01 | 5.91E−01 |
| mir-3120(1) | 0.000024 | 0.000019 | 1.21 | 3.06E−01 | 6.09E−01 |
| mir-3115(1) | 0.000004 | 0.000003 | 1.39 | 3.15E−01 | 6.21E−01 |
| mir-148b(1) | 0.006010 | 0.007042 | −1.17 | 3.17E−01 | 6.21E−01 |
| mir-1468(1) | 0.000008 | 0.000011 | −1.32 | 3.22E−01 | 6.24E−01 |
| mir-490(1) | 0.000006 | 0.000004 | 1.56 | 3.29E−01 | 6.26E−01 |
| mir-491(1) | 0.000046 | 0.000038 | 1.22 | 3.31E−01 | 6.26E−01 |
| mir-95(1) | 0.000007 | 0.000009 | −1.31 | 3.32E−01 | 6.26E−01 |
| mir-605(1) | 0.000005 | 0.000006 | −1.31 | 3.34E−01 | 6.26E−01 |
| mir-3140(1) | 0.000003 | 0.000002 | 1.30 | 3.39E−01 | 6.28E−01 |
| mir-3074(1) | 0.000013 | 0.000011 | 1.25 | 3.40E−01 | 6.28E−01 |
| mir-127(8) | 0.002142 | 0.002768 | −1.29 | 3.51E−01 | 6.41E−01 |
| mir-3143(1) | 0.000005 | 0.000004 | 1.14 | 3.53E−01 | 6.41E−01 |
| mir-185(1) | 0.006634 | 0.005778 | 1.15 | 3.69E−01 | 6.56E−01 |
| mir-1249(1) | 0.000008 | 0.000010 | −1.24 | 3.72E−01 | 6.56E−01 |
| mir-556(1) | 0.000044 | 0.000037 | 1.21 | 3.78E−01 | 6.56E−01 |
| mir-21(1) | 0.102972 | 0.117386 | −1.14 | 3.81E−01 | 6.56E−01 |
| mir-1270-1(1) | 0.000004 | 0.000003 | 1.22 | 3.81E−01 | 6.56E−01 |
| mir-1270-2(1) | 0.000004 | 0.000003 | 1.22 | 3.81E−01 | 6.56E−01 |
| mir-449a(3) | 0.000005 | 0.000003 | 1.40 | 3.81E−01 | 6.56E−01 |
| mir-576(1) | 0.000045 | 0.000053 | −1.17 | 3.84E−01 | 6.56E−01 |
| mir-580(1) | 0.000002 | 0.000003 | −1.44 | 3.89E−01 | 6.57E−01 |
| mir-885(1) | 0.000021 | 0.000015 | 1.41 | 3.90E−01 | 6.57E−01 |
| mir-107(1) | 0.002205 | 0.001851 | 1.19 | 3.93E−01 | 6.57E−01 |
| mir-942(1) | 0.000063 | 0.000055 | 1.14 | 3.97E−01 | 6.57E−01 |
| mir-29a(4) | 0.004754 | 0.005439 | −1.14 | 3.99E−01 | 6.57E−01 |
| mir-2110(1) | 0.000050 | 0.000043 | 1.17 | 4.06E−01 | 6.63E−01 |
| mir-146a(1) | 0.017872 | 0.019777 | −1.11 | 4.21E−01 | 6.84E−01 |
| mir-506(11) | 0.000003 | 0.000003 | −1.23 | 4.28E−01 | 6.87E−01 |
| mir-147(1) | 0.000003 | 0.000003 | −1.24 | 4.31E−01 | 6.87E−01 |
| mir-152(1) | 0.000920 | 0.001046 | −1.14 | 4.36E−01 | 6.87E−01 |
| mir-218-1(3) | 0.000005 | 0.000004 | 1.39 | 4.36E−01 | 6.87E−01 |
| mir-610(1) | 0.000002 | 0.000002 | 1.18 | 4.39E−01 | 6.87E−01 |
| mir-643(1) | 0.000004 | 0.000004 | −1.16 | 4.41E−01 | 6.87E−01 |
| mir-92b(1) | 0.000083 | 0.000099 | −1.20 | 4.49E−01 | 6.95E−01 |
| mir-98(13) | 0.104861 | 0.094681 | 1.11 | 4.57E−01 | 7.02E−01 |
| mir-1296(1) | 0.000012 | 0.000014 | −1.21 | 4.60E−01 | 7.02E−01 |
| mir-1277(1) | 0.000310 | 0.000368 | −1.19 | 4.80E−01 | 7.27E−01 |
| mir-208b(1) | 0.000002 | 0.000002 | −1.39 | 4.88E−01 | 7.35E−01 |
| mir-3176(1) | 0.000003 | 0.000002 | 1.39 | 5.00E−01 | 7.48E−01 |
| mir-3605(1) | 0.000008 | 0.000009 | −1.15 | 5.13E−01 | 7.49E−01 |
| mir-32(1) | 0.000435 | 0.000385 | 1.13 | 5.14E−01 | 7.49E−01 |
| mir-766(1) | 0.000285 | 0.000254 | 1.12 | 5.15E−01 | 7.49E−01 |
| mir-361(1) | 0.001228 | 0.001337 | −1.09 | 5.19E−01 | 7.49E−01 |
| mir-187(1) | 0.000002 | 0.000003 | −1.31 | 5.19E−01 | 7.49E−01 |
| mir-223(1) | 0.032189 | 0.028013 | 1.15 | 5.20E−01 | 7.49E−01 |
| mir-196b(1) | 0.000119 | 0.000109 | 1.10 | 5.23E−01 | 7.49E−01 |
| mir-1255a(1) | 0.000009 | 0.000008 | 1.19 | 5.38E−01 | 7.65E−01 |
| mir-188(8) | 0.000870 | 0.000789 | 1.10 | 5.41E−01 | 7.65E−01 |
| mir-3667(1) | 0.000002 | 0.000003 | −1.37 | 5.58E−01 | 7.78E−01 |
| mir-2278(1) | 0.000003 | 0.000003 | 1.12 | 5.61E−01 | 7.78E−01 |
| mir-7-1(3) | 0.000350 | 0.000314 | 1.12 | 5.63E−01 | 7.78E−01 |
| mir-197(1) | 0.000576 | 0.000529 | 1.09 | 5.64E−01 | 7.78E−01 |
| mir-629(1) | 0.000177 | 0.000205 | −1.16 | 5.69E−01 | 7.80E−01 |
| mir-221(2) | 0.016449 | 0.015106 | 1.09 | 5.87E−01 | 7.92E−01 |
| mir-195(2) | 0.000238 | 0.000212 | 1.13 | 5.88E−01 | 7.92E−01 |
| mir-150(1) | 0.001137 | 0.001342 | −1.18 | 5.88E−01 | 7.92E−01 |
| mir-153-1(2) | 0.000020 | 0.000017 | 1.16 | 5.95E−01 | 7.97E−01 |
| mir-33b(1) | 0.000020 | 0.000017 | 1.16 | 6.21E−01 | 8.22E−01 |
| mir-199b(1) | 0.003124 | 0.003500 | −1.12 | 6.21E−01 | 8.22E−01 |
| mir-628(1) | 0.000067 | 0.000074 | −1.10 | 6.41E−01 | 8.33E−01 |
| mir-450a-1(4) | 0.000119 | 0.000130 | −1.09 | 6.41E−01 | 8.33E−01 |
| mir-126(1) | 0.045010 | 0.041696 | 1.08 | 6.45E−01 | 8.33E−01 |
| mir-625(1) | 0.000534 | 0.000482 | 1.11 | 6.51E−01 | 8.33E−01 |
| mir-320(1) | 0.009268 | 0.008738 | 1.06 | 6.57E−01 | 8.33E−01 |
| mir-504(1) | 0.000004 | 0.000003 | 1.15 | 6.57E−01 | 8.33E−01 |
| mir-25(3) | 0.022596 | 0.023826 | −1.05 | 6.63E−01 | 8.33E−01 |
| mir-2276(1) | 0.000002 | 0.000002 | 1.06 | 6.66E−01 | 8.33E−01 |
| mir-122(1) | 0.004962 | 0.004276 | 1.16 | 6.67E−01 | 8.33E−01 |
| mir-3912(1) | 0.000005 | 0.000005 | 1.08 | 6.69E−01 | 8.33E−01 |
| mir-224(2) | 0.000898 | 0.000807 | 1.11 | 6.70E−01 | 8.33E−01 |
| mir-598(1) | 0.000092 | 0.000101 | −1.09 | 6.74E−01 | 8.33E−01 |
| mir-135a-1(3) | 0.014884 | 0.013887 | 1.07 | 6.77E−01 | 8.33E−01 |
| mir-330(1) | 0.000159 | 0.000147 | 1.08 | 6.81E−01 | 8.34E−01 |
| mir-1271(1) | 0.000028 | 0.000026 | 1.10 | 6.90E−01 | 8.40E−01 |
| mir-579(1) | 0.000006 | 0.000006 | 1.10 | 7.06E−01 | 8.53E−01 |
| mir-208a(1) | 0.000003 | 0.000002 | 1.17 | 7.11E−01 | 8.53E−01 |
| mir-3677(1) | 0.000002 | 0.000002 | 1.18 | 7.11E−01 | 8.53E−01 |
| mir-627(1) | 0.000038 | 0.000041 | −1.09 | 7.19E−01 | 8.58E−01 |
| mir-9-1(3) | 0.000038 | 0.000035 | 1.09 | 7.43E−01 | 8.78E−01 |
| mir-219-1(2) | 0.000003 | 0.000003 | −1.12 | 7.44E−01 | 8.78E−01 |
| mir-499(1) | 0.000016 | 0.000017 | −1.07 | 7.57E−01 | 8.84E−01 |
| mir-3611(1) | 0.000005 | 0.000006 | −1.09 | 7.64E−01 | 8.84E−01 |
| mir-216a(3) | 0.000002 | 0.000002 | 1.28 | 7.66E−01 | 8.84E−01 |
| mir-138-1(2) | 0.000004 | 0.000004 | −1.07 | 7.71E−01 | 8.84E−01 |
| mir-17(12) | 0.060368 | 0.062662 | −1.04 | 7.73E−01 | 8.84E−01 |
| mir-191(2) | 0.023945 | 0.022820 | 1.05 | 7.77E−01 | 8.84E−01 |
| mir-142(1) | 0.018409 | 0.017602 | 1.05 | 7.79E−01 | 8.84E−01 |
| mir-99b(3) | 0.004314 | 0.004522 | −1.05 | 7.80E−01 | 8.84E−01 |
| mir-193a(4) | 0.000146 | 0.000158 | −1.08 | 7.93E−01 | 8.91E−01 |
| mir-342(1) | 0.001016 | 0.001057 | −1.04 | 8.00E−01 | 8.91E−01 |
| mir-2277(1) | 0.000009 | 0.000008 | 1.10 | 8.03E−01 | 8.91E−01 |
| mir-570(1) | 0.000007 | 0.000006 | 1.12 | 8.03E−01 | 8.91E−01 |
| mir-455(1) | 0.000002 | 0.000002 | 1.19 | 8.06E−01 | 8.91E−01 |
| mir-374a(4) | 0.004303 | 0.004457 | −1.04 | 8.16E−01 | 8.95E−01 |
| mir-15a(4) | 0.028761 | 0.027546 | 1.04 | 8.17E−01 | 8.95E−01 |
| mir-199a-1(3) | 0.009587 | 0.010060 | −1.05 | 8.23E−01 | 8.98E−01 |
| mir-3928(1) | 0.000012 | 0.000012 | −1.02 | 8.60E−01 | 9.32E−01 |
| mir-23a(6) | 0.049882 | 0.048777 | 1.02 | 8.63E−01 | 9.32E−01 |
| mir-3150(1) | 0.000002 | 0.000002 | −1.08 | 8.74E−01 | 9.38E−01 |
| let-7i(1) | 0.017185 | 0.016820 | 1.02 | 8.76E−01 | 9.38E−01 |
| mir-204(1) | 0.000017 | 0.000016 | 1.03 | 8.92E−01 | 9.45E−01 |
| mir-3136(1) | 0.000002 | 0.000002 | 1.08 | 8.97E−01 | 9.45E−01 |
| mir-190b(1) | 0.000012 | 0.000012 | 1.00 | 9.01E−01 | 9.45E−01 |
| mir-483(1) | 0.000027 | 0.000026 | 1.05 | 9.03E−01 | 9.45E−01 |
| mir-301a(2) | 0.000969 | 0.000948 | 1.02 | 9.04E−01 | 9.45E−01 |
| mir-3155(1) | 0.000002 | 0.000002 | 1.09 | 9.15E−01 | 9.52E−01 |
| mir-671(1) | 0.000116 | 0.000118 | −1.01 | 9.18E−01 | 9.52E−01 |
| mir-3688(1) | 0.000004 | 0.000004 | 1.00 | 9.26E−01 | 9.52E−01 |
| mir-130a(1) | 0.003539 | 0.003486 | 1.02 | 9.28E−01 | 9.52E−01 |
| mir-1-1(4) | 0.000613 | 0.000627 | −1.02 | 9.42E−01 | 9.60E−01 |
| mir-3200(1) | 0.000004 | 0.000004 | −1.00 | 9.44E−01 | 9.60E−01 |
| mir-1307(1) | 0.000952 | 0.000945 | 1.01 | 9.68E−01 | 9.81E−01 |
| mir-486(1) | 0.021700 | 0.021446 | 1.01 | 9.73E−01 | 9.81E−01 |
| mir-1179(1) | 0.000009 | 0.000009 | −1.01 | 9.94E−01 | 9.98E−01 |
| mir-3130(1) | 0.000003 | 0.000003 | −1.01 | 1.00E+00 | 1.00E+00 |

Having the largest increase in the circulation of advanced HF patients and being tissue-specific, myomirs have a distinctive advantage over the other, less elevated miRNAs for diagnostic purposes. Thus, we compared their levels to those of cardiac troponin I (cTnI) and B-type natriuretic peptide (BNP) protein levels, established biomarkers for myocardial injury and dysfunction, respectively. Higher levels of the heart-specific myomirs mir-208a(1), mir-208b(1) and mir-499(1) were positively correlated with cTnI (R=0.75, p=4.73*10⁻⁶; R=0.76, p=4.59*10⁻⁷; and R=0.6, p=8.86*10⁻⁵, respectively) but not correlated with BNP. The cTnI concentrations in serum of NF were below the detection limit of 0.01 ng/ml, except for one sample reaching 0.03 ng/ml, closely followed by a median of 0.04 ng/ml in 3M or 6M LVAD (IQR=0.05), a median of 0.09 ng/ml (IQR=0.12) in stable HF, a median of 0.5 ng/ml (IQR=1.18) in patients with advanced HF at LVAD implantation, and maximum concentrations with a median of 9.8 ng/ml (IQR=15.8) at LVAD explantation. In the supervised classification area under the ROC curve the heart-specific cistrons performed similar to cTnI. Together, these results support a role for circulating miRNAs as biomarkers of myocardial injury.

We used a small RNAseq protocol developed for parallel processing of large sample collections with limited amounts of input RNA to record the miRNA composition in heart tissue and in circulation in a large cohort of heart failure (HF) patients and normal controls. Williams Z et al. (2013) Proc Natl Acad Sci USA 110:4255-4260; Hafner M et al. (2012) Methods 58:164-170; Farazi T A et al. (2012) Methods 58:171-187. Using the same method for myocardium and circulating miRNA profiling eliminated biases. Hafner M et al. (2011) RNA 17:1697-1712. otherwise affecting comparison of our data to other studies, which previously profiled either tissue or circulating miRNAs in HF, but never both.

In order to identify changes in myocardial miRNAs abundant enough to trigger measurable differences in mRNA expression by miRNA-mediated degradation, we at first considered miRNAs contributing to the top 85% sequence reads. For these highly expressed miRNAs the overall abundance in failing compared to normal postnatal myocardium differed not more than 2-fold, in agreement with a recent RNAseq tissue study by Yang et al. (2014) Circulation 129:1009-1021. The same miRNAs were also altered in heart development but changed up to 6-fold. These alterations in miRNA abundance resulted in an average of 1.02- to 1.20-fold miRNA-seed-dependent mRNA destabilization similar to observed values in mechanistic studies Grimson A et al. (2007) Mol Cell 27:91-105; Fang Z et al. (2011) PLoS ONE 6:e18067.

The composition of circulating small RNAs was dominated by miRNAs that are abundant in hematopoietic cells (Williams Z et al. (2013) Proc Natl Acad Sci US A 110:4255-4260) and/or the endothelium. The contribution of myomirs to all circulating miRNAs was less than 0.1% in healthy controls, patients with moderate and stable HF. However, the myomirs increased to over 1% in patients with advanced HF, and was reduced to nearly normal levels at 3 and 6 months after LVAD implantation. The myomirs are subdivided into the cardiac-specific mir-208a(1), mir-208b(1), and mir-499(1) and the broadly muscle-specific mir-1-1(4) and mir-133b(2), which are responsible for the circulating myomir background levels in healthy individuals. Hence, mir-1-1(4) and mir-133b(2), which together contributed 30% of all myocardial miRNAs, increased less than the cardiac-specific myomirs. However, the relative abundance of heart-specific myomirs in circulation followed closely the ratio determined in heart tissue.

Increased circulation of myomirs strongly correlated with increased cardiac troponin I (cTnI), but not BNP protein levels; these proteins are established diagnostic heart injury and heart function markers, respectively. Increases in circulating miRNAs upon cell damage have been detected by RT/PCR-based approaches for liver, brain and skeletal muscle. Laterza O F et al. (2009) Clin Chem 55:1977-1983) as well as the heart Corsten M F et al. B (2010) Circ Cardiovasc Genet 3:499-506; Ji X et al. N (2009) Clin Chem 55:1944-1949. In some instances they even performed better than established protein biomarkers Laterza O F et al. (2009) Clin Chem 55:1977-1983. Our analysis indicated that heart-specific myomirs performed similar to the highly sensitive cTnI assay.

Materials and Methods:

(i) Tissue Procurement: Human myocardial tissue samples were obtained from the National Human Tissue Resource Center (Philadelphia, Pa., USA), from Columbia University Medical Center, and after elective termination of pregnancy for non-medical reasons. Serum and plasma samples were obtained from Columbia University Medical Center; (ii) RNA Isolation: Total RNA from solid tissue and liquid samples was isolated with a modified TRIzol protocol and recovered by alcohol precipitation. Liquid sample RNA recovery included addition of glycogen for co-precipitation. Tissue total RNA was further purified by Qiagen RNeasy columns for bead array studies: (iii) Small RNA Sequencing and Gene Expression Analysis—The cDNA library preparation and annotation were done as described (Hafner M et al. (2011 RNA 17:1697-1712; Brown M et al. (2013) Front Genet 4:145; Farazi T A et al. (2012) Methods 58:171-187) with modifications for library preparations of serum and plasma samples. mRNA expression was assessed on Illumina HumanHT-12v4 bead arrays according to the manufacturer's instructions: (iv) The data was analyzed in the R statistical language. The functional studies testing miRNA regulation followed the approach by Grimson et al. Grimson A et al. (2007) Mol Cell 27:91-105. Differences in RNA quantification for unpaired samples were tested using the Kruskal-Wallis rank sum test and for paired samples using the Wilcoxon signed rank test. The differences in the cumulative distributions were tested using the one-sided Kolmogorov-Smirnov test: (v) SI Materials and Methods: (a) Tissue Procurement. Nonfailing (NF) postnatal cardiac tissue was obtained from the National Human Tissue Resource Center (National Disease Research Interchange, Philadelphia). Five fetal heart specimens (gestational age 19-24 wk) were obtained after elective termination of pregnancy for nonmedical reasons. Failing myocardial samples and blood for serum and EDTA-plasma preparation from patients with heart failure (HF) were obtained from the Columbia University Medical Center. Serum and EDTA-plasma samples of healthy controls were also obtained from the Columbia University Medical Center. The tissue samples were immediately flash frozen in liquid nitrogen upon harvesting and stored at −80° C. until processing: (b) RNA Isolation. Total RNA from tissue and plasma samples was isolated with a modified TRIzol protocol and recovered by ethanol precipitation. Tissue samples were homogenized in 20×volume of TRIzol using a mechanical bead mill. After thawing, the plasma samples were centrifuged at 16,000×g at 4° C. for 5 min to remove residual debris, and 500 μL, were homogenized by vortexing with 3×volume of TRIzol LS. After the initial homogenization and isopropanol precipitation, myocardial tissue samples were additionally treated with DNase I [0.2 U/μL final concentration (f.c.)] for 30 min at 37° C., and both myocardial and plasma samples were digested with proteinase K (100 μg/mL f.c. in a buffer containing 0.5% SDS) for 20 min at 42° C. before a second phenol chloroform extraction. The samples were precipitated twice in the presence of 0.3 M NaOAc (pH 5.2) with 3 volumes of 100% ethanol at −20° C. for at least 1 h, collected by centrifugation for 30 min at 16,000×g, and resuspended in RNase-free water. All precipitation steps of the plasma samples were done in the presence of glycogen at a final concentration of 40 μg/mL as a carrier. The RNA composition may vary according to the used RNA isolation protocol, and RNA isolations using the TRIzol protocol as described by the manufacturer without carrier skews the microRNA (miRNA) distribution in low concentration RNA samples. Kim Y K et al. (2012) Mol Cell 46(6):893-895; Hafner M, et al. (2012) Methods 58(2):164-170. However, using carrier glycogen, we did not observe any depletion of possibly affected miRNAs, e.g., miR-21. For the microarray studies, the RNA was additionally processed using Qiagen RNeasy columns as described in the manufacturer's manual.

The RNA concentration and purity was determined by microvolume UV spectrophotometry (NanoDrop; Thermo Scientific) or using the fluorometric Qubit RNA Assay (Molecular Probes; Life Technologies). The RNA integrity of the tissueRNAsamples was determined by a microchip based capillary electrophoresis (Agilent Bioanalyzer 2100): (c) sRNA Library Preparation and Analysis. The cDNA library preparation for the tissue samples was done according to our published protocol. Hafner M et al. (2012) Methods 58(2):164-170. Briefly, total RNA was ligated to a 3′-oligonucleotide adapter containing a 5-nt barcode at the 5′-end allowing the pooling of up to 20 samples in one flow lane and at the same time preserving strand orientation and minimizing intersample variation. An equimolar mixture of 10 synthetic 22-nt calibrator oligoribonucleotides were spiked in at this step. Calibrators are synthetic oligoribonucleotides spiked-in into samples (for sequences and details, Hafner M et al. (2012) Methods 58(2):164-170. Note: No oligoribonucleotide cocktail was spiked-in into library 8 (serum and plasma library). These spike-in controls have no match in the human genome and served as quality control and quantification. The samples were pooled and size-selected by 15% denaturing polyacrylamide gel electrophoresis and gel eluted, followed by 5′-adapter ligation and another gel purification. The ligated RNA was reverse transcribed using SuperScript III reverse transcriptase (Life Technologies) and the RNA was hydrolyzed by alkaline hydrolysis. For the tissue libraries, the RNA input was 1-2 μg and the amount of spiked-in oligoribonucleotide mixture 0.25 fmol each per microgram of total RNA. The input for the serum or plasma samples was the total RNA from 0.5 mL starting material, and the oligoribonucleotide amount was reduced to 0.005 fmol for each calibrator per sample. One sRNA cDNA library for plasma and serum samples (library 8) was not spiked with calibrator oligonucleotides.

In addition, the tissue libraries were also spiked-in with radiolabeled size markers that facilitated size selection (19 and 24 nt). These were digested with PmeI after PCR amplification; the serum and plasma samples did not contain size markers. The libraries were amplified by 7-12 cycles (tissue) or 12-16 cycles (plasma) of PCR, and loaded onto a 2.5% (wt/vol) agarose gel for gel purification using the Qiagen Gel extraction kit. The eluted cDNA was sequenced on an Illumina GAIIx or HiSeq 2000 sequencer in the Genomic Core Facility at The Rockefeller University. Bioinformatics Analysis of RNA Sequencing. The FASTQ output files from the HiSeq 2000 were analyzed using a pipeline as described previously. Farazi T A, et al. (2012) Methods 58(2):171-187; Brown M et al. (2013) Front Genet 4:145. The files were demultiplexed, the 3′-adapters trimmed, and sequences between 16 and 35 nt aligned to the human genome build 37 allowing one mismatch, and allowing two mismatches to curated RNA transcriptomes for miRNAs as well as rRNAs, tRNAs, small cytoplasmic RNAs (scRNAs), small Cajal body-specific RNAs (scaRNAs), snRNAs, small nucleolar RNAs (snoRNAs), circular RNAs (circRNAs), and bacterial plasmid references used in recombinant protein expression. Farazi T A, et al. (2012) Methods 58(2):171-187; Brown M et al. (2013) Front Genet 4:145. The reads were aligned with the short read aligner Burrows-Wheeler Alignment tool. Li H et al. (2009) Bioinformatics 25(14):1754-1760.

For the unsupervised clustering analysis, we restricted the set of miRNAs to the ones within the top 85% sequence reads in at least one sample, for which we can measure regulatory effects. The dataset included 10 technical replicates that clustered reproducibly. Unsupervised hierarchical clustering was performed using Euclidean distance and complete linkage for columns (samples) and rows (miRNAs or mRNAs) unless indicated otherwise; for the sake of clarity the row dendrograms were removed from the figures (with exception of some of the figures—Unsupervised hierarchical clustering of external RNA standards. Ten synthetic 22-nt external reference oligoribonucleotides (calibrators) were added in equimolar amounts to the sample RNA during the sRNA cDNA preparation. These calibrators can be used for miRNA quantification and library quality control. The calibrators were designed to reflect the different ligation efficiencies of naturally occurring (small) RNAs, with calibrators like Cal05 or Cal08 being less efficiently carried through the library preparation than others. The calibrator reads for all 14 libraries that were supplemented with external reference RNA were converted to the log 2 read frequencies and subjected to agglomerative hierarchical clustering using Euclidean distance metrics and the complete linkage algorithm for column and row clustering. Please note that library 8 (serum and plasma samples) was not spiked-in with external standards and as such is not shown here.)

The differential expression (or levels in the case of plasma samples) analysis was done with the R/Bioconductor package edgeR (Version 3.3.5). Robinson M D et al. (2010) Bioinformatics 26(1):139-140; Robinson M D et al. (2007) Bioinformatics 23(21):2881-2887; Robinson M D et al. (2008) Biostatistics 9(2):321-332. The reads were normalized using the weighted trimmed mean of M values (Robinson M D et al. (2010) Genome Biol 11(3):R25) and normalized for library size. We kept only miRNAs with one read per million reads in at least five samples for the differential expression/levels analysis. The differences were tested using the exact test for unpaired samples, or by an additive generalized linear model (GLM) for paired samples with the patients as the blocking factor. The read variation was estimated using tagwise or common dispersion for the exact test and the GLM, respectively. In the biological myocardial replicates this variation was typical for what has been reported in other RNA sequencing (RNAseq) studies (the biological coefficient of variation was between 0.44 and 0.51) (McCarthy D J et al. (2012) Nucleic Acids Res 40(10):4288-4297) and the variability in the plasma samples was higher (the biological coefficient of variation ranged from 0.59 to 0.84). Differences were considered significant below a false discovery rate (FDR) (Benjamini Y, et al. (1995). Journal of the Royal Statistical Society Series B (Methodological) 57(1):289-300) of 10%. Bioinformatics Analysis of mRNA Expression Arrays. The mRNA gene expression experiments of selected subsamples were performed on the HumanHT-12v4 bead arrays from Illumina. For the in vitro transcription and RNA labeling, 200 μg total RNA were used as input with the Ambion MessageAmp Premier RNA Amplification Kit (Life Technologies), and the amplified RNA (aRNA) quality checked by microfluidic analysis (Bioanalyzer 2100). For each sample, 750 ng aRNA was hybridized to a section of the Illumina BeadArrays. aRNA synthesis and hybridization were done by the Genomics Core Facility at The Rockefeller University. The arrays were scanned on a BeadScan station, and the analysis was based on the bead level data using R (Version 3.1) (R Core Team (2013) R: A language and environment for statistical computing (R Foundation for Statistical Computing, Vienna, Austria). Available at www.R-project.org. Accessed Jul. 1, 2014) and the Bioconductor 2.13 beadarray (2.12.0) (Dunning M J et al. (2007) Bioinformatics 23(16):2183-2184; Dunning M J et al. (2008) BMC bioinformatics 9:85; Cairns J M et al. (2008) Bioinformatics 24:2921-2922; Barbosa-Morais N L et al. (2010) Nucleic Acids Res 38:e17), lumi (2.14.1) (Du P, Kibbe W A et al. (2008) Bioinformatics 24(13):1547-1548; Lin S M et al. (2008) Nucleic Acids Res 36(2):e11), and limma (3.18.3) (Smyth G K (2004) Stat Appl Genet Mol Biol 3(1):Article3; Smyth G K (2005) Bioinformatics and Computational Biology Solutions using R and Bioconductor, eds Gentleman R, Carey V, Dudoit S, Irizarry R, Huber W (Springer, New York), pp 397-420; Ritchie M E et al. (2006) BMC Bioinformatics 7:261) packages. The arrays were transformed by variance-stabilizing transformation (Lin S M et al. (2008) Nucleic Acids Res 36(2):e11) followed by robust spline normalization probes with a match category “bad” or “no match” to the genome or transcriptome were removed after normalization (Ritchie M E et al. (2011) PLOS Comput Biol 7(12):e1002276), as were probes matching to the Y chromosome due to the uneven or unknown sex distribution. The moderated t statistic was used to test for differential expression (Smyth G K (2004) Stat Appl Genet Mol Biol 3(1):Article3). Reported expression differences are for an FDR of 10% [Benjamini and Hochberg (Benjamini Y et al. (1995) Journal of the Royal Statistical Society Series B (Methodological) 57(1):289-300.) unless stated otherwise. Analysis of miRNA-mRNA Correlations. The functional studies testing miRNA regulation followed the approach by Grimson et al. (Grimson A et al. (2007) Mol Cell 27(1):91-105). We only considered probes with intensity at least above the median, allowed only one miRNA target site per miRNA and transcript, and did not allow nested sites. We also tested the effects on highly expressed genes, defined as probe intensities above the 75th percentile. The 3′UTRs and the coding sequences were downloaded from Ensembl (Versions 67 and 71, respectively), and in cases of multiple transcripts per gene the longest isoform was used. Cardiac Troponin I and B-Type Natriuretic Peptide ELISAs. Cardiac troponin I (cTnI) and B-type natriuretic peptide (BNP) were both measured by a chemiluminescent microparticle immunoassay performed for quantitative determination of BNP in plasma or cTnI in serum using the ARCHITECT iSystem (Abbott). Other Statistical Analyses. All statistical analyses were done in the R statistical language. Differences in RNA quantification for unpaired samples were tested using the Kruskal-Wallis rank sum test; for paired samples, the Wilcoxon signed rank test was used. The differences in the empirical cumulative distributions were tested using one-sided Kolmogorov-Smirnov. For all tests, an alpha level of 0.05 was considered significant. To compare the performance of circulating miRNAs and cTnI as biomarker, a two-class area under the curve was computed.

### Accession Numbers

The sequencing and gene expression data were deposited in the NCBI Gene Expression Omnibus (GEO) under accession GSE53081.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

1. A method for identifying a subject in need of treatment for a cardiovascular disease, the method comprising the steps of:
(a) obtaining a sample from the subject;
(b) assaying the levels of a plurality of miRNAs in the sample, wherein the plurality of miRNAs comprise 3 or more miRNAs listed in Table 1 (SEQ ID NOs: 1-504), or in any of Tables 3-7;
(c) comparing the levels obtained in step (b) with the levels of the plurality of miRNAs in a control sample; and
(d) treating the subject for a cardiovascular disease, if the levels of at least 2 miRNAs obtained in step (b) are at least 2 fold of their levels in the control sample.

2. The method of claim 1, wherein the at least 2 miRNAs in step (d) are any combination of two or more miRNAs selected from the group consisting of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-203 and miR-126.

3. The method of claim 2, wherein the miRNAs are selected from the group consisting of miR-208a, miR-208b, miR-499 or mixtures thereof.

4. The method of claim 1, wherein the at least 2 miRNAs in step (d) are any combination of two or more miRNAs selected from the group consisting of miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320.

5. The method of claim 1, wherein in step (d) the subject is treated for a cardiovascular disease, if the levels of at least 2 miRNAs obtained in step (b) are at least 10 fold of their levels in the control sample.

6. The method of claim 1, wherein in step (d) the subject is treated for a cardiovascular disease, if the levels of at least 2 miRNAs obtained in step (b) are between about 10 fold and about 200 fold of their levels in the control sample.

7. The method of claim 1, wherein the sample is a plasma or serum sample.

8. The method of claim 1, wherein the cardiovascular disease is heart failure.

9. The method of claim 8, wherein the heart failure is advanced or stable heart failure.

10. The method of claim 1, wherein the subject is treated with a pharmacologic composition, a medical device, surgery, or any combination thereof.

11. The method of claim 10, wherein the medical device is a left ventricular assist device (LVAD).

12. The method of claim 11, wherein the subject is treated with the LVAD for at least three months.

13. The method of claim 1, wherein the subject is treated with antisense oligonucleotides targeting at least one miRNA selected from the group consisting of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-203 and miR-126.

14. The method of claim 13, wherein the miRNA is selected from the group consisting of miR-208a, miR-208b, miR-499 or mixtures thereof.

15. The method of claim 1, wherein the subject is treated with antisense oligonucleotides targeting at least one miRNA selected from the group consisting of miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320.

16. The method of claim 1, wherein the levels of the plurality of microRNA are determined by RNA sequencing, microarray profiling or real-time PCR.

17. The method of claim 1, wherein the control sample is from a healthy subject or a plurality of healthy subjects.

18. A method for assessing efficacy of a therapy for a cardiovascular disease in a patient, the method comprising the steps of:
(a) obtaining a first sample from the patient before initiation of the therapy;
(b) assaying the levels of a plurality of miRNAs in the first sample, wherein the plurality of miRNAs comprise 3 or more miRNAs listed in Table 1 (SEQ ID NOs: 1-504), or in any of Tables 3-7;
(c) obtaining a second sample from the patient after initiation of the therapy;
(d) assaying the levels of the plurality of miRNAs in the second sample; and
(e) comparing the levels of step (b) with the levels of step (d).

19. The method of claim 18, wherein the therapy is effective, if the levels of at least 2 miRNAs obtained in step (d) are less than about 20% of their levels obtained in step (b).

20. The method of claim 18, wherein the at least 2 miRNAs in step (f) are any combination of two or more miRNAs selected from the group consisting of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-203 and miR-126.

21. The method of claim 20, wherein the miRNAs are selected from the group consisting of miR-208a, miR-208b, miR-499 or mixtures thereof.

22. The method of claim 18, wherein the at least 2 miRNAs in step (f) are any combination of two or more miRNAs selected from the group consisting of miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320.

23. The method of claim 18, wherein the therapy is continued if the levels of at least 2 miRNAs obtained in step (d) are less than about 10% of their levels obtained in step (b).

24. The method of claim 18, wherein the sample is a plasma or serum sample.

25. The method of claim 18, wherein the cardiovascular disease is heart failure.

26. The method of claim 18, wherein the therapy is pharmacologic intervention, implantation of a medical device, surgery, or any combination thereof.

27. The method of claim 26, wherein the medical device is a left ventricular assist device (LVAD).

28. The method of claim 27, wherein the therapy with the LVAD is carried out for at least three months.

29. The method of claim 18, wherein the therapy is treatment with antisense oligonucleotides targeting at least one miRNA selected from the group consisting of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-203 and miR-126.

30. The method of claim 29, wherein the miRNAs are selected from the group consisting of miR-208a, miR-208b, miR-499 or mixtures thereof.

31. The method of claim 18, wherein the therapy is treatment with antisense oligonucleotides targeting at least one miRNA selected from the group consisting of miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320.

32. The method of claim 18, wherein the levels of the plurality of microRNA are determined by RNA sequencing, microarray profiling or real-time PCR.

33. A method for assessing efficacy of a therapy for a cardiovascular disease in a patient, the method comprising the steps of:
(a) obtaining a first sample from the patient before initiation of the therapy;
(b) assaying the levels of a plurality of miRNAs in the first sample, wherein the plurality of miRNAs comprises 3 or more miRNAs selected from the group consisting of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126, miR-203, miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320;
(c) obtaining a second sample from the patient after initiation of the therapy;
(d) testing the second sample for levels of the plurality of microRNAs; and
(e) comparing the levels of step (b) with the levels of step (d).

34. The method of claim 33, wherein the plurality of miRNAs comprises two or more myomirs.

35. The method of claim 33, wherein the miRNAs are selected from the group consisting of miR-208a, miR-208b, miR-499 or mixtures thereof.

36. The method of claim 33, wherein the sample is a plasma or serum sample.

37. The method of claim 33, wherein the cardiovascular disease is heart failure.

38. The method of claim 33, wherein the therapy is implantation of an LVAD.

39. The method of claim 38, wherein the therapy with the LVAD is carried out for at least three months.

40. The method of claim 33, wherein the levels of the plurality of microRNA are determined by RNA sequencing, microarray profiling or real-time PCR.

41. A method for evaluating a cardiovascular disease or monitoring progression of a cardiovascular disease in a patient, the method comprising the steps of:
(a) obtaining a sample from the patient;
(b) assaying the levels of a plurality of miRNAs in the sample, wherein the plurality of miRNAs comprises 3 or more miRNAs selected from the group consisting of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126, miR-203, miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320; and
(c) comparing the levels of step (b) with the levels of the plurality of miRNAs in a control sample.

42. A method for evaluating a cardiovascular disease or monitoring progression of a cardiovascular disease in a patient, the method comprising the steps of:
(a) obtaining a sample from the patient;
(b) testing the sample for levels of a plurality of miRNAs, wherein the plurality of miRNAs comprises 3 or more miRNAs listed in Table 1 (SEQ ID NOs: 1-504), or in any of Tables 3-7; and
(c) comparing the levels of step (b) with the levels of the plurality of miRNAs in a control sample.

43. The method of claim 41 or 42, wherein the plurality of miRNAs comprises two or more myomirs.

44. The method of claim 41 or 42, wherein the control sample is from a healthy subject or a plurality of healthy subjects.

45. The method of claim 41 or 42, wherein the sample is a plasma or serum sample.

46. The method of claim 41 or 42, wherein the cardiovascular disease is heart failure.

47. The method of claim 41 or 42, wherein the therapy is implantation of an LVAD.

48. The method of claim 47, wherein the therapy with the LVAD is carried out for at least three months.

49. The method of claim 41 or 42, wherein the levels of the plurality of microRNA are determined by RNA sequencing.

50. A kit comprising:
miRNA-specific primers for reverse transcribing or amplifying 3 or more miRNAs selected from Table 1, or selected from any of Tables 3-7, in a plasma or serum sample from a patient receiving treatment for a cardiovascular disease; and
instructions for measuring the 3 or more miRNAs for evaluating or monitoring the efficacy of a therapeutic intervention for treating a cardiovascular disease in the patient.

51. The kit of claim 50, wherein the kit comprises miRNA-specific primers for 3 or more miRNAs selected from miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126 and miR-203.

52. The method of claim 50, wherein the miRNAs are selected from the group consisting of miR-208a, miR-208b, miR-499 or mixtures thereof.

53. The kit of claim 50, wherein the kit comprises miRNA-specific primers for 3 or more miRNAs selected from miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320.

54. The kit of claim 50, further comprising a labeled-nucleic acid probe specific for each miRNA of the kit.
